(19) United States
(12) Patent Application Publication
(10) Pub. No.: US 2026/0115139 A1
(43) Pub. Date: Apr. 30, 2026

(54) Amino Acid-Containing Ionizable Lipids for the Delivery of Therapeutic Agents

(71) Applicant: NanoVation Therapeutics Inc., Vancouver (CA)

(72) Inventors: N.D. Prasad Atmuri, Vancouver (CA); Fariba Saadati, Vancouver (CA); Marco A. Clufolini, Vancouver (CA)

(73) Assignee: NanoVation Therapeutics Inc., Vancouver, BC (CA)

(21) Appl. No.: 19/115,832

(22) PCT Filed: Sep. 27, 2023
(86) PCT No.: PCT/CA2023/051274

§ 371 Date: Mar. 27, 2025

Related U.S. Application Data
(60) Provisional application No. 63/410,273, filed on Sep. 27, 2022.

(51) Int. Cl. A61K 9/1272; A61K 9/51; A61K 31/7088; C07C 233/47; C07C 235/12; C07C 323/12; C07C 323/25; C07C 323/60; C07D 207/16; C07D 317/24; C07D 317/28; C07D 403/12

(52) U.S. Cl. CPC A61K 9/1272; A61K 9/5123; A61K 31/7088; C07C 233/47; C07C 235/12; C07C 323/12; C07C 323/25; C07C 323/60; C07D 207/16; C07D 317/24; C07D 317/28; C07D 403/12

(57) ABSTRACT

Provided are lipids and nanoparticles containing such lipids and a cargo molecule, such as nucleic acid, methods to formulate said lipids with nucleic acids to produce lipid nanoparticles and chemical routes for making the lipids. The lipids may have the structure of Formula A as defined herein.

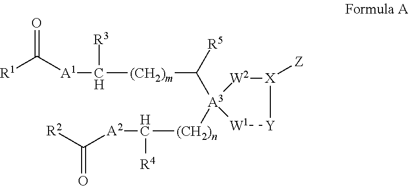

### TECHNICAL FIELD

Provided herein are lipids that may be formulated in a delivery vehicle so as to facilitate the encapsulation of a wide range of cargo therein, such as, without limitation, nucleic acids (e.g., RNA or DNA), proteins, peptides, pharmaceutical drugs and salts thereof.

### BACKGROUND

Nucleic acid-based therapeutics have enormous potential in medicine. To realize this potential, however, the nucleic acid must be delivered to a target site in a patient. This presents challenges since nucleic acid is rapidly degraded by enzymes in the plasma upon administration. Even if the nucleic acid is delivered to a disease site, there still remains the challenge of intracellular delivery. To address these problems, lipid nanoparticles have been developed that protect nucleic acid from such degradation and facilitate delivery across cellular membranes to gain access to the intracellular compartment, where the relevant translation machinery resides.

A key component of a lipid nanoparticle (LNP) is an ionizable lipid. The ionizable lipid is typically positively charged at low pH, which facilitates association with the negatively charged nucleic acid. However, the ionizable lipid is neutral at physiological pH, making it more biocompatible in biological systems. Further, it has been suggested that after the LNPs are taken up by a cell by endocytosis, the ionizability of these lipids at low pH enables endosomal escape. This in turn enables the nucleic acid to be released into the intracellular compartment.

An earlier example of a lipid nanoparticle product approved for clinical use and reliant on ionizable lipid is Onpattro®. Onpattro® is a lipid nanoparticle-based short interfering RNA (siRNA) drug for the treatment of polyneuropathies induced by hereditary transthyretin amyloidosis. Onpattro® is reliant on an ionizable lipid referred to as “DLin-MC3-DMA” or more commonly “MC3”, 1 (FIG. 1), by investigators. Furthermore, MC3 represents an evolution of a structurally related ionizable lipid, referred to by investigators as “KC2”, 2 (FIG. 1). MC3 is considered a state-of-the art ionizable lipid for the delivery of siRNA, requiring about 3 times less siRNA than KC2, although KC2 is superior in other applications, and it remains a valuable research tool.

While the foregoing ionizable lipids are especially efficacious for the delivery of siRNA-containing LNPs to hepatic cells, they are much less effective for the hepatic delivery of mRNA-containing LNPs. To illustrate, mRNA vaccines, including the COVID-19 Pfizer/BioNTech and Moderna vaccines, rely on lipid nanoparticles to deliver mRNA to the cytoplasm of liver cells. After entry into the host cell, the mRNA is transcribed to produce antigenic proteins. In the case of the COVID-19 vaccines, the mRNA encodes the highly immunogenic Sars-Cov-2 spike protein. Such vaccines, however, incorporate other types of ionizable lipids besides MC3 or KC2. In particular, the Pfizer/BioNTech vaccine comprises an ionizable lipid referred to as “ALC-0315”, 3 (Scheme 1), and the Moderna vaccine comprises an ionizable lipid referred to as “SM-102”, 4.

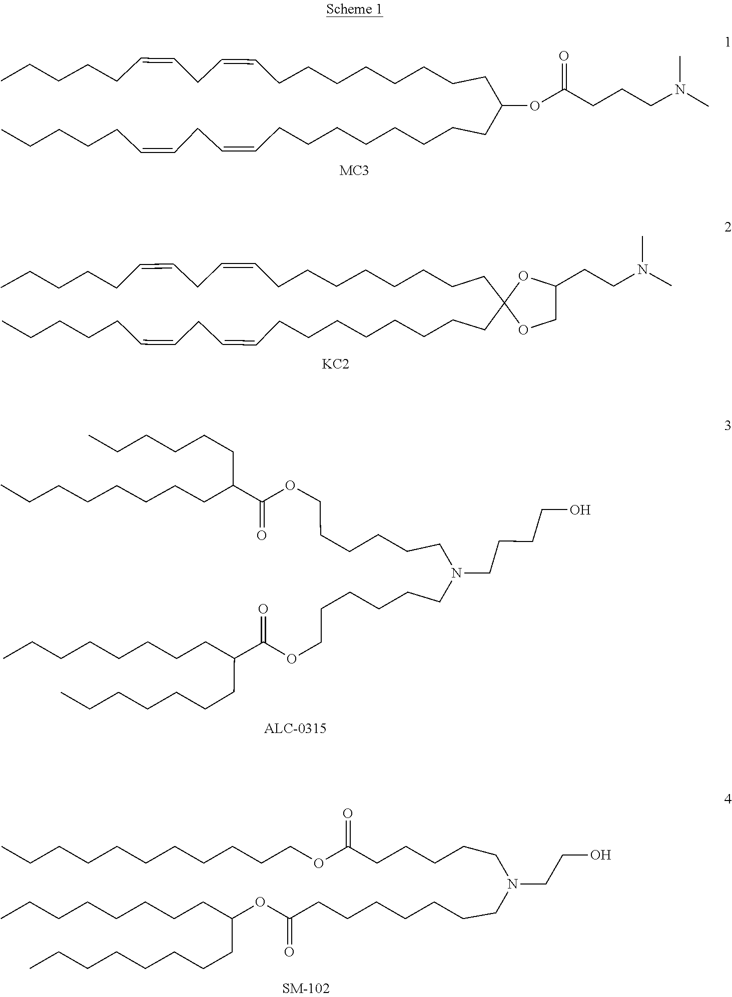

Furthermore, the above lipids were optimized for delivery of therapeutic nucleic acids to the liver. However, there is a need to develop new lipids for the delivery of charged cargo, such as nucleic acids to other organs, such as the spleen, lungs, bone marrow, skin, etc. The delivery of therapeutics beyond the liver would expand the clinical utility of LNPs to target disease conditions that affect tissues and organs beyond the liver. There is also an ongoing need to develop LNPs with improved delivery of nucleic acid or other charged cargo to the liver.

The present disclosure seeks to address one or more of the above-identified problems and/or provides useful alternatives to known products and/or compositions for the delivery of nucleic acid or other charged cargo.

### Definitions

As used herein, “type 1 ionizable head” or “MC-type ionizable head” refers to a moiety that has a head group of the lipid of Formula I below, or equivalents thereof, with n ranging from 1 to 5:

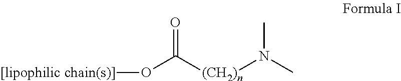

As used herein, “type 2 ionizable head” “KC-type ionizable head” refers to a moiety that has a head group of the lipid of Formula II below, or equivalents thereof, with n ranging from 1 to 5:

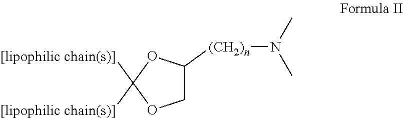

As used herein, “type 3 ionizable head” refers to a moiety that is the head group of the structure as defined by Formula III below, or equivalents thereof, with m and n independently ranging from 1 to 5:

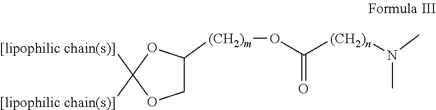

As used herein, “type 4 ionizable head” refers to a moiety that is the head group of the structure as defined by Formula IV below, or equivalents thereof, with m and n independently ranging from 2 to 5:

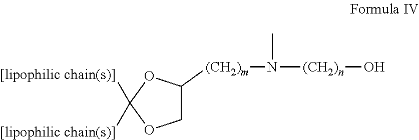

As used herein, “type 5 ionizable head” refers to a moiety that is the head group of the structure as defined by Formula V below, or equivalents thereof, with m and n independently ranging from 1 to 5:

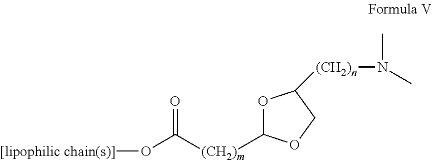

As used herein, “type 6 ionizable head” refers to a moiety that is the head group of the structure as defined by Formula VI below, or equivalents thereof, with m ranging from 1 to 5, and n independently ranging from 2 to 5:

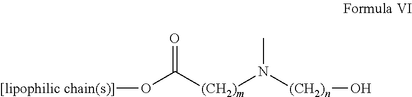

As used herein, “type 7 ionizable head” refers to a moiety that is the head group of the structure as defined by Formula VII below, or equivalents thereof, with n ranging from 1 to 5:

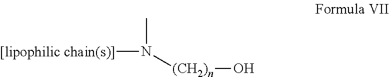

As used herein, “type 8 ionizable head” refers to a moiety that is the head group of the structure as defined by Formula VIII below, or equivalents thereof, with n ranging from 1 to 5:

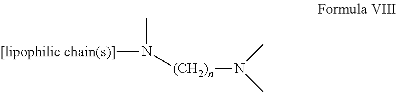

As used herein, “type 9 ionizable head” refers to a moiety that is the head group of the structure as defined by Formula IX below, or equivalents thereof, with m and n independently ranging from 1 to 5:

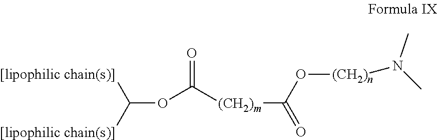

As used herein, the term “ionizable lipid” refers to a lipid that, at a given pH, is in an electrostatically neutral form and that may either accept or donate protons, thereby becoming electrostatically charged, and for which the electrostatically neutral form has a calculated logarithm of the partition coefficient between water and 1-octanol (i.e., a cLogP) that is greater than 8.

As used herein, the term “alkyl” or “alkyl group” as described herein is a carbon-containing chain that is linear or branched. The term is also meant to encompass a carbon-containing chain that optionally has varying degrees of unsaturation and that is optionally substituted.

As used herein, the term “Cₘ to Cₙ alkyl” or “Cₘ to Cₙ alkyl group” refers to a linear or branched carbon chain having a total minimum of m carbon atoms and up to n carbon atoms, and that is optionally unsaturated and optionally substituted. For example, a “C₁ to C₃ alkyl” or “C₁ to C₃ alkyl group” is an alkyl having between 1 and 3 carbon atoms.

The term “optionally substituted” with reference to an alkyl means that at least one hydrogen atom of the alkyl group can be replaced by a non-hydrogen atom or group of atoms (i.e., a “substituent”), and/or the alkyl is interrupted by one or more substituents comprising heteroatoms selected from O, S and NR′, wherein R′ is as defined below. Non-limiting examples of groups that may replace a hydrogen atom include halogen; alkyl groups; cycloalkyl groups; oxo groups (═O); hydroxyl groups (—OH); (C═O)OR′; O(C═O)R′; C(═O)R′; OR′; S(O)ₓR′; S—SR′; C(═O)SR′; SC(═O)R′; NR′R′; NR′C(═O)R′; C(═O)NR′R′; NR′C(═O)NR′R′; OC(═O)NR′R′; NR′C(═O)OR′; NR'S(O)ₓNR′R′; NR′S(O)ₓR′; and S(O)ₓNR′R′, wherein R′ at each occurrence is independently selected from H, C₁-C₁₅ alkyl or cycloalkyl, and x is 0, 1 or 2.

As used herein, the term “lipophilic chain” refers to an alkyl group bonded to a nitrogen or carbon atom of the lipid, said alkyl group comprising at least 6 C atoms and optionally comprising C═C double bonds, and/or ring structures, and/or carbonyl groups, and/or heteroatoms such as N, O, S, and such that the parent compound of said alkyl group has a CLogP of at least 6.

For example, lipid MC3, 1, and lipid KC2, 2, have a pair of lipophilic chains derived from (6Z,9Z)-octadeca-6,9-diene, which has a CLogP of 9.25:

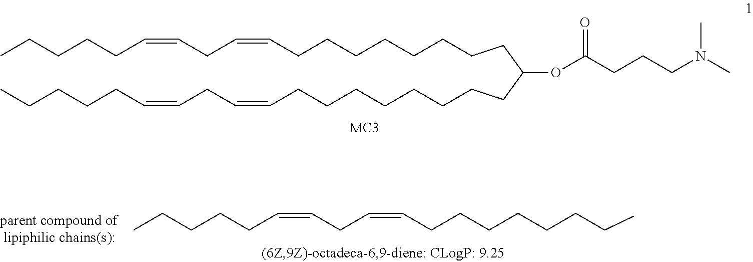

Lipid ALC-0315, 3, has a pair of lipophilic chains derived from hexyl 2-hexyldecanoate, which has a CLogP of 10.01:

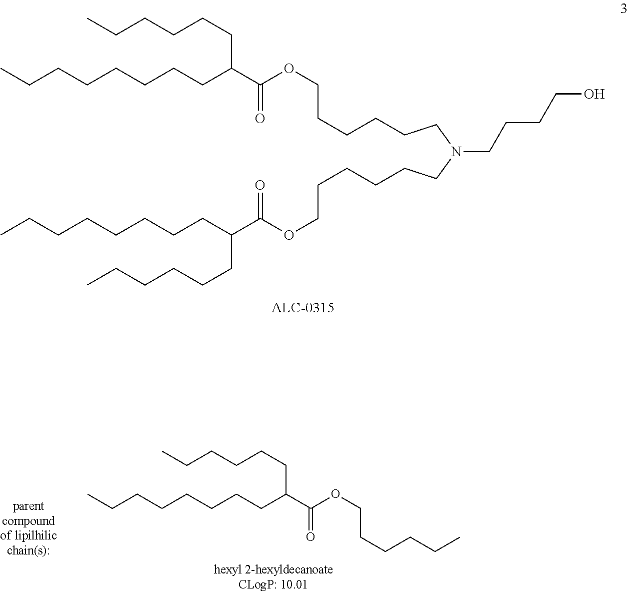 [SMILES: CCCCCCCCC(CCCCCC)C(=O)OCCCCCCN(CCCCO)CCCCCCOC(=O)C(CCCCCC)CCCCCCCC ; CCCCCCCCC(CCCCCC)C(=O)OCCCCCC]

Lipid SM-102, 4, has one lipophilic chain derived from undecyl hexanoate, which has a CLogP of 7.59, and one lipophilic chain derived from heptadecane-9-yl octanoate, which has a CLogP of 11.6:

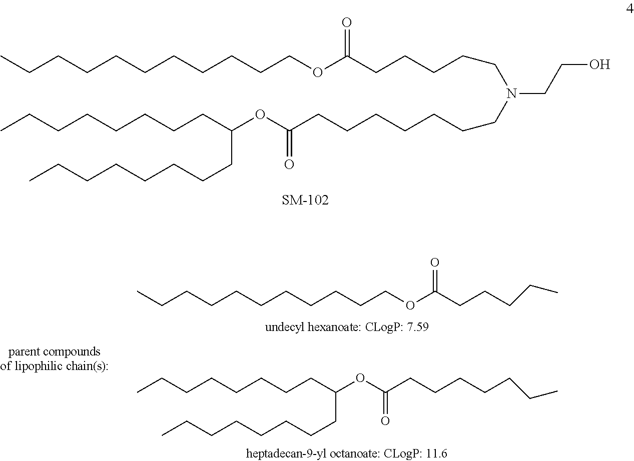

As used herein, the term “helper lipid” means a compound selected from: a sterol such as cholesterol or a derivative thereof, a diacylglycerol or a derivative thereof, such as a glycerophospholipid, including phosphatidic acid (phosphatidate) (PA), phosphatidylethanolamine (cephalin) (PE), phosphatidylcholine (PC), phosphatidylserine (PS), and the like; and a sphingolipid, such as a ceramide, a sphingomyelin, a cerebroside, a ganglioside, or reduced analogues thereof, that lack a double bond in the sphingosine unit. An example of a diacylglycerol derivative is a glycerophospholipid-cholesterol conjugate in which one of the acyl chains is substituted with a moiety comprising cholesterol. The term encompasses lipids that are either naturally-occurring or synthetic.

As used herein, the term “delivery vehicle” includes any preparation in which the lipid described herein is capable of being formulated and includes but is not limited to delivery vehicles comprising helper lipids.

As used herein, the term “nanoparticle” is any suitable particle in which the lipid can be formulated and that may comprise one or more helper lipid components. The one or more lipid components may include an ionizable lipid prepared by the method described herein and/or may include additional lipid components, such as the one or more helper lipid components. The term includes, but is not limited to, vesicles with one or more bilayers, including multilamellar vesicles, unilamellar vesicles and vesicles with an electron-dense core. The term also includes polymer-lipid hybrids, including particles in which the lipid is attached to a polymer.

As used herein, the term “encapsulated,” with reference to incorporating a cargo molecule (e.g., mRNA) within a delivery vehicle refers to any association of the cargo with any component or compartment of the delivery vehicle such as a nanoparticle.

The term “pharmaceutically acceptable salt” with reference to a form of the lipid of the disclosure in a protonated form (i.e., charged) and/or as part of a pharmaceutical formulation in which an LNP is formulated refers to a salt of the lipid prepared from pharmaceutically acceptable acids, including inorganic and organic acids.

The term “sarcosine group” or a derivative thereof as used within this specification includes a group having the formula:

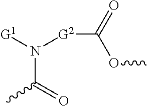

wherein the wavy lines represent respective covalent bonds to atoms within the lipophilic chains. The sarcosine group and derivatives thereof can be present in a lipophilic chain of a lipid in any orientation. For example, the terminal oxygen of the ester moiety (—C(O)O—) can be covalently bonded to an alkylene group (e.g., (CH₂)ₙ wherein n is 4-8) that links the head group moiety of a lipid to the sarcosine group and the carbonyl of the amide group (—NC(O)—) covalently bonded to a terminal alkyl group of the lipophilic chain of the lipid. G¹ and G² are as defined herein in connection with Formula B and Formula D set forth below.

The term “proline group” or a derivative thereof as used within this specification includes a group having the formula:

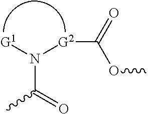

wherein the wavy lines represent respective covalent bonds to atoms within the lipophilic chains. The solid semicircle between G¹ and G² denotes that a first atom that is part of G¹ is bonded to a second atom that is part of G² so as to form a ring structure, such as a 5- or 6-membered ring, that includes the N atom. The proline group and derivatives thereof can be present in a lipophilic chain of a lipid in any orientation. For example, the terminal oxygen of the ester moiety (—C(O)O—) can be covalently bonded to an alkylene group (e.g., (CH₂)ₙ wherein n is 4-8) that links the head group moiety of a lipid to the proline group and the carbonyl of the amide group (—NC(O)—) covalently bonded to a terminal alkyl group of the lipophilic chain of the lipid. G¹ and G² are as defined herein in connection with Formula B and Formula D set forth below.

The article “a” or “an” as used herein is meant to include both singular and plural, unless otherwise indicated.

### SUMMARY

The present disclosure is based, at least in part, on the surprising discovery that LNP formulations of nucleic acid, such as messenger RNA (mRNA), comprising ionizable lipids that incorporate certain amino acid moieties in their lipophilic chains are more potent than the benchmark MC3, ALC-0315 or SM-102 for liver delivery of nucleic acid, such as RNA. In some embodiments, the lipids of the disclosure exhibit a different organ selectivity relative to known lipids. In particular, certain embodiments described herein promote delivery of nucleic acid, such as mRNA, selectively to extrahepatic tissues or organs, such as the spleen, more efficiently than known lipids. In addition, the chemical synthesis of the lipids of certain embodiments herein is more straightforward and/or economical than that of known lipids.

According to one embodiment of the disclosure, there is provided a lipid having the structure of Formula A:

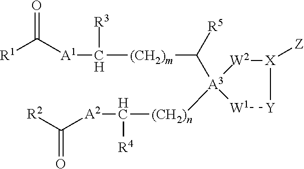

or a pharmaceutically acceptable salt thereof,
wherein indices m and n vary, independently, from 1 to 8;
groups A¹ and A² are both present or both absent, and
if groups A¹ and A² are both present, they are both O, and
at least one of R¹ and R² is an amino acid-derived moiety having the structure Formula B

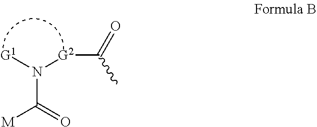

wherein the wavy bond connects to A¹ and/or A² (that is, O) in Formula A,
G¹ is a C₁-C₆ small alkyl or cycloalkyl, optionally comprising one or more heteroatoms such as N, O, S;
G² is (CR^{a}R^{b})ₚ, wherein R^{a} and R^{b} are, independently, H or small C₁-C₆ alkyl or cycloalkyl, and index p can range from p=1 to p=5;
the dotted semicircle between G¹ and G² signifies that one of the atoms that are part of G¹ may be bonded to one of the atoms that are part of G², so as to create a ring structure that encompasses the N atom;
M is optionally
a linear or branched alkyl group having from 10 to 20 carbon atoms; optionally comprising from 1 to 3 C═C double bonds of E or Z geometry; optionally comprising 1 to 3 heteroatoms such as N, O, S, which are optionally part of a functional group that is an ester, ether, amide, amine or thioether; and optionally comprising additional OH, O-alkyl, S-alkyl substituents, or
a moiety of Formula C,

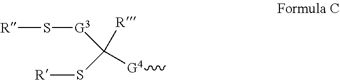

wherein R′ and R″ are, independently, a linear or branched alkyl group comprising from 3 to 12 C atoms, optionally incorporating from 1 to 3 C═C double bonds of E or Z geometry; optionally incorporating 1 to 3 heteroatoms such as N, O, S, which are optionally part of a functional group that is an ester, ether, amide, amine or thioether; and optionally incorporating additional OH, O-alkyl, S-alkyl substituents;
R′″ is H or a linear, branched, or cyclic alkyl group comprising from 1 to 6 C atoms, and optionally incorporating heteroatoms such as N, O, S;
G³ and G⁴ are, independently, a group of structure (CR^{a}R^{b})_{q} with R^{a} and R^{b} independently equal to H or small C₁-C₅ alkyl or cycloalkyl, and with index q varying from q=0 (in which case G³/G⁴ is/are absent) to q=6, and
If only one of R¹ and R² is a group as described above, then the other one of R¹ and R² is a linear or branched or cyclic alkyl group comprising from 4 to 30 C atoms, optionally comprising heteroatoms such as N, O, S, optionally comprising from 1 to 3 C═C double bonds of E or Z geometry, optionally comprising substituents such as alkyl or cycloalkyl, O-alkyl, S-alkyl, wherein the alkyl or cycloalkyl or O-alkyl or S-alkyl group comprises from 1 to 8 carbon atoms,
R³ and R⁴ are, independently, H or a group of structure R⁶—S—CH₂, wherein R⁶ is a C₁-C₁₂ alkyl or cycloalkyl group, and
R⁵ is H or a C₁-C₆ alkyl group optionally comprising a C═C double bond of E or Z geometry, optionally comprising up to 3 heteroatoms such as N, O, S.

If groups A¹ and A² are both absent, then
R³ and R⁴ are both H
R⁵ is H or a C₁-C₆ alkyl group optionally comprising a C═C double bond of E or Z geometry, optionally comprising up to 3 heteroatoms such as N, O, S,
at least one of R¹ and R² is an amino acid-derived moiety as depicted in Formula D,

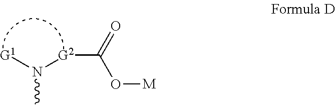

wherein the wavy bond connects the N atom to the C═O group in Formula A,
G¹ is a C₁-C₆ small alkyl or cycloalkyl, optionally comprising one or more heteroatoms such as N, O, S;
G² is (CR^{a}R^{b})ₚ, wherein R^{a} and R^{b} are, independently, H or small C₁-C₆ alkyl or cycloalkyl, and index p can range from p=1 to p=5;
the dotted semicircle between G¹ and G² signifies that one of the atoms that are part of G¹ may be bonded to one of the atoms that are part of G², so as to create a ring structure that encompasses the N atom;
M is a linear or branched alkyl group having from 10 to 20 carbon atoms; optionally comprising from 1 to 3 C═C double bonds of E or Z geometry; optionally comprising 1 to 3 heteroatoms such as N, O, S, which may be part of a functional group that is an ester, ether, amide, amine or thioether; and optionally incorporating additional OH, O-alkyl, S-alkyl substituents, or
a moiety of Formula C,

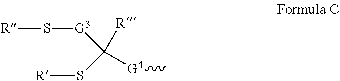

wherein R′, R″, R′″, G³ and G⁴ are as defined above.

If only one of R¹ and R² is a group as described above, then the other one of R¹ and R² is a group of the type R^{c}—O, wherein the O atom is bound to the C═O group in Formula A, and R^{c} is a linear or branched or cyclic alkyl group comprising from 4 to 30 C atoms, optionally comprising heteroatoms such as N, O, S, optionally comprising from 1 to 3 C═C double bonds of E or Z geometry, optionally comprising substituents such as alkyl or cycloalkyl, O-alkyl, S-alkyl, wherein the alkyl or cycloalkyl or O-alkyl or S-alkyl group comprises from 1 to 8 carbon atoms.

A² is either C or N, and if A² is C, then
R⁵ is H or a C₁-C₅ alkyl
W¹ and Y are either bonded to each other or not bonded to each other (as indicated by the dashed bond), and:
   if W and Y are bonded to each other then:
      W¹ is O or S;
      W² is O or S;
      X is CH;
      Y is (CH₂)ₚ, wherein p is 1 or 2;
      Z is a group chosen from among structures a-c below, wherein the wavy line represents the bond to X

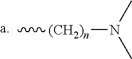

type 2 ionizable head;

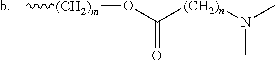

type 3 ionizable head;

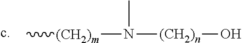

type 4 ionizable head,
if W and Y are not bonded to each other, then:
   W¹ is H;
   W² is O or S or NH or NR², wherein R² is a C₁ to C₄ small alkyl optionally substituted with an OH group;
Group

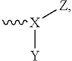

wherein the wavy line represents the bond to W², is a group chosen from among structures d-i below, wherein the wavy line represents the bond to W²:

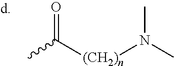

if W² is O, type 1 ionizable head;

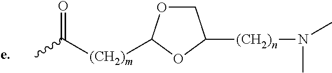

if W² is O, type 5 ionizable head;

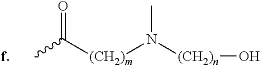

if W² is O, type 6 ionizable head;

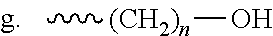

if W² is NH or NR², type 7 ionizable head;

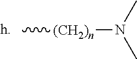

if W² is NH or NR², type 8 ionizable head; and

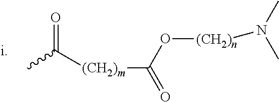

if W² is O, type 9 ionizable head;
   if A³ is N, then:
R⁵ is H or (CH₂)_{q}—OH, wherein index q ranges from q=2 to q=8,
W¹ and Y are absent,
W², X and Z together form a group of structure (CR^{a}R^{b})ₜ-A³ with index t varying from t=1 to t=6, R^{a} and R^{b} independently equal to H or small C₁-C₅ alkyl or cycloalkyl, and with Z equal to H, or OH, or NR′R″, wherein R′ and R″ are small C₁-C₅ alkyls or cycloalkyls, or wherein R′ and R″ are branches of a heterocyclic group that incorporates the N atom to which R′ and R″ are bound, such as pyrrolidine, piperidine, morpholine, and the like.

According to one embodiment of the disclosure, each lipophilic chain in the lipid of Formula A has between 12 and 30 carbon atoms in total.

According to another embodiment of the disclosure, the lipid of Formula A has (i) a pKₐ of between 6 and 8; and (ii) a ClogP of at least 10.

According to any one of the foregoing aspects or embodiments, the lipid of Formula A, when formulated in a lipid nanoparticle comprising an mRNA, results in an increase in biodistribution of the lipid nanoparticle in a particular organ, such as the liver or one or more extrahepatic tissues, of at least about 10% relative to a lipid nanoparticle containing DLin-MC3-DMA (1), ALC-0315 (3) or SM-102 (4) as measured by luminescence of the mRNA in vivo in the liver and/or the one or more extrahepatic tissues. The assay and the formulations used to determine the biodistribution are as described in Example 2.

In another aspect, there is provided a lipid nanoparticle comprising the lipid of any one of the aspects or embodiments described above and a nucleic acid.

The lipid nanoparticle may comprise a helper lipid and a hydrophilic polymer-lipid conjugate. The helper lipid may be selected from cholesterol, a diacylglycerol and a sphingolipid.

In another aspect, there is provided a lipid nanoparticle comprising:
an ionizable lipid with two lipophilic chains, at least one of the chains comprising an amino acid-derived moiety selected from a sarcosine group and a proline group or derivatives thereof in one or both of the lipophilic chains;
one or more helper lipids;
optionally a hydrophilic polymer-lipid conjugate; and
a nucleic acid.

In a further aspect, there is provided a method for administering a nucleic acid to a subject in need thereof, the method comprising preparing or providing the lipid nanoparticle as described in any of the foregoing aspects or embodiments comprising the nucleic acid and administering the lipid nanoparticle to the subject.

According to another aspect of the disclosure, there is provided a method for delivering a cargo molecule to a cell, the method comprising contacting the lipid nanoparticle as described in any aspect or embodiment as described above with the cell in vivo or in vitro. In one embodiment, the cargo molecule is a nucleic acid.

According to a further aspect of the disclosure, there is provided a use of the lipid or a pharmaceutically acceptable salt thereof as defined above or the lipid nanoparticle as defined in any aspect or embodiment described above, in the manufacture of a medicament to treat or prevent a disease, disorder or condition that is treatable and/or preventable by a nucleic acid.

According to a further aspect of the disclosure, there is provided a use of the lipid or the pharmaceutically acceptable salt thereof as defined above or the lipid nanoparticle of any one of the aspects or embodiments described above to deliver a nucleic acid to a patient to treat or prevent a disease, disorder or condition that is treatable or preventable by the nucleic acid. In one embodiment, the nucleic acid is an mRNA.

Other objects, features, and advantages of the present disclosure will be apparent to those of skill in the art from the following detailed description and figures.

### BRIEF DESCRIPTION OF THE DRAWINGS

FIG. 1 is a bar graph showing entrapment (%), particle size, and polydispersity index (PDI) of mRNA-containing lipid nanoparticles (LNPs) comprising the ionizable lipids 1, 5-7, 16-19, 21-27 and 31-36. The LNPs are composed of 50/10/38.5/1.5 mol % of ionizable lipid/DSPC/chol/PEG-DMG and the amine-to-phosphate (N/P) was 6.

FIG. 2A shows luminescence intensity/mg in the liver for the mRNA-containing LNPs comprising the ionizable lipids 1, 3-7, 16-19, 21-27 and 31-36 measured 4 hours post-intravenous administration to CD-1 mice. The LNPs contain 50/10/38.5/1.5 mol % of ionizable lipid/DSPC/chol/PEG-DMG (N/P=6).

FIG. 2B shows luminescence intensity/mg in the spleen for the mRNA-containing LNPs comprising the ionizable lipids 1, 3-7, 16-19, 21-27 and 31-36 measured 4 hours post-intravenous administration to CD-1 mice. The LNPs contain 50/10/38.5/1.5 mol % of ionizable lipid/DSPC/chol/PEG-DMG (N/P=6).

FIG. 3A shows liver selectivity for the mRNA-containing LNPs comprising ionizable lipids 1, 3-7, 16-19, 21-27 and 31-36 measured 4 hours post-intravenous administration to CD-1 mice. The data is plotted as activity for each lipid relative to lipid 1 (MC3). The LNPs contain 50/10/38.5/1.5 mol % of ionizable lipid/DSPC/chol/PEG-DMG (N/P=6).

FIG. 3B shows spleen selectivity for the mRNA-containing LNPs comprising ionizable lipids 1, 3-7, 16-19, 21-27 and 31-36 measured 4 hours post-intravenous administration to CD-1 mice. The data is plotted as activity for each lipid relative to lipid 1 (MC3). The LNPs contain 50/10/38.5/1.5 mol % of ionizable lipid/DSPC/chol/PEG-DMG (N/P=6).

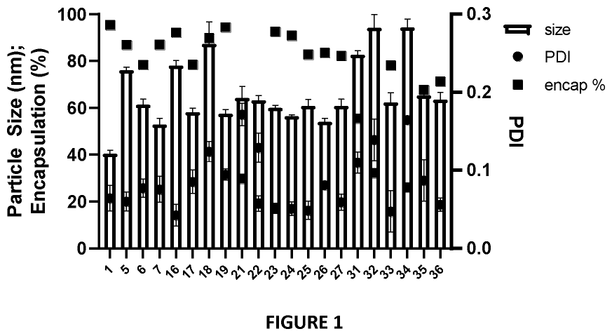

### DETAILED DESCRIPTION

Various aspects and embodiments of the disclosure are directed to ionizable lipids having structures of Formula A. Formulations comprising such lipids find use in the delivery of nucleic acid to any target site. In some embodiments, such lipids have been found to be particularly efficacious for the delivery of mRNA when formulated in a suitable delivery vehicle. In further embodiments, such lipids can be easily synthesized and prepared by processes having improved economics relative to known methods for making ionizable lipids.

Representative, but by no means limiting, examples of lipids of Formula A are compounds 4-45 of Table 1 below.

TABLE 1

| cpd. | |
|---|---|
| no. | structure |
| 5 | 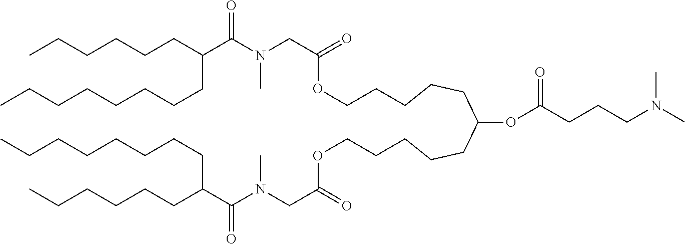 [SMILES: CCCCCCCCC(CCCCCC)C(=O)N(C)CC(=O)OCCCCCC(CCCCCOC(=O)CN(C)C(=O)C(CCCCCC)CCCCCCCC)OC(=O)CCCN(C)C] |
| 6 | 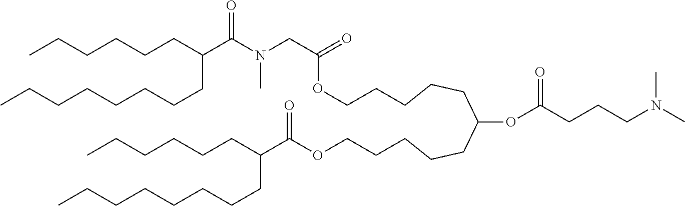 [SMILES: CCCCCCCCC(CCCCCC)C(=O)OCCCCCC(CCCCCOC(=O)CN(C)C(=O)C(CCCCCC)CCCCCCCC)OC(=O)CCCN(C)C] |
| 7 | 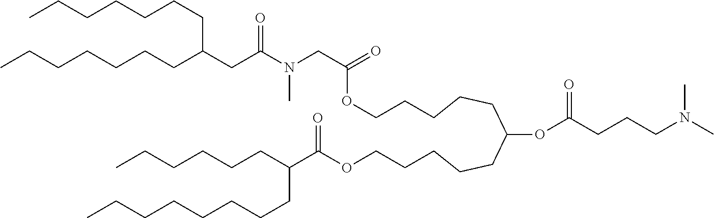 [SMILES: CCCCCCCCC(CCCCCC)C(=O)OCCCCCC(CCCCCOC(=O)CN(C)C(=O)CC(CCCCCCC)CCCCCCC)OC(=O)CCCN(C)C] |
| 8 | 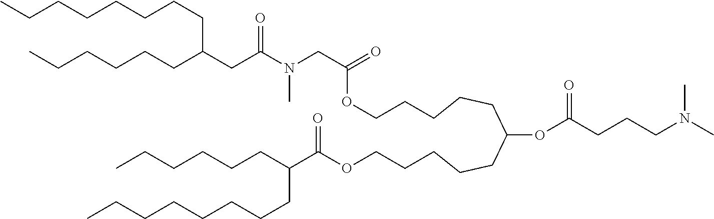 [SMILES: CCCCCCCCC(CCCCCC)CC(=O)N(C)CC(=O)OCCCCCC(CCCCCOC(=O)C(CCCCCC)CCCCCCCC)OC(=O)CCCN(C)C] |
| 9 | 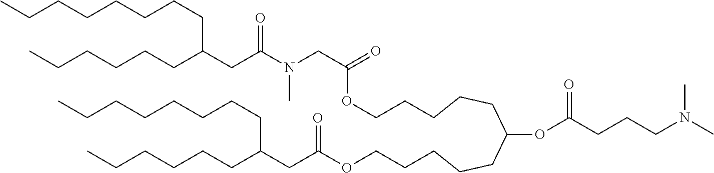 [SMILES: CCCCCCCCC(CCCCCC)CC(=O)OCCCCCC(CCCCCOC(=O)CN(C)C(=O)CC(CCCCCC)CCCCCCCC)OC(=O)CCCN(C)C] |
| 10 | 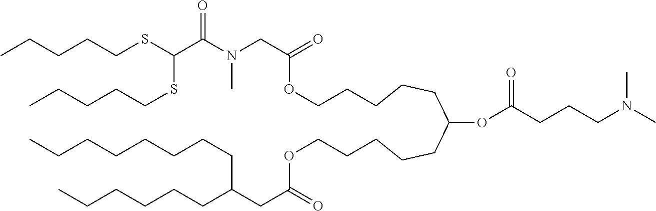 [SMILES: CCCCCCCCC(CCCCCC)CC(=O)OCCCCCC(CCCCCOC(=O)CN(C)C(=O)C(SCCCCC)SCCCCC)OC(=O)CCCN(C)C] |
| 11 | 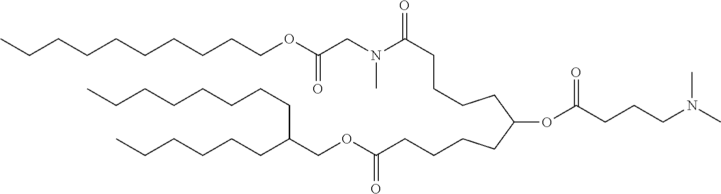 [SMILES: CCCCCCCCCCOC(=O)CN(C)C(=O)CCCCC(CCCCC(=O)OCC(CCCCCC)CCCCCCCC)OC(=O)CCCN(C)C] |
| 12 | 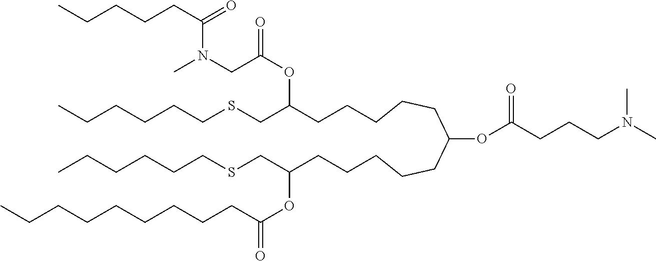 [SMILES: CCCCCCCCCC(=O)OC(CCCCCC(CCCCCC(CSCCCCCC)OC(=O)CN(C)C(=O)CCCCC)OC(=O)CCCN(C)C)CSCCCCCC] |
| 13 | 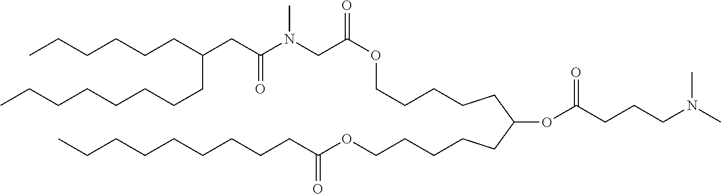 [SMILES: CCCCCCCCCC(=O)OCCCCCC(CCCCCOC(=O)CN(C)C(=O)CC(CCCCCC)CCCCCCCC)OC(=O)CCCN(C)C] |
| 14 | 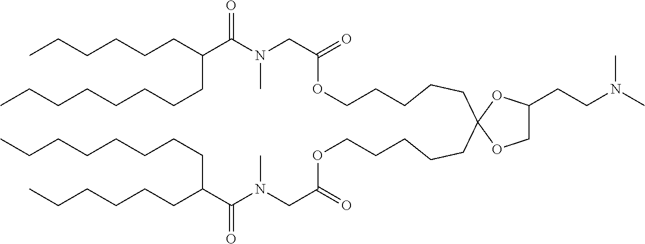 [SMILES: CCCCCCCCC(CCCCCC)C(=O)N(C)CC(=O)OCCCCCC1(CCCCCOC(=O)CN(C)C(=O)C(CCCCCC)CCCCCCCC)OCC(CCN(C)C)O1] |
| 15 | 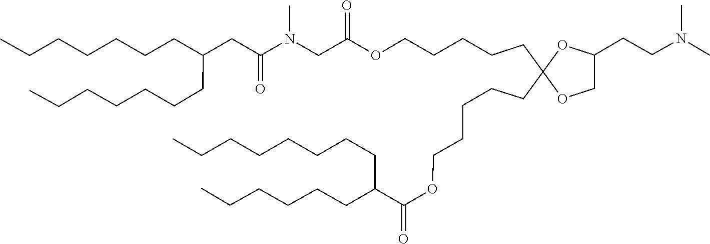 [SMILES: CCCCCCCCC(CCCCCC)C(=O)OCCCCCC1(CCCCCOC(=O)CN(C)C(=O)CC(CCCCCCC)CCCCCCC)OCC(CCN(C)C)O1] |
| 16 | 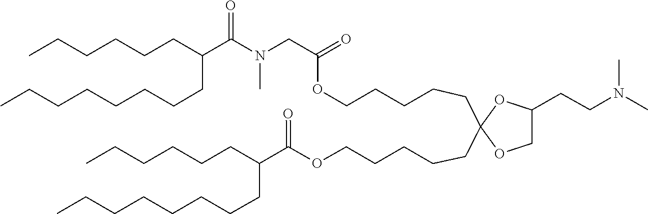 [SMILES: CCCCCCCCC(CCCCCC)C(=O)OCCCCCC1(CCCCCOC(=O)CN(C)C(=O)C(CCCCCC)CCCCCCCC)OCC(CCN(C)C)O1] |
| 17 | 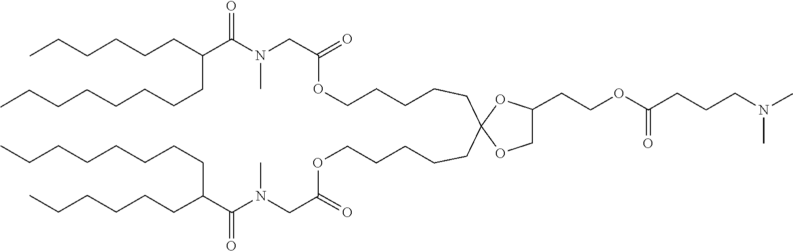 [SMILES: CCCCCCCCC(CCCCCC)C(=O)N(C)CC(=O)OCCCCCC1(CCCCCOC(=O)CN(C)C(=O)C(CCCCCC)CCCCCCCC)OCC(CCOC(=O)CCCN(C)C)O1] |
| 18 | 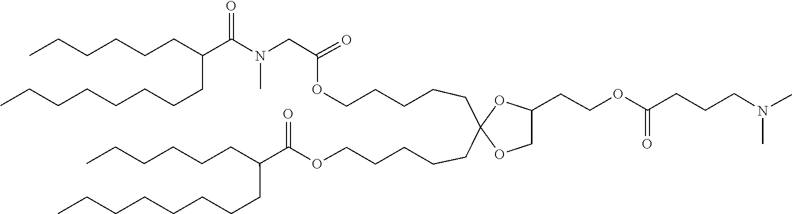 [SMILES: CCCCCCCCC(CCCCCC)C(=O)OCCCCCC1(CCCCCOC(=O)CN(C)C(=O)C(CCCCCC)CCCCCCCC)OCC(CCOC(=O)CCCN(C)C)O1] |
| 19 | 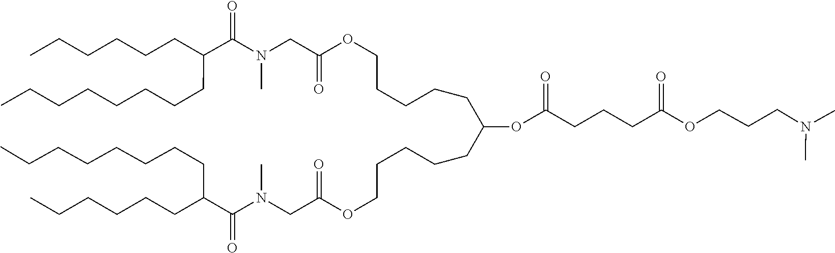 [SMILES: CCCCCCCCC(CCCCCC)C(=O)N(C)CC(=O)OCCCCCC(CCCCCOC(=O)CN(C)C(=O)C(CCCCCC)CCCCCCCC)OC(=O)CCCC(=O)OCCCN(C)C] |
| 20 | 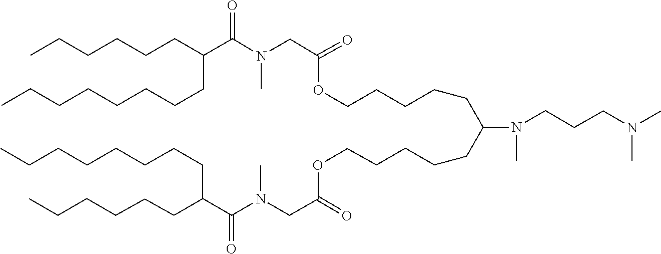 [SMILES: CCCCCCCCC(CCCCCC)C(=O)N(C)CC(=O)OCCCCCC(CCCCCOC(=O)CN(C)C(=O)C(CCCCCC)CCCCCCCC)N(C)CCCN(C)C] |
| 21 | 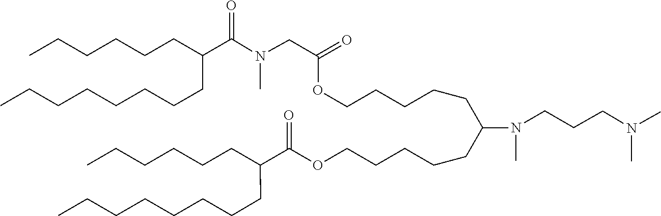 [SMILES: CCCCCCCCC(CCCCCC)C(=O)OCCCCCC(CCCCCOC(=O)CN(C)C(=O)C(CCCCCC)CCCCCCCC)N(C)CCCN(C)C] |
| 22 | 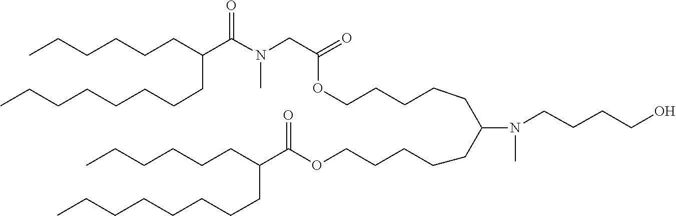 [SMILES: CCCCCCCCC(CCCCCC)C(=O)OCCCCCC(CCCCCOC(=O)CN(C)C(=O)C(CCCCCC)CCCCCCCC)N(C)CCCCO] |
| 23 | 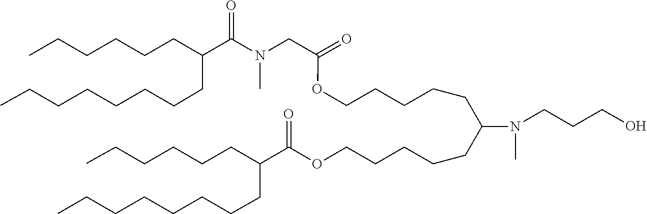 [SMILES: CCCCCCCCC(CCCCCC)C(=O)OCCCCCC(CCCCCOC(=O)CN(C)C(=O)C(CCCCCC)CCCCCCCC)N(C)CCCO] |
| 24 | 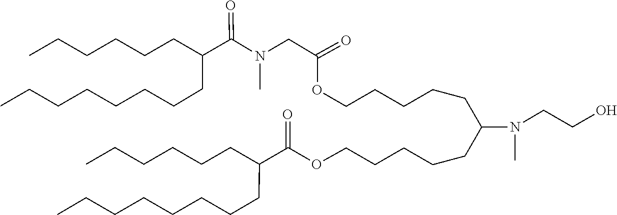 [SMILES: CCCCCCCCC(CCCCCC)C(=O)OCCCCCC(CCCCCOC(=O)CN(C)C(=O)C(CCCCCC)CCCCCCCC)N(C)CCO] |
| 25 | 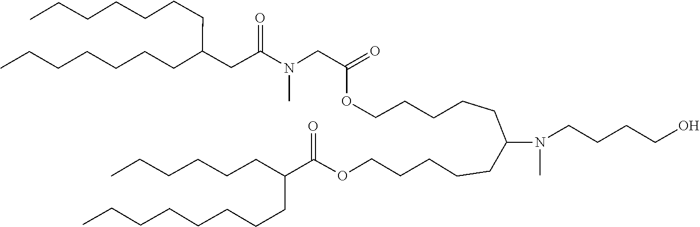 [SMILES: CCCCCCCCC(CCCCCC)C(=O)OCCCCCC(CCCCCOC(=O)CN(C)C(=O)CC(CCCCCCC)CCCCCCC)N(C)CCCCO] |
| 26 | 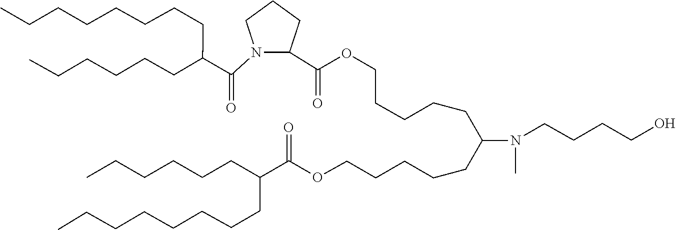 [SMILES: CCCCCCCCC(CCCCCC)C(=O)OCCCCCC(CCCCCOC(=O)C1CCCN1C(=O)C(CCCCCC)CCCCCCCC)N(C)CCCCO] |
| 27 | 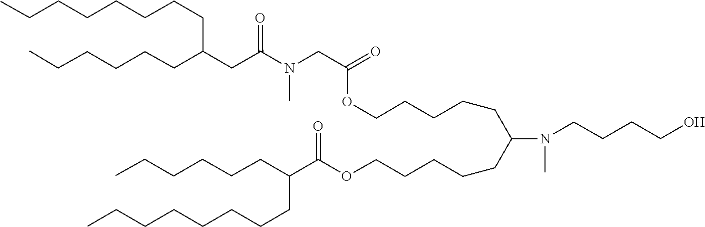 [SMILES: CCCCCCCCC(CCCCCC)CC(=O)N(C)CC(=O)OCCCCCC(CCCCCOC(=O)C(CCCCCC)CCCCCCCC)N(C)CCCCO] |
| 28 | 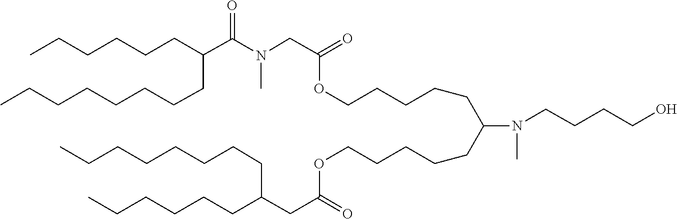 [SMILES: CCCCCCCCC(CCCCCC)CC(=O)OCCCCCC(CCCCCOC(=O)CN(C)C(=O)C(CCCCCC)CCCCCCCC)N(C)CCCCO] |
| 29 | 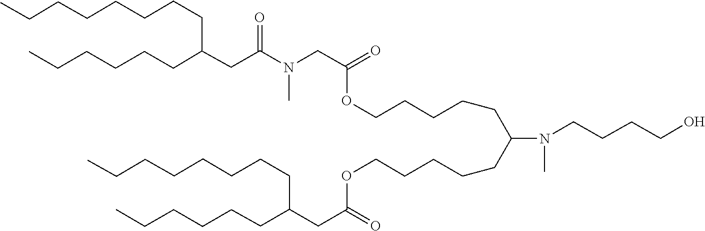 [SMILES: CCCCCCCCC(CCCCCC)CC(=O)OCCCCCC(CCCCCOC(=O)CN(C)C(=O)CC(CCCCCC)CCCCCCCC)N(C)CCCCO] |
| 30 | 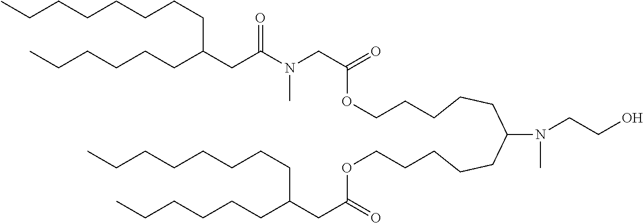 [SMILES: CCCCCCCCC(CCCCCC)CC(=O)OCCCCCC(CCCCCOC(=O)CN(C)C(=O)CC(CCCCCC)CCCCCCCC)N(C)CCO] |
| 31 | 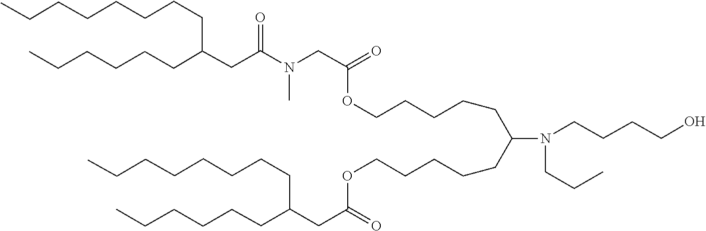 [SMILES: CCCCCCCCC(CCCCCC)CC(=O)OCCCCCC(CCCCCOC(=O)CN(C)C(=O)CC(CCCCCC)CCCCCCCC)N(CCC)CCCCO] |
| 32 | 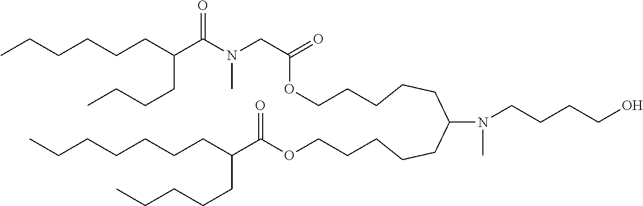 [SMILES: CCCCCCCC(CCCCC)C(=O)OCCCCCC(CCCCCOC(=O)CN(C)C(=O)C(CCCC)CCCCCC)N(C)CCCCO] |
| 33 | 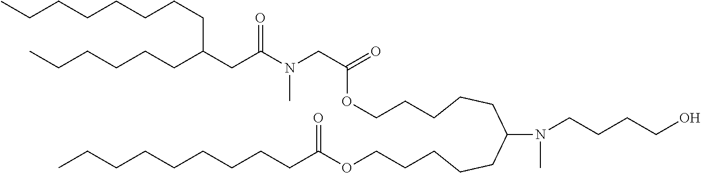 [SMILES: CCCCCCCCCC(=O)OCCCCCC(CCCCCOC(=O)CN(C)C(=O)CC(CCCCCC)CCCCCCCC)N(C)CCCCO] |
| 34 | 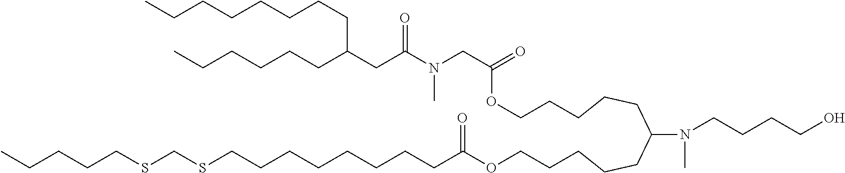 [SMILES: CCCCCCCCC(CCCCCC)CC(=O)N(C)CC(=O)OCCCCCC(CCCCCOC(=O)CCCCCCCCSCSCCCCC)N(C)CCCCO] |
| 35 | 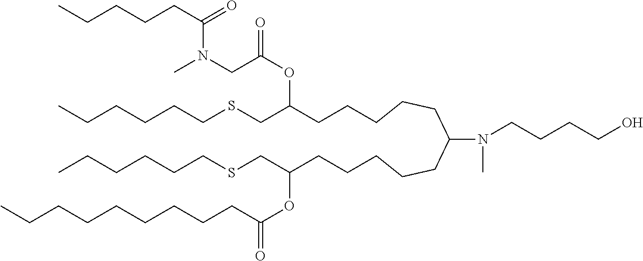 [SMILES: CCCCCCCCCC(=O)OC(CCCCCC(CCCCCC(CSCCCCCC)OC(=O)CN(C)C(=O)CCCCC)N(C)CCCCO)CSCCCCCC] |
| 36 | 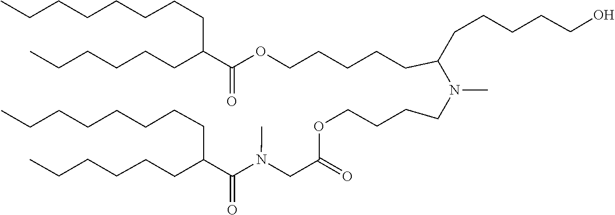 [SMILES: CCCCCCCCC(CCCCCC)C(=O)OCCCCCC(CCCCCO)N(C)CCCCOC(=O)CN(C)C(=O)C(CCCCCC)CCCCCCCC] |
| 37 | 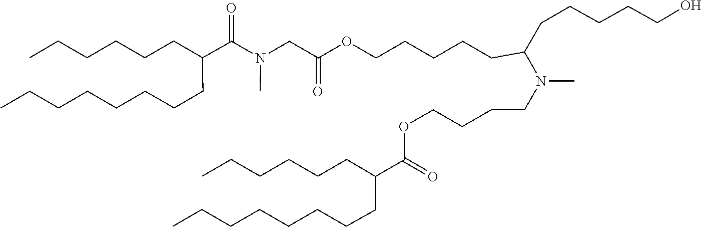 [SMILES: CCCCCCCCC(CCCCCC)C(=O)OCCCCN(C)C(CCCCCO)CCCCCOC(=O)CN(C)C(=O)C(CCCCCC)CCCCCCCC] |
| 38 | 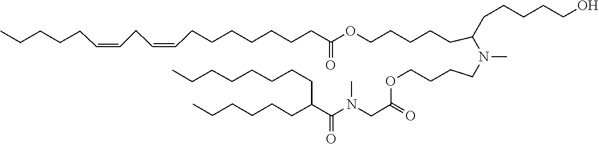 [SMILES: CCCCC/C=C\C/C=C\CCCCCCCC(=O)OCCCCCC(CCCCCO)N(C)CCCCOC(=O)CN(C)C(=O)C(CCCCCC)CCCCCCCC] |
| 39 | 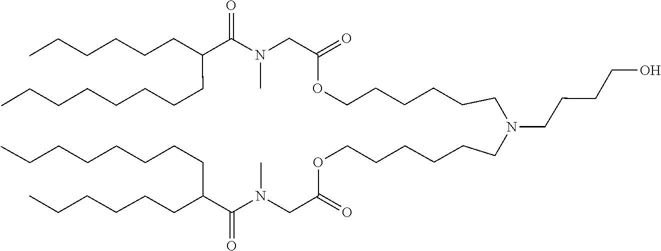 [SMILES: CCCCCCCCC(CCCCCC)C(=O)N(C)CC(=O)OCCCCCCN(CCCCO)CCCCCCOC(=O)CN(C)C(=O)C(CCCCCC)CCCCCCCC] |
| 40 | 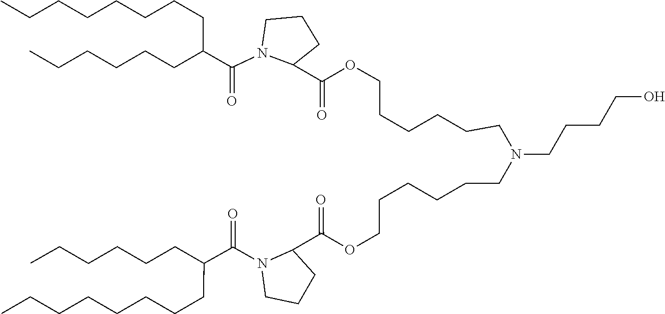 [SMILES: CCCCCCCCC(CCCCCC)C(=O)N1CCCC1C(=O)OCCCCCCN(CCCCO)CCCCCCOC(=O)C1CCCN1C(=O)C(CCCCCC)CCCCCCCC] |
| 41 | 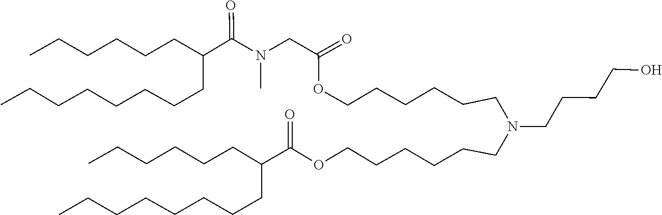 [SMILES: CCCCCCCCC(CCCCCC)C(=O)OCCCCCCN(CCCCO)CCCCCCOC(=O)CN(C)C(=O)C(CCCCCC)CCCCCCCC] |
| 42 | 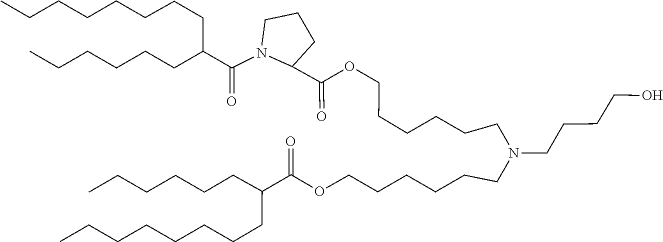 [SMILES: CCCCCCCCC(CCCCCC)C(=O)OCCCCCCN(CCCCO)CCCCCCOC(=O)C1CCCN1C(=O)C(CCCCCC)CCCCCCCC] |
| 43 | 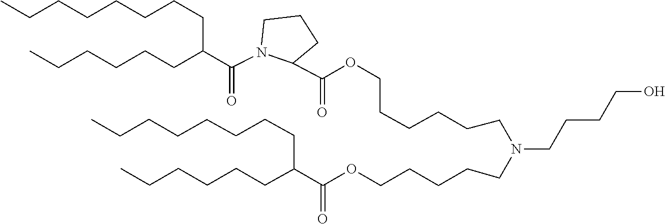 [SMILES: CCCCCCCCC(CCCCCC)C(=O)OCCCCCN(CCCCO)CCCCCCOC(=O)C1CCCN1C(=O)C(CCCCCC)CCCCCCCC] |
| 44 | 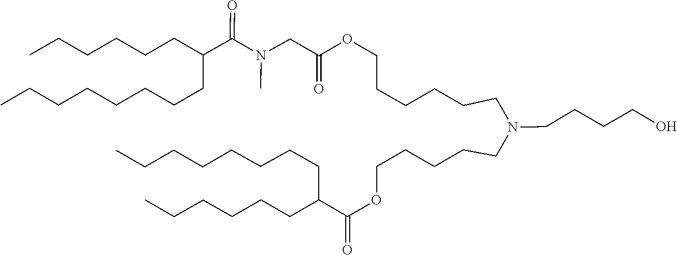 [SMILES: CCCCCCCCC(CCCCCC)C(=O)OCCCCCN(CCCCO)CCCCCCOC(=O)CN(C)C(=O)C(CCCCCC)CCCCCCCC] |

### Methods to Produce Lipids of Formula A

Lipids of Formula A or pharmaceutically acceptable salts thereof can be prepared using methods that are well known to those of skill in the art. Methods that include fewer steps and/or are more economical than known syntheses are described below. Without intending to be limiting, such methods can be employed for the synthesis of representative, but non-limiting, compounds 5-45 of Table 1. Those skilled in the art would appreciate that alternative starting materials could be employed in the same sequences, leading to congeners of compound 5-44 as defined by Formula A. This includes, without limitation, congeners of compounds 5-44 incorporating moieties of Formula C, and/or N-alkylglycines other than sarcosine, and/or aminoacids such as beta-alanine and its N-alkyl congeners, wherein the amino group is at a position other than the carbonyl alpha-position, and/or enantioenriched or racemic cyclic aminoacids other than proline, such as azetidine 2-carboxylic acid, pipecolinic acid, and the like, and other enantioenriched or racemic, naturally occurring or non-naturally occurring aminoacids. Therefore, the schemes set forth below are merely illustrative of select embodiments.

Lipids of Formula A wherein and A¹ and A² are O and A³ is C can be prepared from ketones having the general structure of Formula E. One method for the synthesis of ketones of Formula

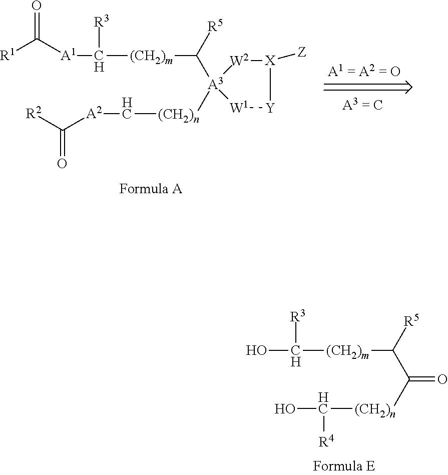

E wherein R³=R⁴ is exemplified, without intending to be limiting, with the preparation of 51-53 (Scheme 2). As described in co-owned and co-pending WO 2022/246555 (incorporated herein by reference), the Claisen

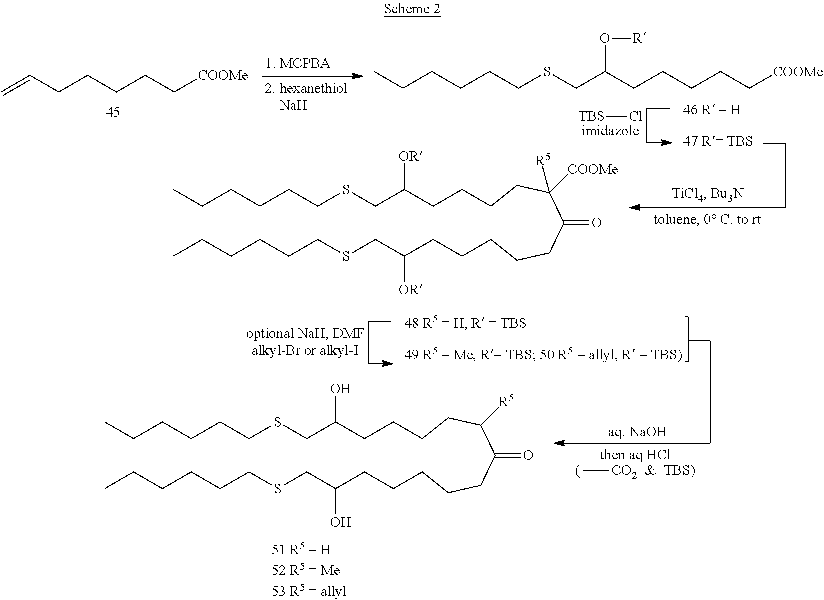

condensation of an ester such as 47 under Mukaiyama conditions produces 48, which may be optionally alkylated at the active methine position to give 49 or 50. Hydrolysis, decarboxylation, and desilylation of 48 and 49-50 transform them into 51 and 52-53, respectively.

As described in co-owned and co-pending U.S. provisional patent application No. 63/410,261 (incorporated herein by reference), another method for the synthesis of the foregoing ketones starts with the Mukaiyama-Claisen condensation of an ester such as 45. The resulting 54 can then be optionally alkylated, for example, methylated, at the active methine position to give 55 (Scheme 3). Hydrolysis and decarboxylation convert 54 and 55 into 56 and 57, respectively. The latter can be subjected to double bond epoxidation resulting in formation of 58 and 59, respectively, which upon oxirane cleavage with, for example, 1-hexanethiol, produce 51 and 52.

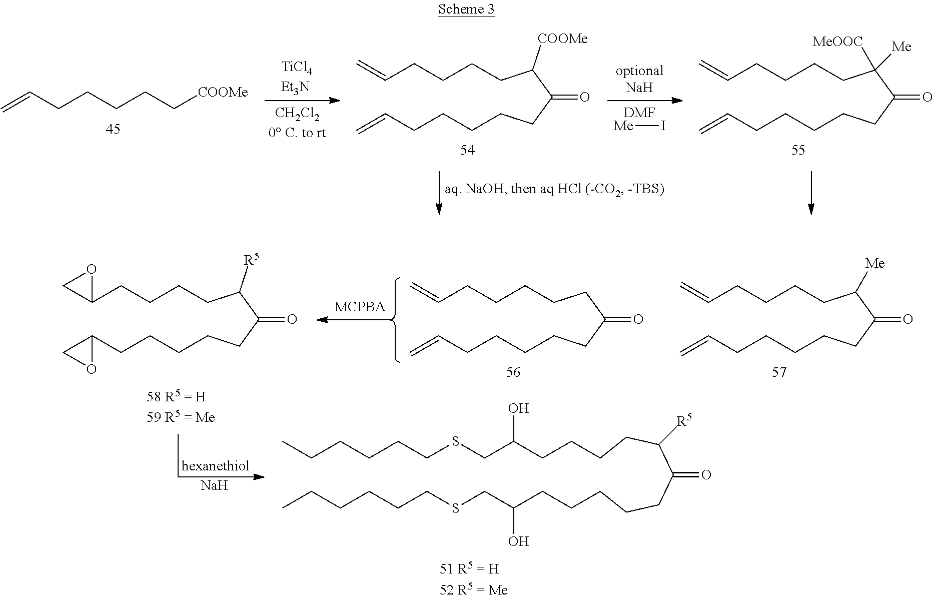

As described in co-owned and co-pending WO 2023/147657 (incorporated herein by reference), another method for the synthesis of ketones of Formula E wherein m=n and R³=R⁴=H starts with the Claisen condensation of a lactone under Mukaiyama conditions. Without intending to be limiting, this step is exemplified in Scheme 4 with the conversion of caprolactone, 60, into 61. In certain embodiments, the OH group in a compound such as 61 can be protected, for example as a silyl ether such as a tert-butyldimethylsilyl ether. The resulting 62 can then be alkylated at the reactive methine under basic conditions to produce substances such as 63-65. Lactone saponification and acidification of the reaction mixture to a pH of about 2 results in formation of ketoacids 66, which undergo decarboxylation to 67-69. The latter can be desilylated by treatment with a source of

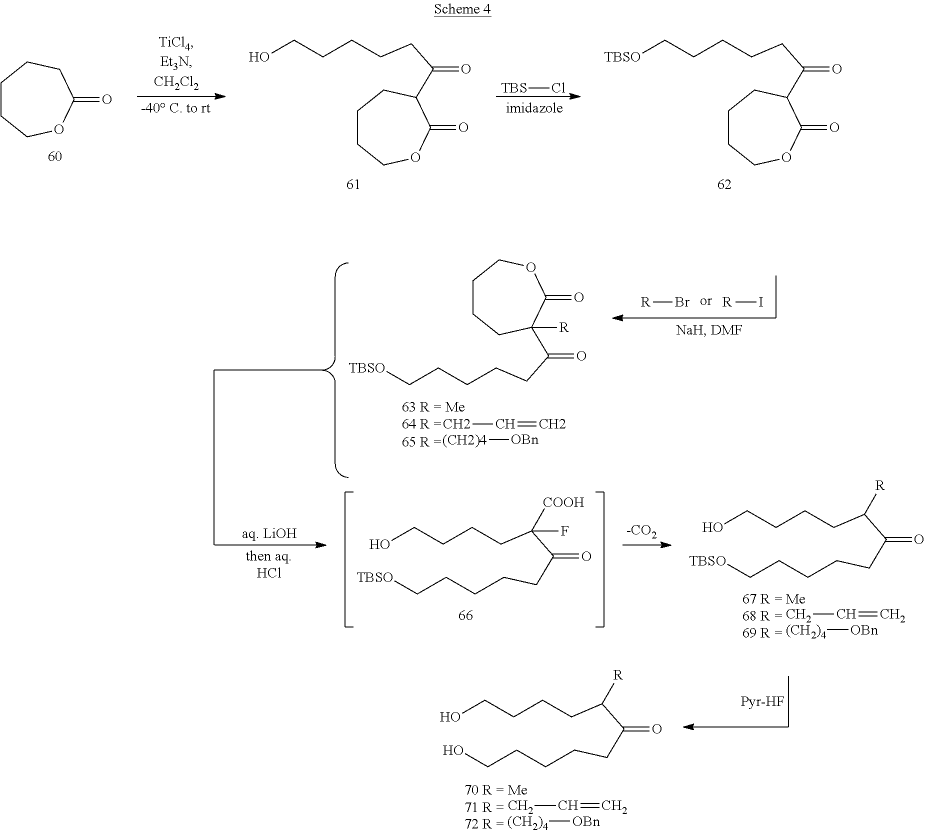

fluoride ion, for example, pyridine-HF complex, to give 70-72. Desilylation of compounds such as 67-69 can optionally be achieved by acidifying the saponification mixture to a pH of about 0 and allowing sufficient time for complete release of the silyl group.

Certain lipids of Formula A are best synthesized from a dihydroxyketone such as 74 (Scheme 5). In such cases, a product such as 61 can be advantageously converted directly into 74 by lactone hydrolysis and decarboxylation of the intermediate ketoacid 73. Furthermore, the steps of Claisen-Mukaiyama condensation, hydrolysis and decarboxylation can be most advantageously carried out in a “one-pot operation”, meaning that synthetic intermediates such as 61 and 73, while isolable, need not be isolated. The person skilled in the art will appreciate that alternative lactones can be

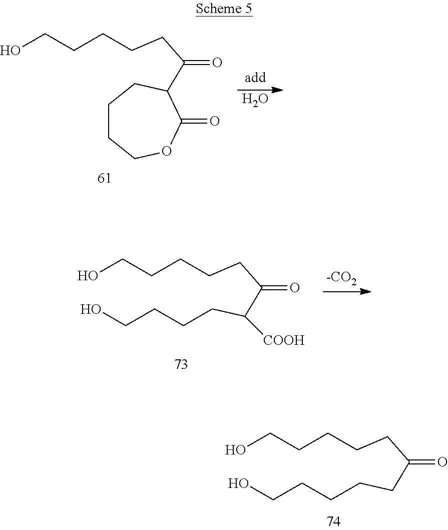

subjected to the conditions of Scheme 5 to produce congeners of dihydroxyketone 74. The reaction sequence in Scheme 5 is thus merely illustrative and should not be construed as limiting.

The synthesis of certain congeners of ketones 70-72 and 74 by the lactone method would require starting lactones that are uneconomical and/or not readily available. As described in co-owned and co-pending WO 2022/246555 and WO 2023/147657, in such cases the desired products can be manufactured from monoester derivatives of dicarboxylic acids. Without intending to be limiting, this is exemplified in Scheme 6 with the preparation of 79-80, the synthesis of which from a lactone would have to start from costly oxocan-2-one. In contrast, economical monomethyl azelate, 75, can be transformed into 76 by selective carboxy group reduction and protection of the OH. Claisen-Mukaiyama condensation converts 76 into 77, which can be optionally alkylated, for example, methylated, at the active methine to give 78. Compounds 77-78 can then be converted into 79-80, respectively, buy the method outlined above in Scheme 4.

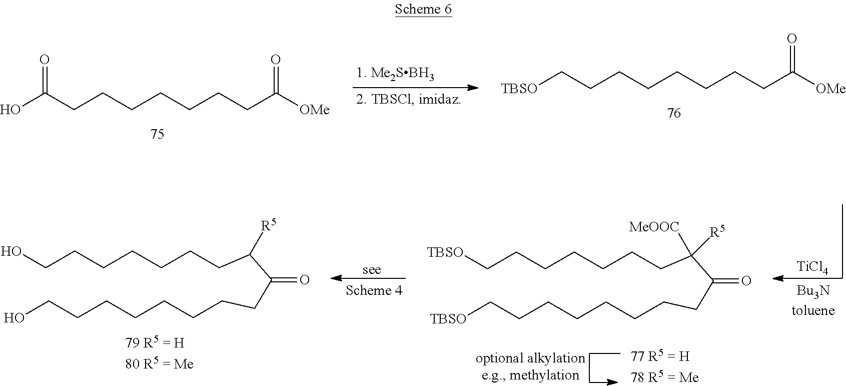

Yet another method for the synthesis of ketones of Formula E wherein R⁵=H is described in co-owned and co-pending U.S. provisional patent application No. 63/445,854. Accordingly, a reagent such as tosyl methyl isocyanide (TosMIC, 83), can doubly alkylated with an alkyl halide or sulfonate under basic conditions to produce a derivative that can be transformed into the desired ketone. Without intending to be limiting, this is exemplified in Scheme 7 with the synthesis of ketone 85 from ester 47 of Scheme 2. It is noted that the acid hydrolysis of 84 can be advantageously carried out under conditions that effect the concomitant release of the silyl protecting groups. The same method can be used to prepare ketones such as 56 of Scheme 3 by alkylation of TosMIC with 7-bromo-1-hepene, followed by acidic hydrolysis.

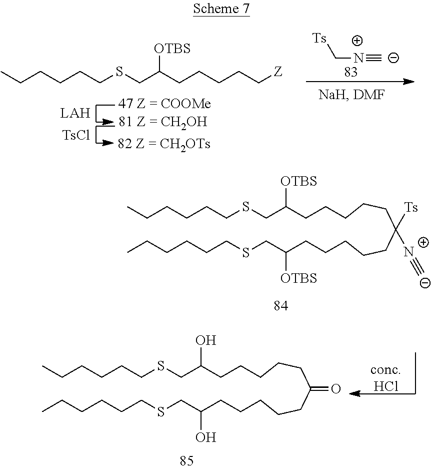

It is apparent that the synthetic diagram of Scheme 7 produces a ketone of Formula E wherein R³ and R⁴ are identical. However, the foregoing Provisional Application also teaches that a reagent such as TosMIC can be sequentially alkylated with two different alkyl halides or sulfonates. This leads to an ultimate ketone of Formula E wherein R³ and R⁴ are different. Without intending to be limiting, this is exemplified in Scheme 8 with the synthesis of ketone 89.

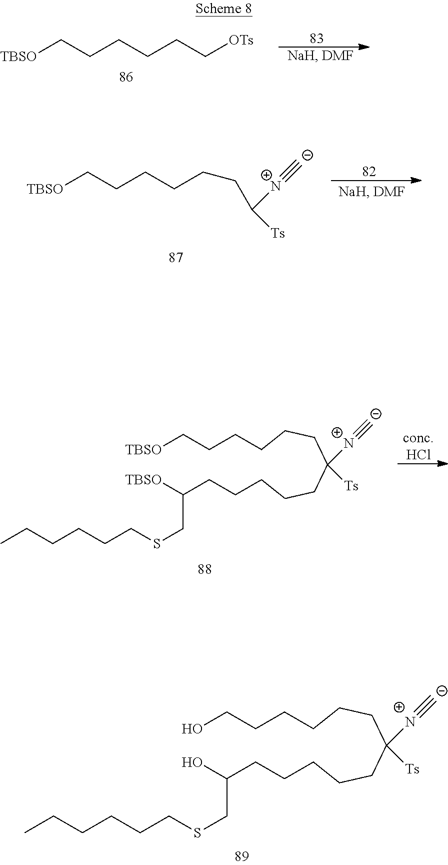

Lipids of Formula A wherein and A¹ and A² are O, A³ is N and R⁵ is (CH₂)_{q}—OH can be made from ketones having the general structure of Formula F, wherein PG is an oxygen protecting group such as a trialkylsilyl group; for example, a tert-butyldimethylsilyl group. Without intending to be limiting, the preparation of ketones 68-70 of Scheme 4 illustrates one method for the synthesis of ketones of Formula F. Without intending to be limiting, another method entails the selective silylation of the primary OH group in a ketone such as 89 of Scheme 8.

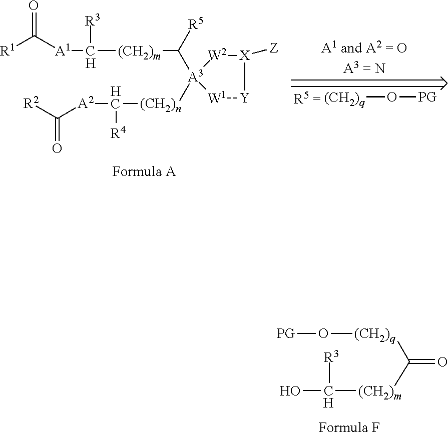

Lipids of Formula A wherein A³ is C, A¹ and A² are absent and R³ and R⁴ are both H can be prepared from ketodiacids having the general structure of Formula G. As described in co-owned and co-pending WO 2023/147657, ketodiacids of Formula G can be advantageously synthesized from a ketene dimer obtained by dehydrohalogenation of a half-ester/half

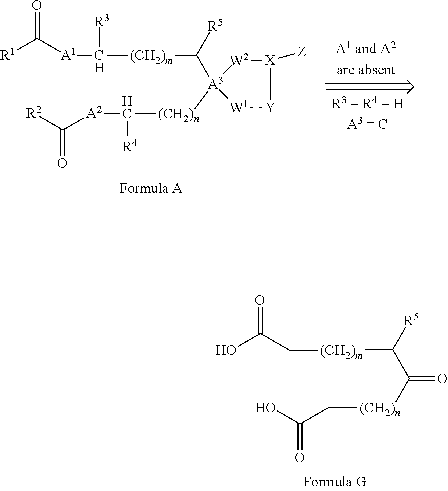

acid chloride derivative of a dicarboxylic acid monoester (Sauer, J. C. J. Am. Chem. Soc. 1947, 69, 2444; incorporated herein by reference). Without intending to be limiting, this is exemplified below with the preparation of compounds 94 and 97. The synthesis starts with the conversion of the acid chloride derivative 91 of monoethyl adipate, 90, into ketene dimer 92 (Scheme 9). The wavy bond in 92 signifies that the double bond can be of E- or Z-configuration.

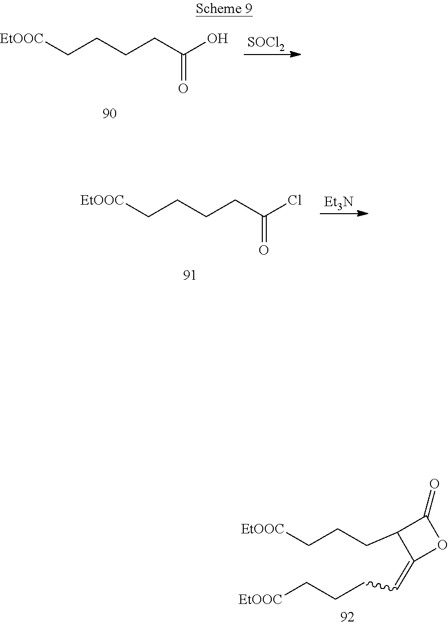

Compound 92 is isolable. However, in certain embodiments it is advantageous to convert 92 directly into a ketodiacid of Formula G wherein R5 is H. This can be achieved by treating 92 with aqueous alkali followed by aqueous acid, whereupon the intermediate beta-ketoacid 93 undergoes decarboxylation to produce 94 (Scheme 10). Alternatively, 92 can be treated with methanolic K2CO3, resulting in formation of beta-ketoester 95, which can be alkylated, for example, methylated, at the active methine to give derivative 96. Hydrolysis and decarboxylation transform the latter into 97, which is a ketodiacid of Formula G wherein R5 is alkyl.

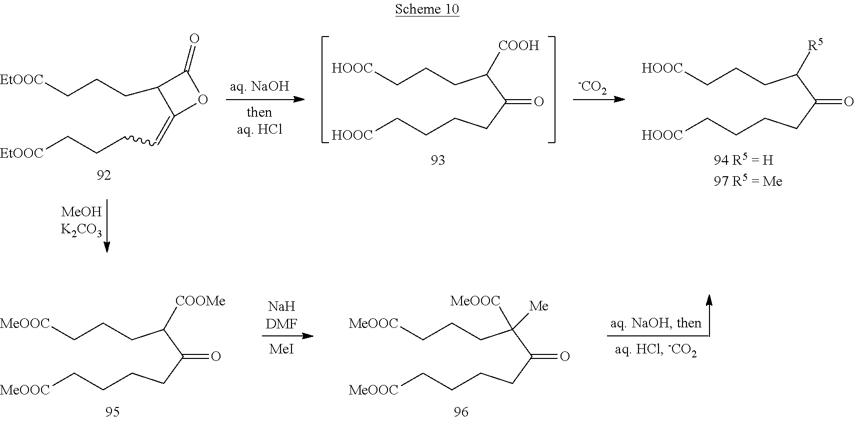

Lipids of Formula A wherein A² is N and R³ is H can be prepared using synthetic steps that are described in detail in co-pending and co-owned WO 2023/173203, which is incorporated herein by reference. As described in the foregoing disclosure, one such step entails reacting an appropriate aminoalcohol or an O-protected variant thereof, represented in Scheme 11 with the generic formula 98, wherein Z is H or an appropriate protecting group, with a suitable alkyl halide or sulfonate, resulting in formation of different products depending on the conditions. Specifically, a primary amine such as 98 can be doubly alkylated in a single step by heating in acetonitrile with an appropriate alkyl halide or sulfonate in the presence of a base, for example, Na₂CO₃, whereupon a double N-alkylation of the starting

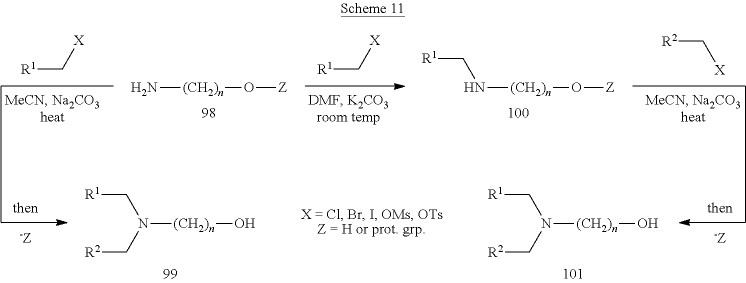

amine occurs. Release of the Z protecting group, if present, produces a compound of general structure 99. Alternatively, primary amine 98 can be mono-N-alkylated by treatment with an appropriate alkyl halide or sulfonate in DMF at room temperature in the presence of a base, for example, K₂CO₃. This results in formation of secondary amine 100, which can be N-alkylated a second time by heating in acetonitrile with a different alkyl halide or sulfonate in the presence of Na₂CO₃. Release of the Z protecting group, if present, produces 101.

The synthesis of some lipids of this Disclosure requires certain sulfur-containing acids, such as the representative, but nonlimiting, 102 and 103 (Scheme 12), methods for the preparation of which and their congeners are provided in co-pending and co-owned U.S. provisional patent application No. 63/410,281 (102) and PCT application No. PCT/CA2023/050644 (102), both incorporated herein by reference.

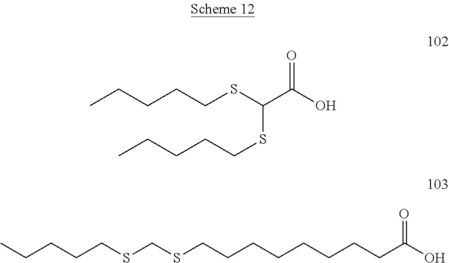 [SMILES: CCCCCSC(SCCCCC)C(=O)O ; CCCCCSCSCCCCCCCCC(=O)O]

The preparation of certain lipids in Table 1 requires aminoacid-derived compounds 104-110 (Scheme 13). Compounds such as 104-110 can be made by N-acylation of an aminoacid ester or a corresponding salt, such as a hydrochloride, with a carboxylic acid in the presence of a coupling agent, optionally in the presence of 4-dimethylaminopyridine (DMAP), or with an acid chloride in the presence of an appropriate base, followed by saponification of the ester and acidic workup.

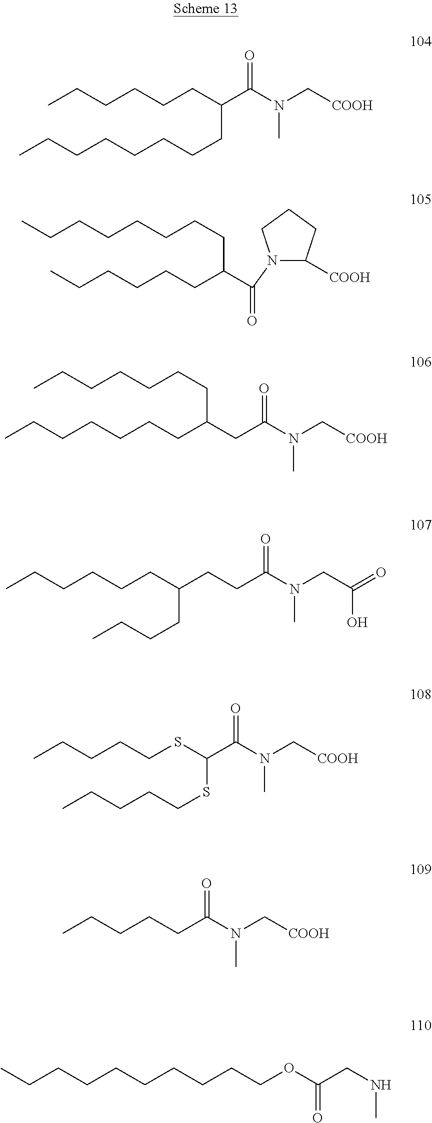

Without intending to be limiting, this is exemplified in Scheme 14 with the synthesis of 104, in which case the aminoacid ester is sarcosine methyl ester hydrochloride, 111, the acid is 2-hexyldecanoic acid, the coupling agent is a carbodiimide such as EDCI. Compounds 105-109 can be made in a like fashion from the appropriate aminoacid- and carboxylic acid educts.

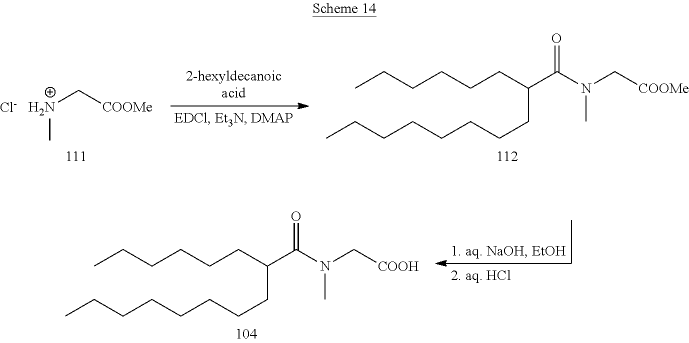

Compounds such as 110 can be prepared from an N-protected aminoacid by esterification with an alcohol in the presence of a coupling agent and optionally DMAP, followed by release of the N-protecting group. Without intending to be limiting, this is exemplified in Scheme 15 with the synthesis of 110, in which case the N-protected aminoacid is N-BOC sarcosine, 113, the alcohol is decanol, the coupling agent is a carbodiimide such as EDCI, and the release of the N-BOC group is achieved with HCl in dioxane.

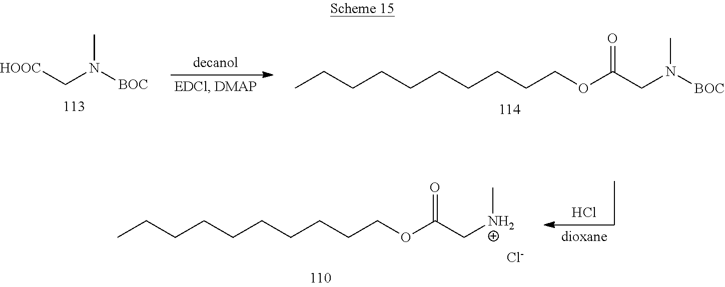

Certain steps of the synthesis of the lipids of this Disclosure and their congeners entail subjecting a dihydroxyketone of Formula E to esterification of the OH groups with appropriate carboxylic acids. As described in co-owned and co-pending WO 2023/147657, the esterification reaction can be carried out under conditions that result in the formation of a diester product comprising two identical acyl groups (i.e., a symmetrical diester), or a monoester. The monoester can subsequently be transformed into a diester comprising two different acyl groups (i.e., an unsymmetrical diester) by esterification of the remaining OH group with a different acid. Without intending to be limiting, this is exemplified in Scheme 16 by the conversion of 74 into symmetrical diester 115, by reaction with at least two molar equivalents of acid 104 in the presence of a condensing agent, for example, T3P or a carbodiimide such as EDCI, optionally in the presence of DMAP, or into monoester 118, by reaction with one molar equivalent or less of 104 in the presence of a condensing agent such as a carbodiimide, for example, EDCI, and optionally DMAP. Compound 118 can subsequently be transformed into unsymmetrical diester 119 by reaction with at least one molar equivalents of 2-hexyldecanoic acid in the presence of a condensing agent such as a carbodiimide, for example, EDCI, and optionally DMAP. Compounds 115 and 117 are the precursors of several lipids of Table 1. The person skilled in the art will appreciate that congeners of compounds 115 and 117 can be prepared by the same method through the union of an appropriate dihydroxyketone of Formula E with suitable carboxylic acids.

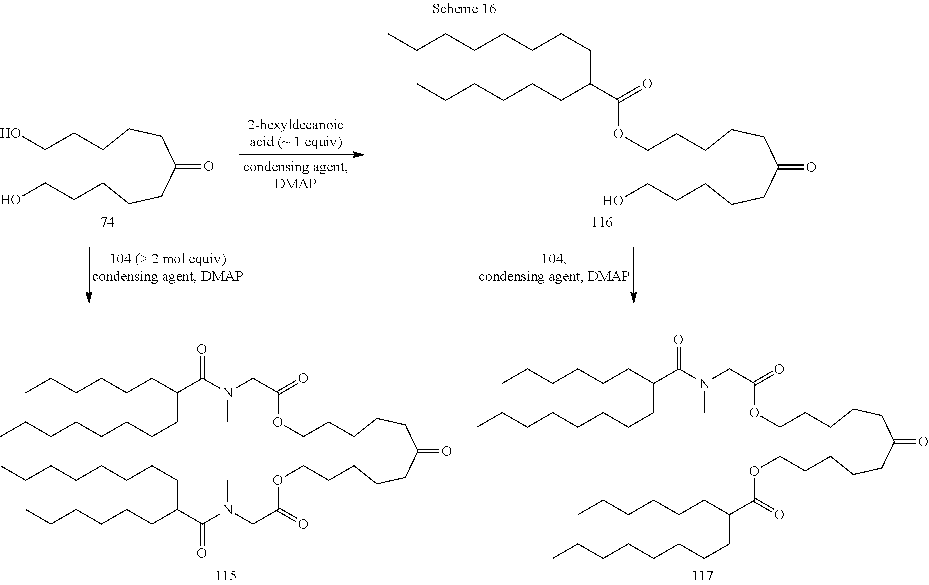

Certain steps of the synthesis of the lipids of this Disclosure and their congeners entail subjecting a ketone having the structure of Formula F to protection of the OH group, for example as a silyl ether. This is exemplified, without intending to be limiting, with the conversion of compound 116 above into tert-butyldiphenylsilyl ether 118, which is the precursors of lipid 36 (Scheme 17).

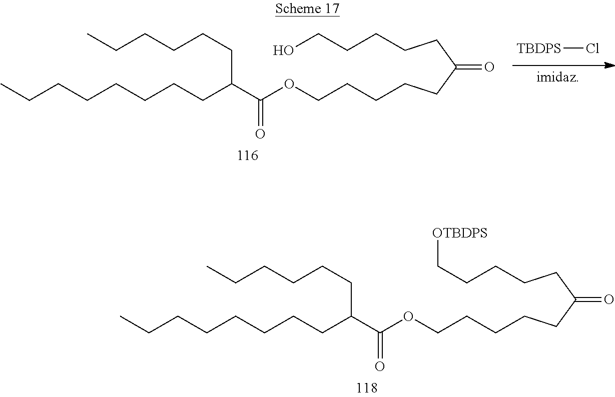

Certain steps of the synthesis of the lipids of this Disclosure and their congeners involve transforming a ketodiacid of Formula G into ester or amide derivatives. As described in co-owned and co-pending WO 2023/147657, these reactions can be carried out under conditions that result in the formation of a symmetrical diester or diamide product (i.e., one in which both COOH groups have combined with the same alcohol or amine) or a monoester or monamide product. The monoester or monoamide can subsequently be transformed into an unsymmetrical diester (i.e., one in which the two COOH groups have combined with two different alcohol), an unsymmetrical diamide (i.e., one in which the two COOH groups have combined with two different amines), or an amidoester (i.e., a product in which one COOH group has combined with an alcohol and the other has combined with an amine). Without intending to be limiting, this is exemplified in Scheme 18 by the conversion of 94 into ester 119 by reaction with one molar equivalents of 2-hexyldecan-1-ol in the presence of a condensing agent, for example, a carbodiimide such as EDCI, optionally in the presence of DMAP, and the transformation of 119

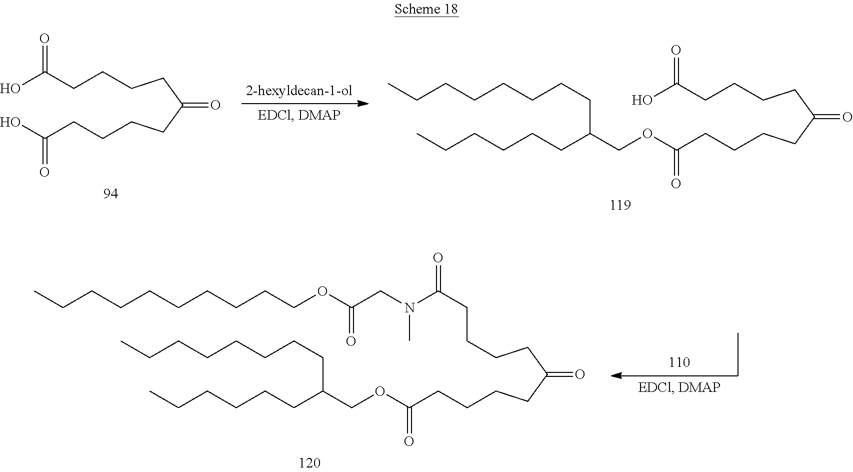 [SMILES: O=C(O)CCCCC(=O)CCCCC(=O)O ; CCCCCCCCC(CCCCCC)COC(=O)CCCCC(=O)CCCCC(=O)O ; CCCCCCCCCCOC(=O)CN(C)C(=O)CCCCC(=O)CCCCC(=O)OCC(CCCCCC)CCCCCCCC]

into 120 by reaction with one molar equivalents of 110 in the presence of a condensing agent, for example, a carbodiimide such as EDCI, optionally in the presence of DMAP. Compound 120 is the precursors of lipid 11 of Table 1. The person skilled in the art will appreciate that congeners of compounds 120 can be prepared by the same method through the union of an appropriate ketodiacid of Formula G with suitable alcohols or amines.

The ketone group in representative, but nonlimiting, compounds 115, 117, 118, 120, and their congeners can be transformed into any ionizable head group of type 1-9 by methods that are well known to those skilled in the art, thus achieving conversion into appropriate lipids. Representative examples, which are by no means to be construed as limiting, are provided below.

The synthesis of lipid 5 requires the introduction of a type 1 ionizable head group on ketone 115 of Scheme 16. Accordingly, the ketone in 115 is selectively reduced to alcohol 121 with a hydride reagent, for example, sodium borohydride, in an appropriate solvent, for example, an alcohol such as isopropanol. Alcohol 121 is then esterified with 4-(dimethylamino)-butanoic acid, 122, or its hydrochloride salt, in the presence of a condensing agent, for example, a carbodiimide such as EDCI, and optionally DMAP (Scheme 19).

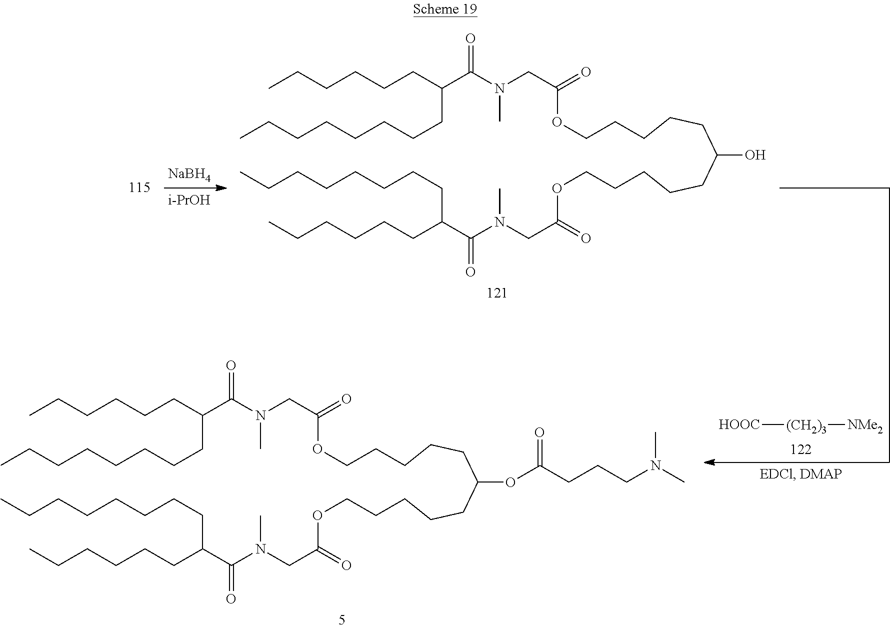

Lipids 6-13 can be made by the same method starting from corresponding ketones, as shown in Schemes 20-22 below:

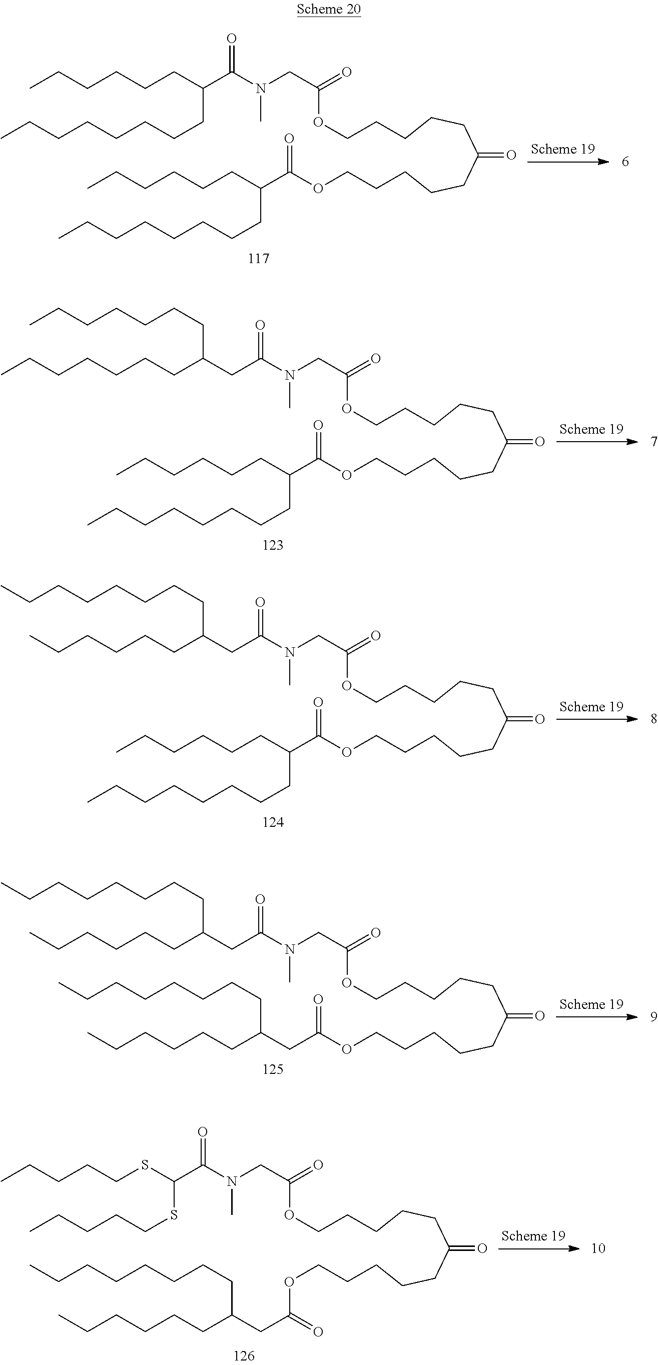 [SMILES: CCCCCCCCC(CCCCCC)C(=O)OCCCCCC(=O)CCCCCOC(=O)CN(C)C(=O)C(CCCCCC)CCCCCCCC ; CCCCCCCCC(CCCCCC)C(=O)OCCCCCC(=O)CCCCCOC(=O)CN(C)C(=O)CC(CCCCCCC)CCCCCCC ; CCCCCCCCC(CCCCCC)CC(=O)N(C)CC(=O)OCCCCCC(=O)CCCCCOC(=O)C(CCCCCC)CCCCCCCC ; CCCCCCCCC(CCCCCC)CC(=O)OCCCCCC(=O)CCCCCOC(=O)CN(C)C(=O)CC(CCCCCC)CCCCCCCC ; CCCCCCCCC(CCCCCC)CC(=O)OCCCCCC(=O)CCCCCOC(=O)CN(C)C(=O)C(SCCCCC)SCCCCC]

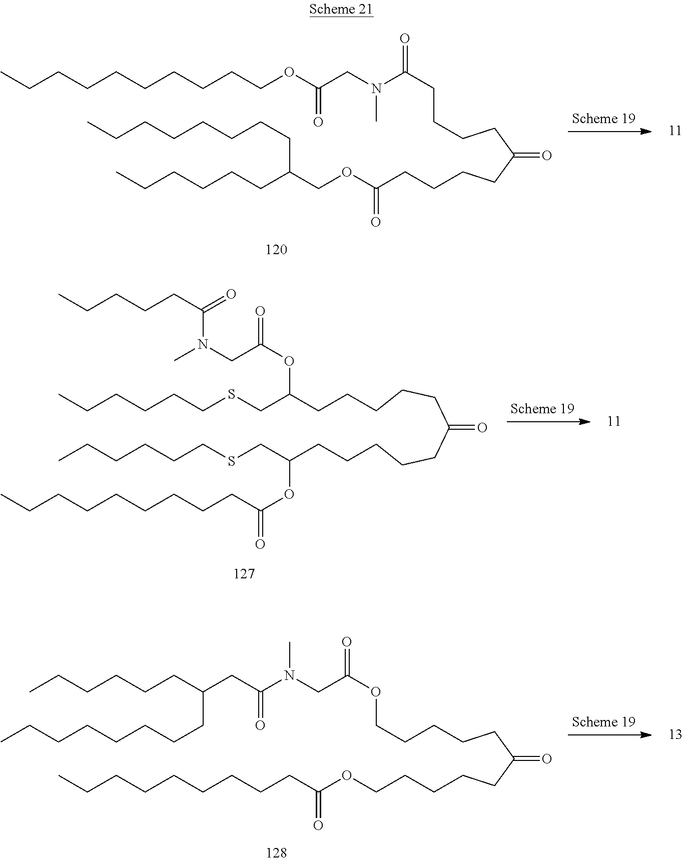 [SMILES: CCCCCCCCCCOC(=O)CN(C)C(=O)CCCCC(=O)CCCCC(=O)OCC(CCCCCC)CCCCCCCC ; CCCCCCCCCC(=O)OC(CCCCCC(=O)CCCCCC(CSCCCCCC)OC(=O)CN(C)C(=O)CCCCC)CSCCCCCC ; CCCCCCCCCC(=O)OCCCCCC(=O)CCCCCOC(=O)CN(C)C(=O)CC(CCCCCC)CCCCCCCC]

The synthesis of lipid 14 requires the introduction of a type 2 ionizable head group on ketone 115 of Scheme 16. Accordingly, the ketone is ketalized with 1,2,4-butanetriol in the presence of a catalyst such as PPTS, in an appropriate solvent, for example, toluene, at a suitable temperature, for example, at reflux, with continuous removal of the water produced during the reaction, for example, by the use of a Dean-Stark trap, to produce ketal 129. The OH group in 129 is transformed into a leaving group, for example by reaction with a sulfonyl chloride, e.g., p-toluenesulfonyl chloride, and the resulting tosylate 130 is reacted with dimethylamine in an appropriate solvent at a suitable temperature, for example in THF, optionally under microwave irradiation, to produce 14 (Scheme 22).

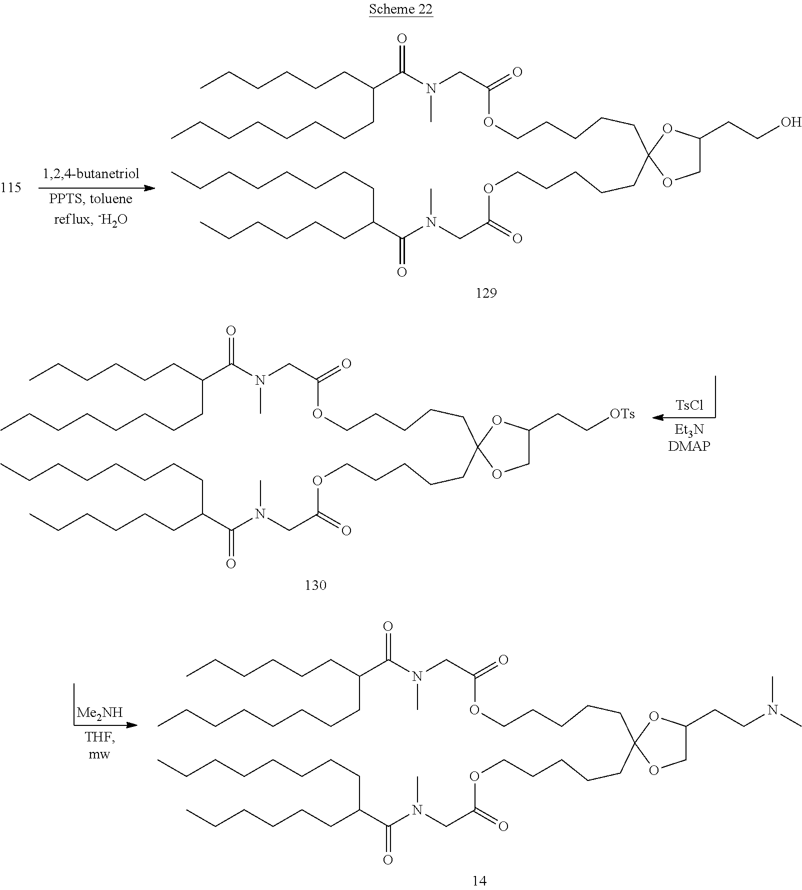

Lipids 15 and 16 can be made by the same method from ketones 123 and 117 of Scheme 20 (Scheme 23).

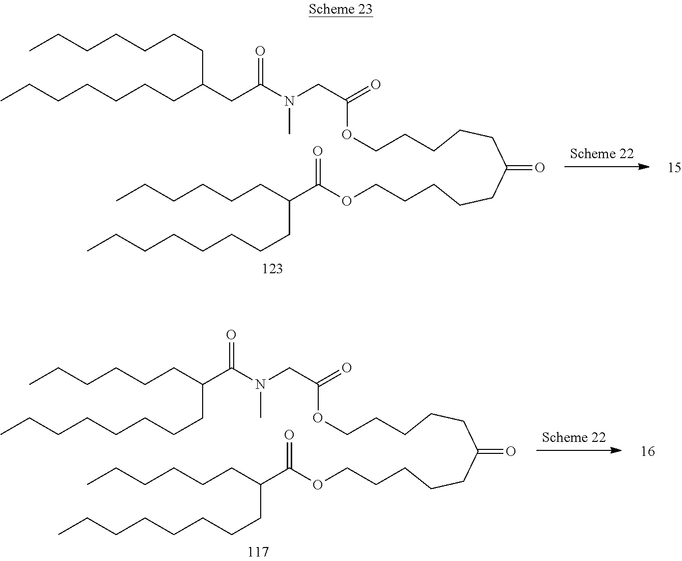 [SMILES: CCCCCCCCC(CCCCCC)C(=O)OCCCCCC(=O)CCCCCOC(=O)CN(C)C(=O)CC(CCCCCCC)CCCCCCC ; CCCCCCCCC(CCCCCC)C(=O)OCCCCCC(=O)CCCCCOC(=O)CN(C)C(=O)C(CCCCCC)CCCCCCCC]

The synthesis of lipid 17 requires the introduction of a type 3 ionizable head on ketone 115 of Scheme 16. This can be done by esterification of the OH group in ketal 129 of Scheme 22 with 4-(dimethylamino)-butanoic acid or its hydrochloride salt, in the presence of a condensing agent, for example, a carbodiimide such as EDCI and optionally DMAP, as shown in Scheme 24.

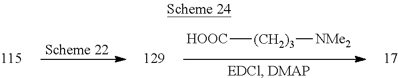

Lipid 18 can be prepared in an analogous manner from ketone 117 of Scheme 20 (Scheme 25).

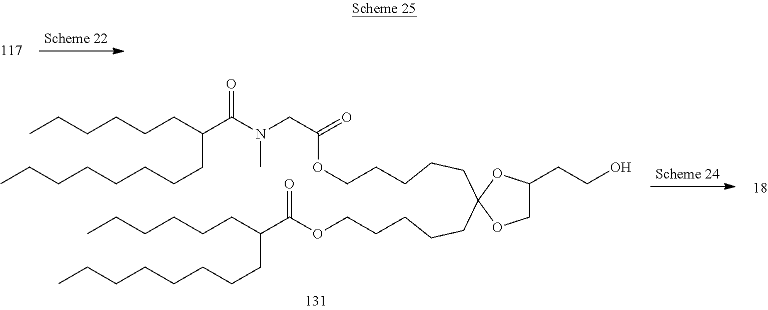 [SMILES: "Scheme 22" ; CCCCCCCCC(CCCCCC)C(=O)OCCCCCC1(CCCCCOC(=O)CN(C)C(=O)C(CCCCCC)CCCCCCCC)OCC(CCO)O1 ; "Scheme 24"]

The synthesis of lipid 19 requires the introduction of a type 9 ionizable head group on ketone 115 of Scheme 16. This can be done by glutaroylation of the OH group in alcohol 121 of Scheme 19, followed esterification of the resulting 132 with 3-(dimethylamino)-1-propanol in the presence of, for example, a carbodiimide such as EDCI and optionally DMAP (Scheme 26).

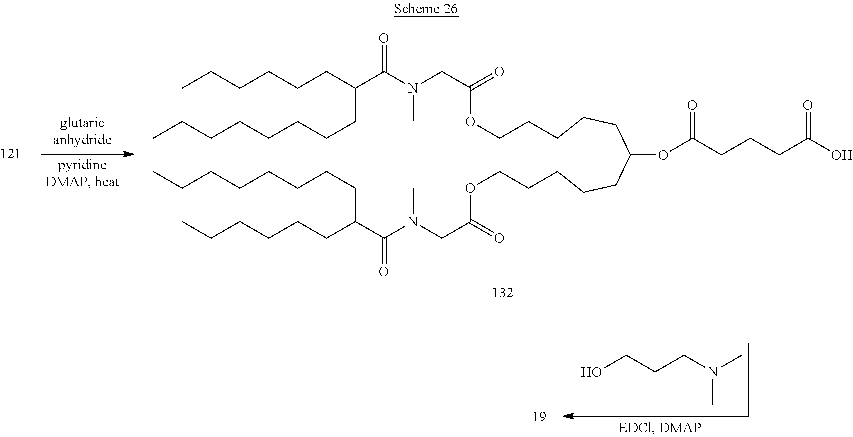

The synthesis of lipid 20 (Scheme 27) requires the introduction of a type 8 ionizable head group on ketone 115 of Scheme 16. Accordingly, ketone 115 can be reductively aminated with N^{I},N^{I}-dimethyl-propane-1,3-diamine in the presence of a suitable reducing agent, for example, sodium triacetoxyborohydride, and an acid catalyst, for example, acetic acid, to give 133. The secondary amino group in 133 is then reductively methylated by reaction with formaldehyde in the presence of a suitable reducing agent, for example, sodium triacetoxyborohydride, optionally in the presence of an acid catalyst, for example, acetic acid, to produce 20. Lipid 21 can be prepared by the same method starting from ketone 117 of Scheme 20.

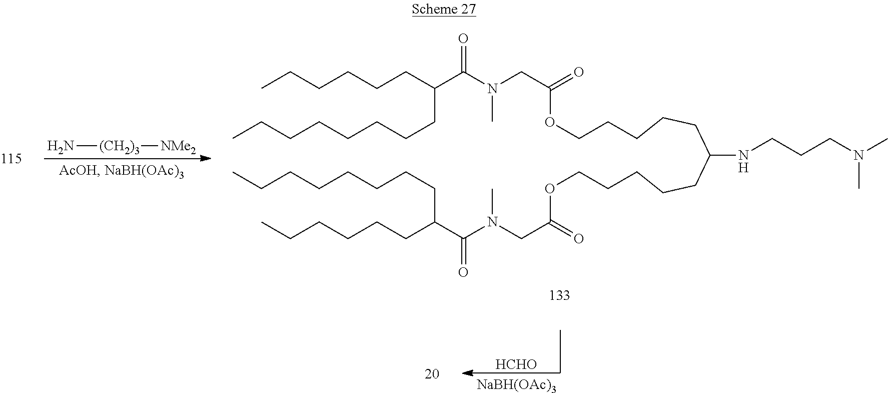

The synthesis of lipid 22 (Scheme 28) requires the introduction of a type 7 ionizable head group on ketone 117 of Scheme 20. Accordingly, reductive amination of 117 with an O-protected

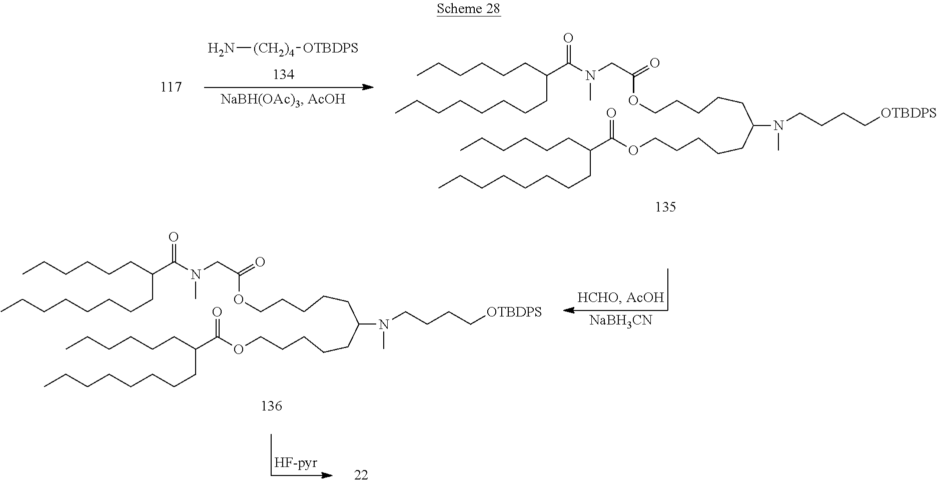

derivative of an aminoalcohol, for example, the O-TBDPS derivative 134 of 4-amino-1-butanol, forms secondary amine 135, which is reductively methylated to give compound 136. Release of the silyl group from 137 by reaction with a source of fluoride ion, for example, pyridine-HF complex, produces 22.

Lipids 23-35 can be prepared by the same method starting from appropriate ketones, O-protected amines, and aldehydes, as shown in Schemes 29-30.

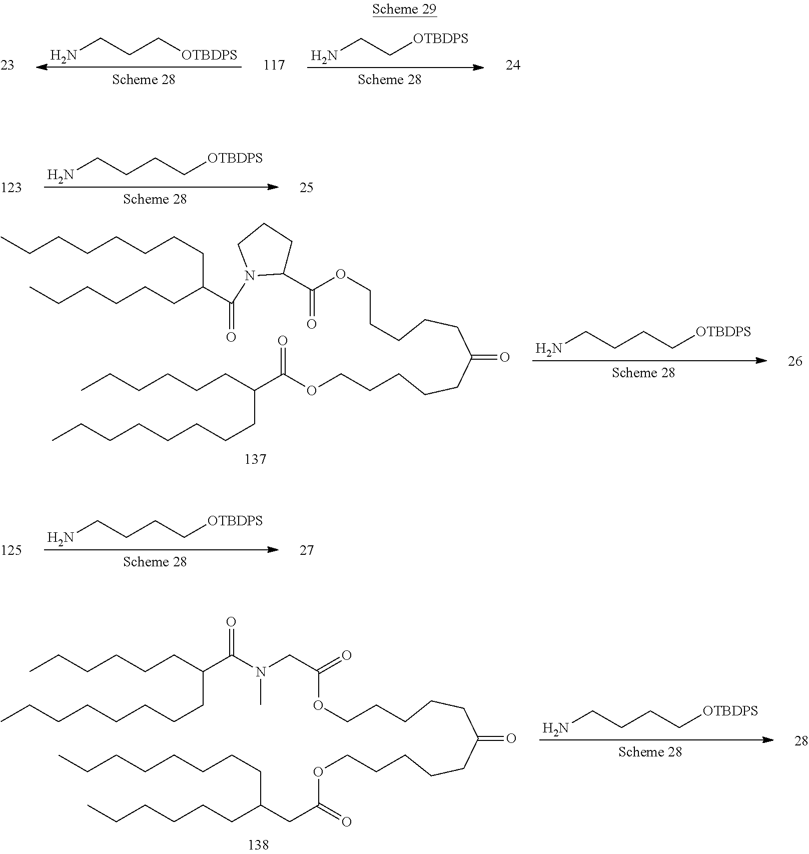

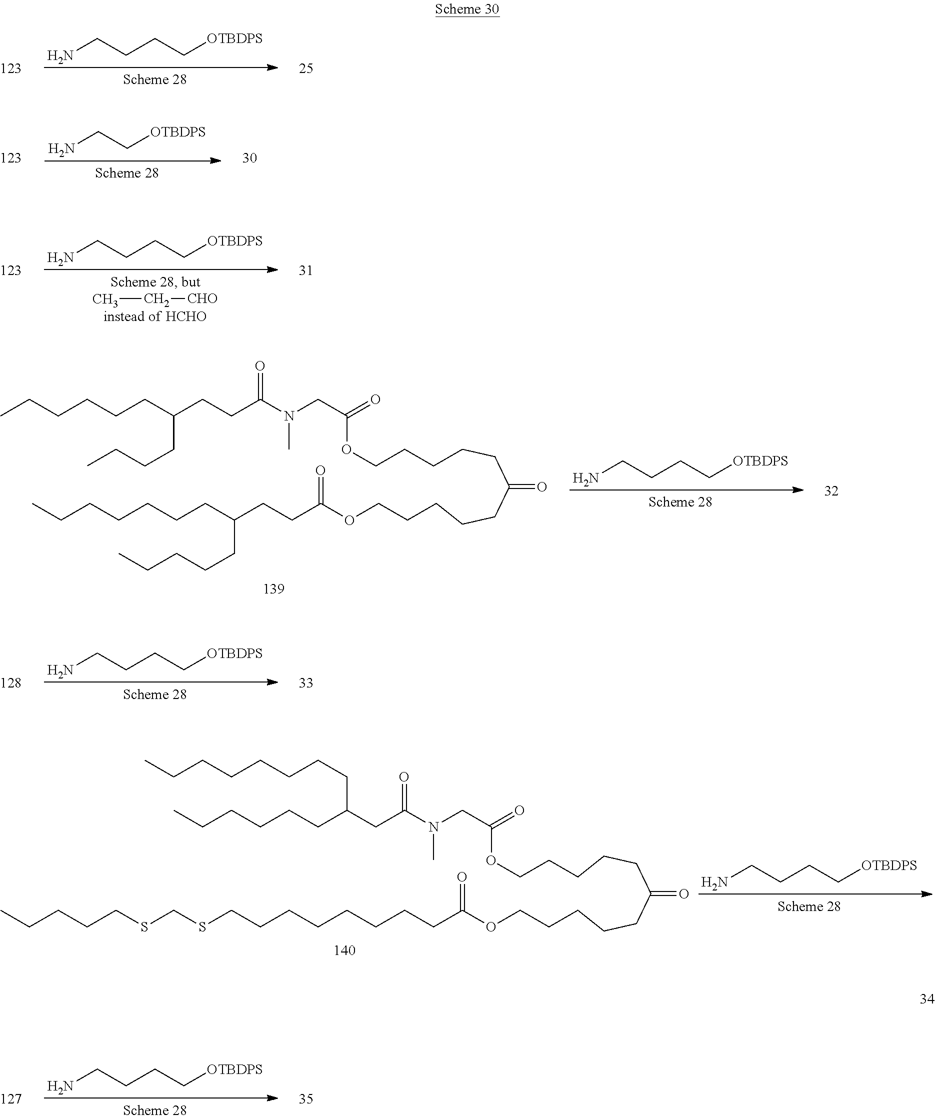

The synthesis of lipids 36-38 involves the reductive amination of a ketone of Formula F with an appropriate amino compound, such as 143 or 145 (Scheme 31), advantageously employed as hydrochloride salts. Without intending to be limiting, a method for producing such hydrochloride

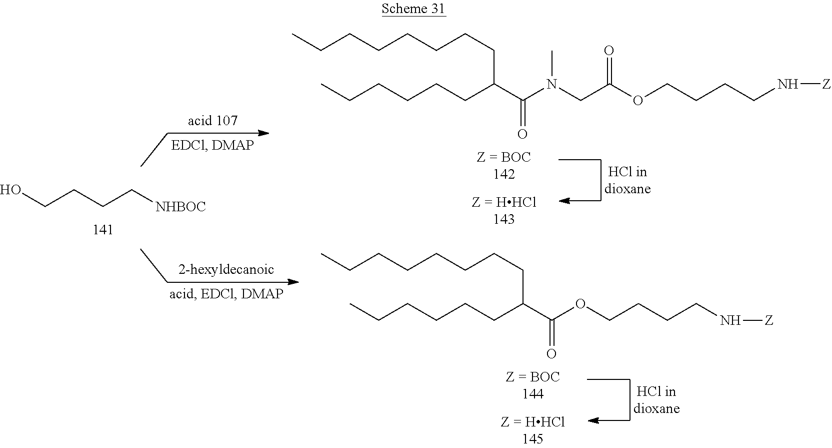

salts is shown in Scheme 31. The synthesis of lipid 36 from 118 and 143 is shown in Scheme 32.

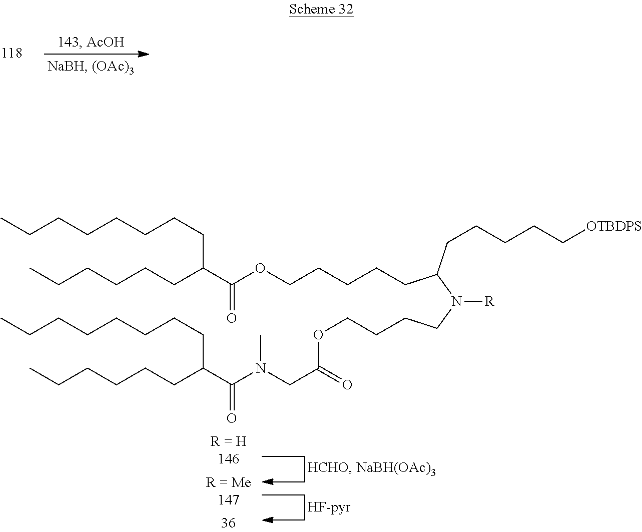

Lipid 37 can be made in a like manner from ketone 148 and hydrochloride 145 (Scheme 33).

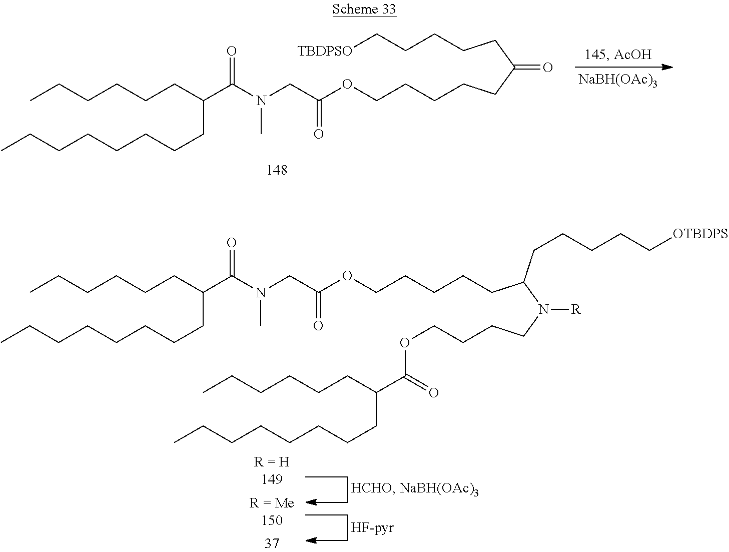

Lipid 38 can be made in a like manner from ketone 151 and 143 (Scheme 34).

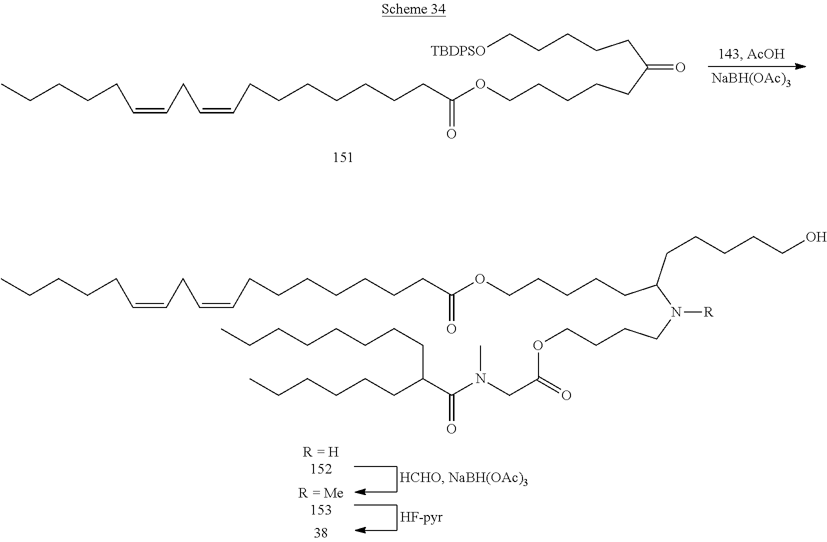

Lipids 39-44 can be synthesized according to the methods of Scheme 11 above. This requires alkyl halides 154-157 (Scheme 35). These compounds can be prepared by procedures that are well known to the person skilled in the art. For example, they can be made by esterification of an appropriate acid with 5-chloro-1-pentanol or 6-bromo-1-hexanol in the presence of a coupling agent, for example, a carbodiimide such EDCI and optionally DMAP.

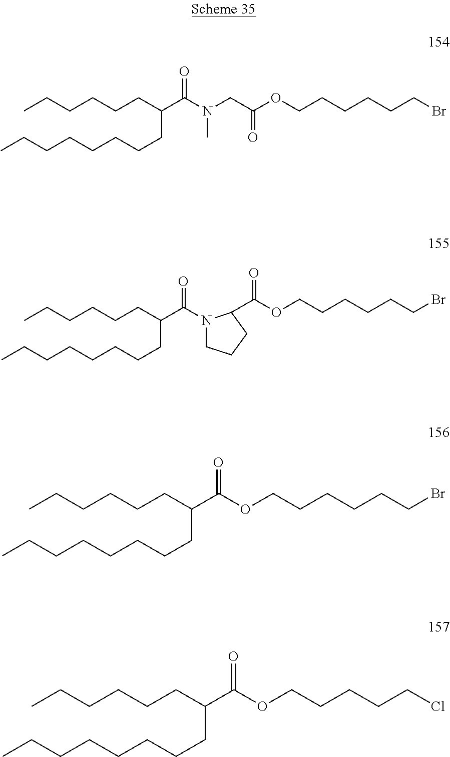 [SMILES: CCCCCCCCC(CCCCCC)C(=O)N(C)CC(=O)OCCCCCCBr ; CCCCCCCCC(CCCCCC)C(=O)N1CCCC1C(=O)OCCCCCCBr ; CCCCCCCCC(CCCCCC)C(=O)OCCCCCCBr ; CCCCCCCCC(CCCCCC)C(=O)OCCCCCCl]

The synthesis of lipid 39 entails the double N-alkylation of 4-amino-1-butanol with bromide 154 by heating in acetonitrile in the presence of Na₂CO₃ or K₂CO₃, optionally with microwave irradiation (Scheme 36).

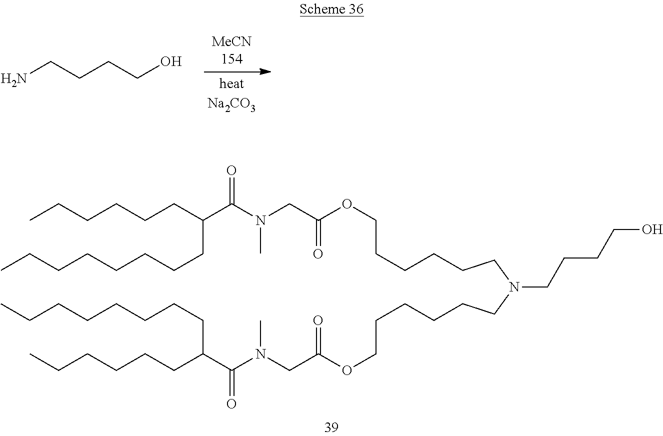 [SMILES: NCCCCO ; CCCCCCCCC(CCCCCC)C(=O)N(C)CC(=O)OCCCCCCN(CCCCO)CCCCCCOC(=O)CN(C)C(=O)C(CCCCCC)CCCCCCCC]

Lipid 40 can be prepared in a like manner from 4-amino-1-butanol and bromide 155 (Scheme 37).

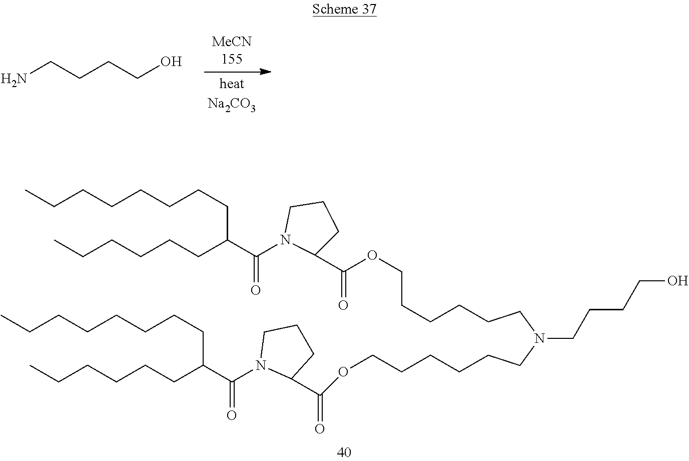 [SMILES: NCCCCO ; CCCCCCCCC(CCCCCC)C(=O)N1CCCC1C(=O)OCCCCCCN(CCCCO)CCCCCCOC(=O)C1CCCN1C(=O)C(CCCCCC)CCCCCCCC]

Lipid 41 can be synthesized by sequential N-alkylation of 4-amino-1-butanol with bromides 154 and 156 (Scheme 38).

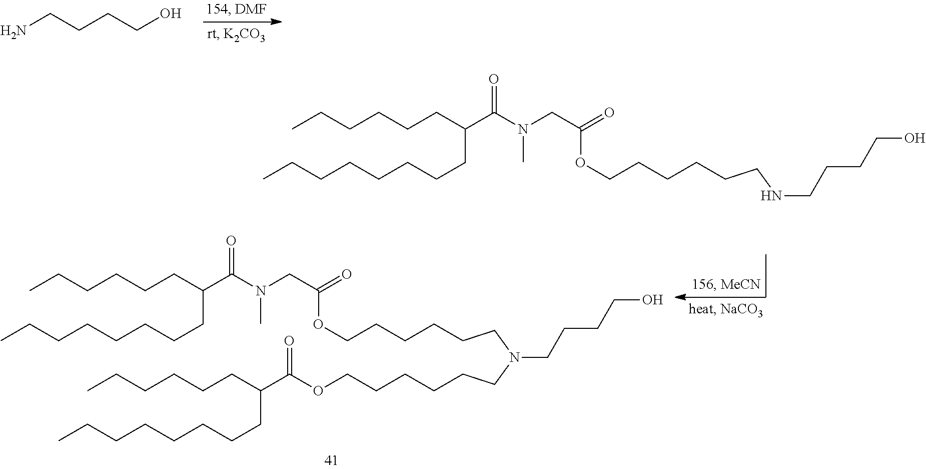 [SMILES: NCCCCO ; CCCCCCCCC(CCCCCC)C(=O)N(C)CC(=O)OCCCCCCNCCCCO ; CCCCCCCCC(CCCCCC)C(=O)OCCCCCCN(CCCCO)CCCCCCOC(=O)CN(C)C(=O)C(CCCCCC)CCCCCCCC]

Lipid 42 can be synthesized by sequential N-alkylation of 4-amino-1-butanol with bromides 155 and 156 (Scheme 39).

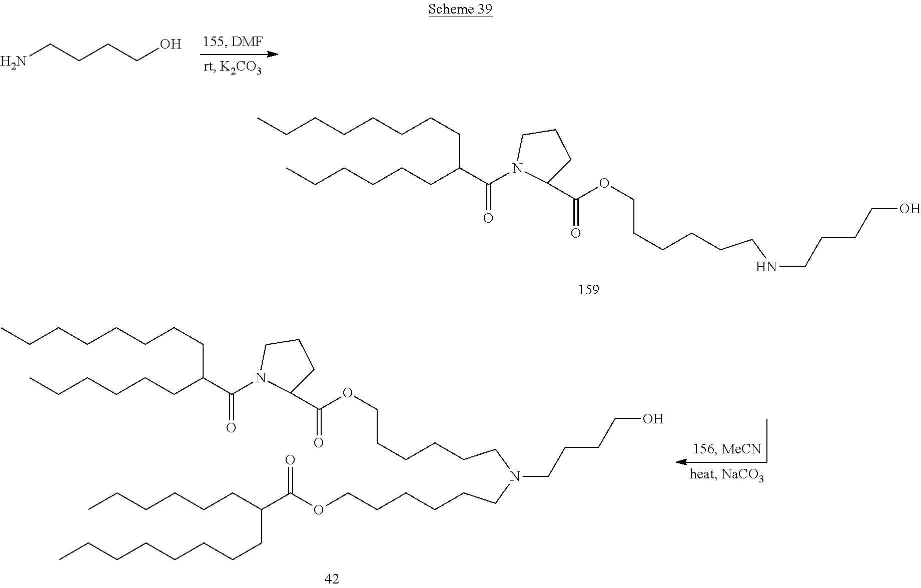 [SMILES: NCCCCO ; CCCCCCCCC(CCCCCC)C(=O)N1CCCC1C(=O)OCCCCCCNCCCCO ; CCCCCCCCC(CCCCCC)C(=O)OCCCCCCN(CCCCO)CCCCCCOC(=O)C1CCCN1C(=O)C(CCCCCC)CCCCCCCC]

Lipid 43 can be synthesized by N-alkylation of compound 159 with chloride 157 (Scheme 40).

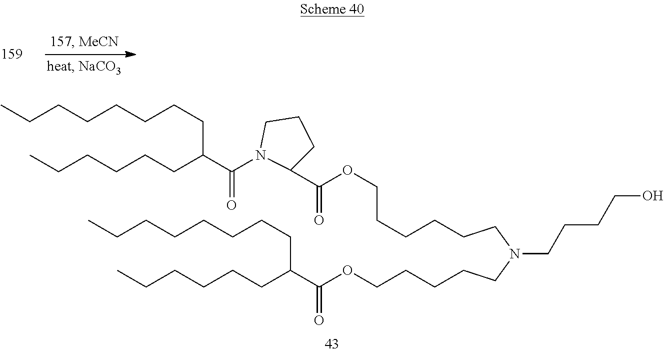 [SMILES: CCCCCCCCC(CCCCCC)C(=O)OCCCCCN(CCCCO)CCCCCCOC(=O)C1CCCN1C(=O)C(CCCCCC)CCCCCCCC]

Lipid 44 can be synthesized by N-alkylation of 158 with chloride 157 (Scheme 41).

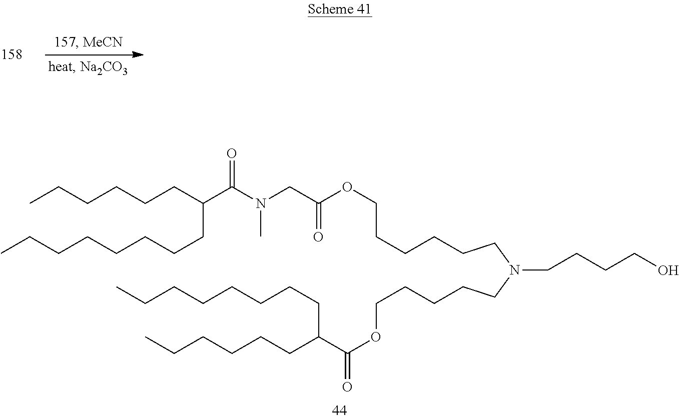 [SMILES: CCCCCCCCC(CCCCCC)C(=O)OCCCCCN(CCCCO)CCCCCCOC(=O)CN(C)C(=O)C(CCCCCC)CCCCCCCC]

### Formulation of the Lipid in a Delivery Vehicle

The lipids of the disclosure may be formulated in a variety of drug delivery vehicles (also referred to herein as a “delivery vehicle”) known to those of ordinary skill in the art. An example of a delivery vehicle is a lipid nanoparticle, which includes liposomes, lipoplexes, polymer nanoparticles comprising lipids, polymer-based nanoparticles, emulsions, and micelles.

In one embodiment, a lipid having the structure of Formula A of the disclosure is formulated in a delivery vehicle by mixing them with additional lipids, including helper lipids, such as vesicle forming lipids and optionally an aggregation inhibiting lipid, such as a hydrophilic polymer-lipid conjugate (e.g., PEG-lipid).

As set forth previously, a helper lipid includes a sterol, a diacylglycerol, a ceramide or derivatives thereof.

Examples of sterols include cholesterol, or a cholesterol derivative, such as cholestanol, cholestanone, cholestenone, coprostanol, cholesteryl-2′-hydroxyethyl ether, cholesteryl-4′-hydroxybutyl ether, beta-sitosterol, fucosterol, and the like.

Examples of diacylglycerols include dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof. In certain embodiments, the phospholipid is DPPC, DSPC, a DSPC-cholesterol conjugate or mixtures thereof. These lipids may be synthesized or obtained from natural sources, such as from egg. The DSPC-cholesterol conjugate is a lipid in which one of the acyl chains is substituted with a cholesterol moiety link to the head group by a succinate linker.

A suitable ceramide derivative is egg sphingomyelin or dihydrosphingomyelin.

Delivery vehicles incorporating the lipids of the disclosure can be prepared using a wide variety of well described formulation methodologies known to those of skill in the art, including but not limited to extrusion, ethanol injection and in-line mixing. In one embodiment, the preparation method is an in-line mixing technique in which aqueous and organic solutions are mixed using a rapid-mixing device as described in Kulkarni et al., 2018, ACS Nano, 12:4787 and Kulkarni et al., 2017, Nanoscale, 36:133347, each of which is incorporated herein by reference in its entirety.

The delivery vehicle can also be a nanoparticle that is a lipoplex that comprises a lipid core stabilized by a surfactant. Vesicle-forming lipids may be utilized as stabilizers. The lipid nanoparticle in another embodiment is a polymer-lipid hybrid system that comprises a polymer nanoparticle core surrounded by stabilizing lipid. Nanoparticles comprising lipids of the disclosure may alternatively be prepared from polymers without lipids. Such nanoparticles may comprise a concentrated core of a therapeutic agent that is surrounded by a polymeric shell or may have a solid or a liquid dispersed throughout a polymer matrix.

Lipids described herein can also be incorporated into emulsions, which are drug delivery vehicles that contain oil droplets or an oil core. An emulsion can be lipid-stabilized. For example, an emulsion may comprise an oil filled core stabilized by an emulsifying component such as a monolayer or bilayer of lipids.

Lipids described herein may be incorporated into a micelle. Micelles are self-assembling particles composed of amphipathic lipids or polymeric components that are utilized for the delivery of agents present in the hydrophobic core.

### Delivery of Nucleic Acid, Genetic Material, Proteins, Peptides or Other Charged Agents

Lipids disclosed herein may facilitate the incorporation of a compound or molecule (referred to herein also as “cargo” or “cargo molecule”) bearing a net negative or positive charge into the delivery vehicle and subsequent delivery to a target cell in vitro or in vivo. The cargo may include a complex, such as a gene editing complex.

In one embodiment, the cargo molecule is genetic material, such as a nucleic acid. The nucleic acid includes, without limitation, RNA, including small interfering RNA (siRNA), small nuclear RNA (snRNA), micro RNA (miRNA), messenger RNA (mRNA) or DNA such as vector DNA or linear DNA. The nucleic acid length can vary and can include nucleic acid of 5-50,000 nucleotides in length. The nucleic acid can be in any form, including single stranded DNA or RNA, double stranded DNA or RNA, or hybrids thereof. Single stranded nucleic acid includes antisense oligonucleotides.

In one embodiment, the cargo is an mRNA, which includes a polynucleotide that encodes at least one peptide, polypeptide or protein. The mRNA includes, but is not limited to, small activating RNA (saRNA) and trans-amplifying RNA (taRNA), as described in co-pending U.S. provisional Application No. 63/195,269, titled “mRNA Delivery Using Lipid Nanoparticles”, which is incorporated herein by reference.

The mRNA as used herein encompasses both modified and unmodified mRNA. In one embodiment, the mRNA comprises one or more coding and non-coding regions. The mRNA can be purified from natural sources, produced using recombinant expression systems and optionally purified, or may be chemically synthesized.

In those embodiments in which an mRNA is a chemically synthesized molecule, the mRNA can comprise nucleoside analogs such as analogs having chemically modified bases or sugars, and/or backbone modifications. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The mRNAs of the disclosure may be synthesized according to any of a variety of known methods. For example, mRNAs in certain embodiments may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor.

In some embodiments, in vitro synthesized mRNA may be purified before encapsulation to remove undesirable impurities including various enzymes and other reagents used during mRNA synthesis.

The present disclosure may be used to encapsulate mRNAs of a variety of lengths. In some embodiments, the present disclosure may be used to encapsulate in vitro synthesized mRNA ranging from about 1-20 kb, about 1-15 kb, about 1-10 kb, about 5-20 kb, about 5-15 kb, about 5-12 kb, about 5-10 kb, about 8-20 kb, or about 8-15 kb in length.

Typically, mRNA synthesis includes the addition of a “cap” on the 5′ end, and a “tail” on the 3′ end. The presence of the cap is important in providing resistance to nucleases found in most eukaryotic cells. The presence of a “tail” serves to protect the mRNA from exonuclease degradation.

In some embodiments, mRNAs include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

In a further embodiment, the mRNA is circular. Advantageously, such mRNA lacks 5′ and 3′ ends and thus may be more stable in vivo due to its resistance to degradation by exonucleases. The circular mRNA may be prepared by any known method, including any one of the methods described in Deviatkin et al., 2023, Vaccines, 11(2), 238, which is incorporated herein by reference. Translation of the circular mRNA is carried out by a cap-independent initiation mechanism.

While mRNA provided from in vitro transcription reactions may be desirable in certain embodiments, other sources of mRNA are contemplated, such as mRNA produced from bacteria, fungi, plants, and/or animals.

The mRNA sequence may comprise a reporter gene sequence, although the inclusion of a reporter gene sequence in pharmaceutical formulations for administration is optional. Such sequences may be incorporated into mRNA for in vitro studies or for in vivo studies in animal models to assess biodistribution.

In another embodiment, the cargo is an siRNA. An siRNA becomes incorporated into endogenous cellular machineries to result in mRNA breakdown, thereby preventing transcription. Since RNA is easily degraded, its incorporation into a delivery vehicle can reduce or prevent such degradation, thereby facilitating delivery to a target site.

The siRNA encompassed by embodiments of the disclosure may be used to specifically inhibit expression of a wide variety of target polynucleotides. The siRNA molecules targeting specific polynucleotides may be readily prepared according to procedures known in the art. An siRNA target site may be selected and corresponding siRNAs may be chemically synthesized, created by in vitro transcription, or expressed from a vector or PCR product. A wide variety of different siRNA molecules may be used to target a specific gene or transcript. The siRNA may be double-stranded RNA, or a hybrid molecule comprising both RNA and DNA, e.g., one RNA strand and one DNA strand. The siRNA may be of a variety of lengths, such as 15 to 30 nucleotides in length or 20 to 25 nucleotides in length. In certain embodiments, the siRNA is double-stranded and has 3′ overhangs or 5′ overhangs. In certain embodiments, the overhangs are UU or dTdT 3′. In particular embodiments, the siRNA comprises a stem loop structure.

In a further embodiment, the cargo molecule is a microRNA or small nuclear RNA. Micro RNAs (miRNAs) are short, noncoding RNA molecules that are transcribed from genomic DNA, but are not translated into protein. These RNA molecules are believed to play a role in regulation of gene expression by binding to regions of target mRNA. Binding of miRNA to target mRNA may downregulate gene expression, such as by inducing translational repression, deadenylation or degradation of target mRNA. Small nuclear RNA (snRNA) are typically longer noncoding RNA molecules that are involved in gene splicing. The snRNA molecules may have therapeutic importance in diseases that are an outcome of splicing defects.

In another embodiment, the cargo is a DNA vector as described in co-owned and co-pending U.S. Serial No. U.S. Application No. 63/202,210 titled “DNA Vector Delivery Using Lipid Nanoparticles”, which is incorporated herein by reference. The DNA vectors may be administered to a subject for the purpose of repairing, enhancing or blocking or reducing the expression of a cellular protein or peptide. Accordingly, the nucleotide polymers can be nucleotide sequences including genomic DNA, cDNA, or RNA.

As will be appreciated by those of skill in the art, the vectors may encode promoter regions, operator regions or structural regions. The DNA vectors may contain double-stranded DNA or may be composed of a DNA-RNA hybrid. Non-limiting examples of double-stranded DNA include structural genes, genes including operator control and termination regions, and self-replicating systems such as vector DNA.

Single-stranded nucleic acids include antisense oligonucleotides (complementary to DNA and RNA), ribozymes and triplex-forming oligonucleotides. In order to have prolonged activity, the single-stranded nucleic acids will preferably have some or all of the nucleotide linkages substituted with stable, non-phosphodiester linkages, including, for example, phosphorothioate, phosphorodithioate, phophoroselenate, or O-alkyl phosphotriester linkages.

The DNA vectors may include nucleic acids in which modifications have been made in one or more sugar moieties and/or in one or more of the pyrimidine or purine bases. Such sugar modifications may include replacement of one or more hydroxyl groups with halogens, alkyl groups, amines, azido groups or functionalized as ethers or esters. In another embodiment, the entire sugar may be replaced with sterically and electronically similar structures, including aza-sugars and carbocyclic sugar analogs. Modifications in the purine or pyrimidine base moiety include, for example, alkylated purines and pyrimidines, acylated purines or pyrimidines, or other heterocyclic substitutes known to those of skill in the art.

The DNA vector may be modified in certain embodiments with a modifier molecule such as a peptide, protein, steroid or sugar moiety. Modification of a DNA vector with such molecule may facilitate delivery to a target site of interest. In some embodiments, such modification translocates the DNA vector across a nucleus of a target cell. By way of example, a modifier may be able to bind to a specific part of the DNA vector (typically not encoding of the gene-of-interest), but also has a peptide or other modifier that has nucleus-homing effects, such as a nuclear localization signal. A non-limiting example of a modifier is a steroid-peptide nucleic acid conjugate as described by Rebuffat et al., 2002, Faseb J. 16(11):1426-8, which is incorporated herein by reference. The DNA vector may contain sequences encoding different proteins or peptides. Promoter, enhancer, stress or chemically-regulated promoters, antibiotic-sensitive or nutrient-sensitive regions, as well as therapeutic protein encoding sequences, may be included as required. Non-encoding sequences may be present as well in the DNA vector.

The nucleic acids used in the present method can be isolated from natural sources, obtained from such sources as ATCC or GenBank libraries or prepared by synthetic methods. Synthetic nucleic acids can be prepared by a variety of solution or solid phase methods. Generally, solid phase synthesis is preferred. Detailed descriptions of the procedures for solid phase synthesis of nucleic acids by phosphite-triester, phosphotriester, and H-phosphonate chemistries are widely available.

In one embodiment, the DNA vector is double stranded DNA and comprises more than 700 base pairs, more than 800 base pairs or more than 900 base pairs or more than 1000 base pairs.

In another embodiment, the DNA vector is a nanoplasmid or a minicircle.

Gene editing systems can also be incorporated into delivery vehicles comprising the charged lipid. This includes a Cas9-CRISPR, TALEN and zinc finger nuclease gene editing system. In the case of Cas9-CRISPR, a guide RNA (gRNA), together with a plasmid or mRNA encoding the Cas9 protein may be incorporated into a delivery vehicle comprising the lipids described herein. Optionally, a ribonucleoprotein complex may be incorporated into a delivery vehicle comprising the lipid described herein. Likewise, the disclosure includes embodiments in which genetic material encoding DNA binding and cleavage domains of a zinc finger nuclease or TALEN system are incorporated into a delivery vehicle together with the lipids of the disclosure.

While a variety of nucleic acid cargo molecules are described above, it will be understood that the above examples are non-limiting and the disclosure is not to be considered limiting with respect to the particular cargo molecule encapsulated in the delivery vehicle.

For example, the lipids described herein may also facilitate the incorporation of proteins and peptides into a delivery vehicle, which includes ribonucleoproteins. This includes both linear and non-linear peptides, proteins or ribonucleoproteins.

While pharmaceutical compositions are described above, the lipids described herein can be a component of any nutritional, cosmetic, cleaning or foodstuff product.

### Pharmaceutical Formulations

In some embodiments, the delivery vehicle comprising the cargo molecule is part of a pharmaceutical composition and is administered to treat and/or prevent a disease condition. The treatment may provide a prophylactic (preventive), ameliorative or a therapeutic benefit. The pharmaceutical composition will be administered at any suitable dosage.

In one embodiment, the pharmaceutical compositions is administered parentally, i.e., intra-arterially, intravenously, subcutaneously or intramuscularly. In yet a further embodiment, the pharmaceutical compositions are for intra-tumoral or in-utero administration. In another embodiment, the pharmaceutical compositions are administered intranasally, intravitreally, subretinally, intrathecally or via other local routes.

The pharmaceutical composition comprises pharmaceutically acceptable salts and/or excipients.

The compositions described herein may be administered to a patient. The term patient as used herein includes a human or a non-human subject.

The following examples are given for the purpose of illustration only and not by way of limitation on the scope of the invention.

### EXAMPLES

### Materials

The lipid 1,2-distearoyl-sn-glycero-3-phosphorylcholine (DSPC) and 1,2-dimyristoyl-rac-glycero-3-methoxypolyethylene glycol-2000 (PEG-DMG) were purchased from Avanti Polar Lipids (Alabaster, AL). Cholesterol and 10× Phosphate Buffered Saline (pH 7.4) were purchased from Sigma Aldrich (St Louis, MO). The ionizable amino-lipid was synthesized as previously described in U.S. Provisional Application No. 63/194,471 titled “Method for Producing an Ionizable Lipid”, which is incorporated herein by reference.

An mRNA encoding firefly luciferase purchased from RNA Technologies and Therapeutics (Montreal, QC) was used to analyse luciferase activity.

### Methods

Preparation of Lipid Nanoparticles (LNP) Containing mRNA

Lipids 1, 3-7, 16-19, 21-27 and 31-36 described herein, DSPC, cholesterol, and PEG-DMG, were dissolved in ethanol at the appropriate ratios to a final concentration of 10 mM total lipid. Nucleic acid (mRNA) was dissolved in an appropriate buffer such as 25 mM sodium acetate pH 4 or sodium citrate pH 4 to a concentration necessary to achieve the appropriate amine-to-phosphate ratios. The aqueous and organic solutions were mixed using a rapid-mixing device as described in Kulkarni et al., 2018, ACS Nano, 12:4787 and Kulkarni et al., 2017, Nanoscale, 36:133347 (each incorporated herein by reference) at a flow rate ratio of 3:1 (v/v; respectively) and a total flow rate of 20 mL/min. The resultant mixture was dialyzed directly against 1000-fold volume of PBS pH 7.4. All formulations were concentrated using an Amicon™ centrifugal filter unit and analysed using the methods described below.

### Analysis of LNP

Particle size analysis of LNPs in PBS was carried out using backscatter measurements of dynamic light scattering with a Malvern Zetasizer™ (Worcestershire, UK). The reported particle sizes correspond to the number-weighted average diameters (nm). Total lipid concentrations were determined by extrapolation from the cholesterol content, which was measured using the Cholesterol E-Total Cholesterol Assay (Wako Diagnostics, Richmond, VA) as per the manufacturer's recommendations. Encapsulation efficiency of the formulations was determined using the Quant-iT RiboGreen™ Assay kit (Invitrogen, Waltham, MA). Briefly, the total mRNA content in solution was measured by lysing lipid nanoparticles in a solution of TE containing 2% Triton Tx-100, and free DNA vector in solution (external to LNP) was measured based on the RiboGreen™ fluorescence in a TE solution without Triton. Total mRNA content in the formulation was determined using a modified Bligh-Dyer extraction procedure. Briefly, LNP formulations containing mRNA were dissolved in a mixture of chloroform, methanol, and PBS that results in a single phase and the absorbance at 260 nm measured using a spectrophotometer.

### In Vivo Analysis in CD-1 Mice

LNP-mRNA encoding firefly luciferase were injected intravenously (tail-vein) into 6-8 week old CD-1 mice. Four hours following injection, the animals were euthanized and the liver and spleen were isolated. Tissue was homogenized in Glo Lysis buffer and a luciferase assay performed using the Steady Glo Luciferase assay kit (as per manufacturers recommendations). The in vivo analysis of ionizable lipids 1, 5-7, 16-19, 21-27 and 31-36 in the liver and spleen was conducted in a set of experiments separate from ionizable lipids 3 and 4.

Organic Synthesis of the Lipids in this Disclosure.

Provided below are representative experimental procedures for the synthesis of certain lipids in Table 1. Those skilled in the art will appreciate that any other lipid in Table 1 and any congener of such lipids can be prepared by obvious modifications of the experimental procedures set forth herein.

Unless otherwise specified, all reagents and solvents were commercial products and were used without further purification, except THF (freshly distilled from Na/benzophenone under Ar), CH₂Cl₂ (freshly distilled from CaH₂ under Ar). “Dry methanol” was freshly distilled from magnesium turnings. All reactions were performed under an argon atmosphere. Reaction mixture from aqueous workups were dried by passing over a plug of anhydrous Na₂SO₄ held in a filter tube and concentrated under reduced pressure on a rotary evaporator. Thin-layer chromatography was performed on silica gel plates coated with silica gel (Merck 60 F254 plates) and column chromatography was performed on 230-400 mesh silica gel. Visualization of the developed chromatogram was performed by staining with 12 or potassium permanganate solution. ¹H and ¹³C nuclear magnetic resonance (NMR) spectra were recorded at room temperature in CDCl₃ solutions. ¹H NMR spectra were referenced to residual CHCl₃ (7.26 ppm) and ¹³C NMR spectra were referenced to the central line of the CDCl₃ triplet (77.00 ppm). Chemical shifts are reported in parts per million (ppm) on the 6 scale. Multiplicities are reported as “s” (singlet), “d” (doublet), “t” (triplet), “q” (quartet), “m” (multiplet), and further qualified as “app” (apparent) and “br” (broad). Low- and high-resolution mass spectra (m/z) were obtained in the electrospray (ESI) and field desorption/field ionisation (FD/FI) mode.

The synthesis of lipids 5-38 from caprolactone was carried out as set forth below. As discussed, the synthesis of said lipids involves subjecting certain esters or lactones to Claisen condensation under Mukaiyama conditions. This technology is as set forth in co-owned and co-pending WO 2023/147657 (incorporated herein by reference). The products of such Claisen reactions are subsequently converted into the final products as outlined in the Schemes above and as described below.

### Example 1: Methods for Chemically Synthesizing Ionizable Lipids

### A. Preparation of Building Blocks

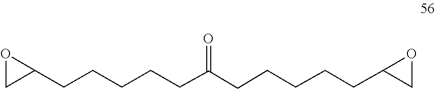 [SMILES: O=C(CCCCCC1CO1)CCCCCC1CO1]

(a) Pentadeca-1,14-dien-8-one (56). To a solution of methyl oct-7-enoate (6.55 g, 41.9 mmol) and NBu₃ (18.0 mL, 75.4 mmol) in toluene (80.0 mL) was added dropwise a solution of TiCl₄ (6.89 mL, 62.9 mmol) in toluene (40.0 mL) at 0° C. under an atmosphere of nitrogen. The reaction was warmed to room temperature and stirred for 2 hours. Water (40.0 mL) was added at 0° C. The biphasic mixture was extracted with toluene (2×40.0 mL). The combined organics were concentrated, the residue was dissolved in a EtOH (70.0 mL) and 25% NaOH (25.0 mL) was added. The mixture was stirred for 2 hours, concentrated to 25% volume, acidified to pH 2 with conc. HCl, and extracted with 50:50 Hexanes/Et₂O (3×40.0 mL). The combined organics were washed with brine, dried (Na₂SO₄) and concentrated. The residue was purified by silica chromatography (0-8% EtOAc in Hexanes) to yield 56 as an oil (4.24 g, 91%). ¹H NMR (400 MHz, CDCl₃) δ 5.79 (ddt, J=16.9, 10.2, 6.6 Hz, 2H), 5.07-4.84 (m, 4H), 2.38 (t, J=7.4 Hz, 4H), 2.10-1.99 (m, 4H), 1.62-1.52 (m, 4H), 1.45-1.34 (m, 4H), 1.34-1.23 (m, 4H).

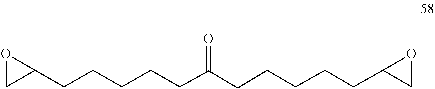 [SMILES: O=C(CCCCCC1CO1)CCCCCC1CO1]

(b). 1,11-di(Oxiran-2-yl)undecan-6-one (58). Solid mCPBA (13.9 g, ˜50% pure) was added to a solution of ketone 37 (4.20 g, 19.9 mmol) in DCM (70.0 mL) at 0° C. The mixture was warmed to room temperature and stirred for 2 hours. The mixture was cooled to 0° C. and quenched with sat. aq. sodium sulfite and diluted with water (20.0 mL). The layers were separated, and the organics were washed with 1 N NaOH (3×30.0 mL), dried (Na2SO4) and concentrated to yield the bis-epoxide 43 as a waxy white solid (4.32 g, 90%). ¹H NMR (400 MHz, CDCl₃) δ 2.94-2.83 (m, 2H), 2.76-2.69 (m, 2H), 2.44 (dd, J=5.0, 2.7 Hz, 2H), 2.38 (td, J=7.5, 1.6 Hz, 4H), 1.65-1.20 (m, 16H).

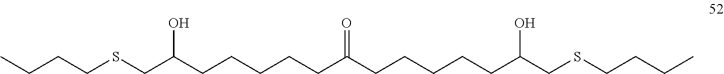 [SMILES: CCCCSCC(O)CCCCCC(=O)CCCCCC(O)CSCCCC]

(c) 1,15-bis(Hexylthio)-2,14-dihydroxypentadecan-8-one (52). To a well-stirred solution of bis-epoxide 58 (2.5 g, 10 mmol) and 1-hexanethiol (2.6 g, 22 mmol, 2.2 equiv) in EtOH (50 mL) maintained under inert atmosphere was added solid NaOH (1.6 g, 40 mmol, 4 equiv). The mixture was heated at reflux for 2 hours, cooled, diluted with water (20 mL) and extracted with DCM (3×15.0 mL). The combined organics were dried (NaSO₄) and concentrated. The residue was purified by silica chromatography (0-50% EtOAc in hexanes) to yield 52 (3.9 g, 80%). ¹H NMR (400 MHz, CDCl₃) δ 3.69-3.55 (m, 2H), 2.73 (dd, J=13.6, 3.3 Hz, 2H), 2.51 (t, J=7.4 Hz, 4H), 2.46-2.34 (m, 6H), 1.67-1.23 (m, 32H), 0.90 (t, J=7.0 Hz, 6H).

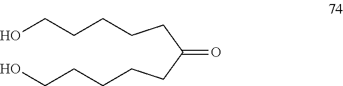 [SMILES: O=C(CCCCCO)CCCCCO]

(d) 1,11-Dihydroxyundecan-6-one (74). A solution of TiCl₄ (74.8 g, 44 mL, 0.39 mol) in CH₂Cl₂ (200 mL) was added dropwise to a cold (0° C., ice bath), stirred solution of caprolactone (30 g, 0.26 mol) and triethylamine (Et₃N) (47.2 g, 67 mL, 0.46 mol) in CH₂Cl₂ (450 mL). After completion of the reaction, water (40 mL) was added and all volatiles were removed on rotatory evaporator (bath temperature 60° C.). The residue was extracted with several portions of 10% MeOH in CH₂Cl₂, and the combined extracts were dried over anhydrous Na₂SO₄. Evaporation of the solvent resulted in a yellowish oily residue, which was purified by column chromatography on silica gel (230-400 mesh) by eluting with a gradient of 5→7% MeOH in DCM. This provided 17.1 g of product (65% yield) as an a off white solid. ¹H NMR (400 MHz, CDCl₃) δ 3.61-3.64 (t, 3H, J=13.2 Hz), 2.40-2.44 (t, 3H, J=14.8 Hz), 2.04 (br s, 2H), δ 1.53-1.63 (m, 8H), 1.31-1.39 (m, 6=4H). ¹³C NMR (100 MHz, CDCl₃) δ 211.7, 62.5, 42.6, 32.3, 25.3, 23.4.

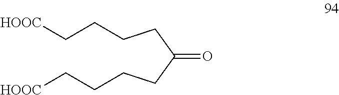 [SMILES: O=C(O)CCCCC(=O)CCCCC(=O)O]

(e) 6-Oxoundecanedioic acid (94). A solution of commercial monoethyl adipate, 90 (5.20 g, 29.9 mmol), in SOCl₂ (5.50 mL) was heated to reflux for 2 minutes then cooled to room temperature. Excess SOCl₂ was removed under vacuum. The residue was dissolved in toluene (5.00 mL) and concentrated to remove any remaining SOCl₂, yielding the crude acid chloride 91 (5.73 g, quantitative), which was used in the next step without purification. ¹H NMR (400 MHz, CDCl₃) δ 4.14 (q, J=7.2 Hz, 2H), 2.92 (t, J=7.0 Hz, 2H), 2.33 (t, J=7.1 Hz, 2H), 1.87-1.60 (m, 4H), 1.26 (t, J=7.2 Hz, 3H). Neat Et₃N (4.15 mL, 29.7 mmol) was added dropwise over the course of 3 minutes to a stirring solution of the above acid chloride (5.73 g, 29.7 mmol) in toluene (50.0 mL) at 0° C. under an atmosphere of nitrogen. The reaction was warmed to 35° C. and stirred for 15 minutes, then cooled to room temperature and stirred for an additional 30 minutes, at which point a thick white precipitate had formed. The mixture was filtered through a pad of Celite,® and the solid precipitate was washed with more toluene (15.0 mL). The combined filtrates were concentrated to yield 5-(3-(4-ethoxy-4-oxobutyl)-4-oxooxetan-2-ylidene)pentanoate, 92, which was used directly in the next step without further purification. ¹H NMR (400 MHz, CDCl₃) δ 4.75 (dt, 1H, J₁=7.7, J₂=1.3 Hz), 4.15 (AA′BB′, 4H, app J₁=7.1 Hz), 3.99 (br t, 1H, J=6.9 Hz), 2.40-2.29 (m, 4H), 2.19 (br q, 2H, J=7.5 Hz), 1.89-1.64 (m, 6H), 1.27 (t, 6H, J=7.1 Hz). This compound was suspended in 2 N aq. KOH (25.0 mL) and heated at reflux for 6 hours, whereupon the solution became homogenous. The cooled solution was washed with Et₂O (2×15.0 mL), and the ether extracts were discarded. The solution was then acidified with conc. HCl to pH 2. The aqueous layer was then kept at 0′C for 1 hour, during which time a precipitate formed. The solid was collected by suction filtration to yield 6-oxo-undecanedioic acid, 94, as an off white solid (2.8 g, 62% over two steps). ¹H NMR (400 MHz, CDCl₃) δ 2.44-2.38 (m, 4H), 2.37-2.30 (m, 4H), 1.69-1.55 (m, 8H). MS (negative ion ESI): m/z 229 [M−1]⁻.

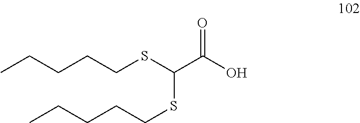 [SMILES: CCCCCSC(SCCCCC)C(=O)O]

(f) 2,2-bis(Pentylthio)acetic acid (102). A solution of glyoxylic acid monohydrate (1.00 g, 10.9 mmol), 1-pentanethiol (2.97 mL, 23.9 mmol) and TsOH (188 mg, 1.09 mmol) in toluene (40.0 mL) was refluxed under inert atmosphere for 5 hours. The mixture was then cooled to room temperature and washed with water (30.0 mL) and brine (30.0 mL). The organic layer was dried (Na₂SO₄) and concentrated to yield 102 (2.80 g, quantitative) which was used in the next step without further purification. ¹H NMR (400 MHz, CDCl3) δ 4.37 (s, 1H), 2.82-2.61 (m, 4H), 1.62 (p, J=7.4 Hz, 4H), 1.47-1.27 (m, 8H), 0.90 (t, J=7.0 Hz, 6H).

### (g) 9-(((Pentylthio)methyl)thio)nonanoic Acid (103)

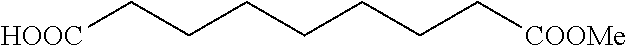 [SMILES: COC(=O)CCCCCCCC(=O)O]

(i) Monomethyl azelate. A solution of dimethyl azelate (50.0 g, 231.5 mmol, 1 equiv) and Ba(OH)₂ (43.8 g, 138.6 mmol, 1.2 equiv OH) in methanol (300 mL) was stirred at room temperature overnight. A heavy white precipitate formed. The solid was collected by vacuum filtration and washed with fresh MeOH (3×30 mL). The solid was suspended in 4N aq. HCl solution (200 mL) and the resulting mixture was extracted with CH₂Cl₂ (4×60 mL). The combined extracts were washed with brine (2×50 mL), dried (Na₂SO₄) and evaporated to give 43.0 g of crude product (92%), which was used directly in the next step. ¹H NMR (300 MHz, CDCl₃): δ 11.60 (s, 1H) 3.55 (s, 3H), 2.21 (m, 4H), 1.52 (m, 4H), 1.22 (m, 6H). ¹³C NMR (75 MHz, CDCl₃): δ 179.6, 174.1, 51.2, 33.7 (2 peaks), 28.6 (3 peaks) 24.6, 24.3.

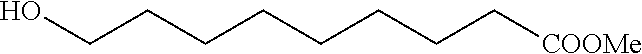 [SMILES: COC(=O)CCCCCCCCO]

(ii) Methyl 9-hydroxynonanoate. To a cold (0° C.) THF (100 mL) solution of monomethyl azelate (12.5 g, 61.8 mmol), maintained under N₂, in was added BH₃-DMS (7.32 mL, 80.4 mmol). The mixture was warmed to room temperature and stirred for 90 minutes, at which point ¹H NMR confirmed consumption of starting material. The mixture was cooled to 0° C. and quenched carefully with H₂O (20.0 mL). The mixture was then extracted with DCM (3×40.0 mL). The combined organics were washed (brine), dried (Na₂SO₄), and concentrated to yield methyl 9-hydroxynonanoate (10.9 g, 94%). ¹H NMR: δ 3.64 (s, 3H), 3.60 (t, J=6.8 Hz, 2H), 2.27 (t, J=1.2 Hz, 2H), 1.62-1.47 (m, 4H), 1.28 (s, 8H). ¹³C NMR: 6 174.2, 62.9, 51.4, 34.0, 32.7, 29.1 (2 peaks), 29.0, 25.6, 24.8. LRMS (ESI) 201 [M+Na]⁺.

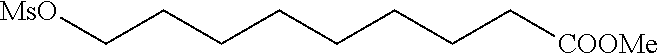 [SMILES: COC(=O)CCCCCCCCOS(C)(=O)=O]

(iii) Methyl 9-((methylsulfonyl)oxy)nonanoate. To a solution of methyl 9-hydroxynonanoate (10.2 g, 54.1 mmol) and TEA (10.0 mL, 71.7 mmol) in DCM (70.0 mL) was added MsCl (5.03 mL, 65.0 mmol) at 0° C. under an atmosphere of nitrogen. The mixture was warmed to rt and stirred for 18 hours, at which point it was diluted with water (40.0 mL) and extracted with DCM (3×50.0 mL). The combined organics were washed (brine), dried (Na₂SO₄), and concentrated to yield the crude mesylate (14.2 g, quant.), which was advanced to the next step without purification. ¹H NMR. δ 4.21 (t, J=6.5 Hz, 2H), 3.66 (s, 3H), 3.00 (s, 3H), 2.30 (t, J=7.5 Hz, 2H), 1.73 (dt, J=8.3, 6.5 Hz, 2H), 1.65-1.56 (m, 2H), 1.45-1.24 (m, 8H).

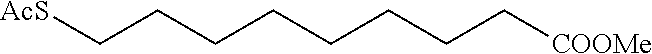

(iv) Methyl 9-(acetylthio)nonanoate. To a slurry of K₂CO₃ (13.3 g, 96.3 mmol) and crude mesylate above (14.2 g) in THF (100 mL) maintained under N₂ atmosphere, was added thioacetic acid (6.11 mL, 86.8 mmol) at rt. The mixture was refluxed for 48 hours, cooled to room temperature and diluted with water (100 mL). The mixture was extracted with DCM (3×100 mL). The combined organics were washed (brine), dried (Na₂SO₄), and concentrated to yield the crude thiacetate (13 g), which was advanced to the next step without purification. ¹H NMR (400 MHz, CDCl₃) δ 3.66 (s, 3H), 2.85 (t, J=7.4 Hz, 2H), 2.32 (s, 3H), 2.28 (t, J=7.7 Hz, 2H), 1.68-1.47 (m, 4H), 1.38-1.23 (m, 8H).

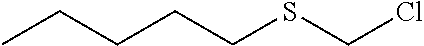

(v) (Chloromethyl)(pentyl)sulfane. Gaseous HCl was bubbled through a cold (−15° C., ice/salt bath) solution of 1-pentanethiol (0.23 g, 2.2 mmol) in dry DCM (2 mL) containing suspended paraformaldehyde (0.10 g, 3.3 mmol) and maintained under argon (balloon, needle vent). The mixture was stirred for 2 hours at −15° C. The solvent was removed under reduced pressure and DI H₂O was added to the residue until all the solid dissolved (ca. 5 mL). The mixture was extracted with Et₂O (3×5 mL) and the combined extracts were sequentially washed with saturated aqueous sodium bicarbonate solution (3×5 mL), water (3×5 mL), saturated aqueous sodium chloride solution (2×5 mL), dried over Na₂SO₄ and concentrated on a rotary evaporator to afford 0.34 g (quantitative) of chloromethyl sulfane as a colorless oil. This compound is a sensitive material that is best used without purification. ¹H NMR (300 MHz, CDCl₃) □ 4.77 (s, 2H), 2.76 (t, 2H), 1.68 (m, 2H), 1.39 (m, 4H), 0.93 (t, 3H) ppm. ¹³C NMR (75 MHz, CDCl₃) □ 49.9, 31.6, 30.9, 28.3, 22.2, 13.9.

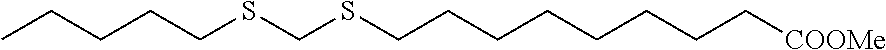 [SMILES: CCCCCSCSCCCCCCCCC(=O)OC]

(vi) Methyl 9-(((pentylthio)methyl)thio)nonanoate. The thioacetate of part (iv) above (25.0 g, 101 mmol) was added via syringe to a degassed (N₂ bubbling for 20 min), well-stirred solution of NaOMe (5.5 g, 101 mmol) in DMF (500 mL) at room temperature. After stirring for 20 minutes, no more thioacetate was apparent by TLC. The chloromethyl sulfane of part (v) (15.5 g, 101 mmol) was cautiously added via syringe to the well-stirred solution. The reaction was complete after 45 min, whereupon it was quenched with water and extracted with hexanes. The layers were separated, and the organic phase was collected. The aqueous phase was extracted with hexanes (2×50 mL). The combined extracts were dried (Na₂SO₄), filtered and evaporated to yield the crude product (29.4 g, 90%) as a yellow oil. This material can be advanced to the following step, but if desired, it can be further purified by silica chromatography (1.5% EtOAc in hexanes). ¹H NMR: δ 3.66 (s, 3H), 3.65 (s, 2H), 2.62 (td, J=7.4, 1.4 Hz, 4H), 2.25 (t, J=7.5 Hz, 2H), 1.65-1.52 (m, 8H), 1.48-1.26 (m, 10H), 0.90 (t, J=7.1 Hz, 3H).

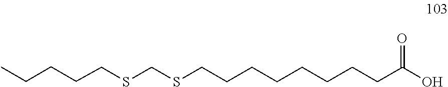 [SMILES: CCCCCSCSCCCCCCCCC(=O)O]

(vii) 9-(((Pentylthio)methyl)thio)nonanoic acid (103). To a solution of methyl 9-(pentylsulfanylmethylsulfanyl)nonanoate (2.7 g, 8.42 mmol) in ethanol (20 mL) was added NaOH (1.12 g, 28.1 mmol) dissolved in 10 mL water. The reaction mixture was stirred at reflux under inert atmosphere. The reaction was completed in 3 hours. The reaction was quenched with 1 M HCl and extracted with ethyl acetate. The combined organic layer was dried over Na₂SO₄ and concentrated under reduced pressure to afford the product, which was used without purification. ¹H NMR (400 MHz, CDCl₃): δ 3.65 (s, 2H), 2.62 (tr, J=7.4 Hz, 4H), 2.35 (t, J=7.5 Hz, 2H), 1.67-1.54 (m, 6H), 1.43-1.28 (m, 12H), 0.90 (t, J=7.1 Hz, 3H).

(h) Preparation of various fragments: compounds 104-109. A solution of a carboxylic acid (5 mmol, 1.0 equiv) in CH₂Cl₂ (1.5 mL) was added to a solution of an aminoacid methyl ester hydrochloride (5.5 mmol, 1.1 equiv), DMAP (2.5 mmol, 0.5 equiv), N,N-diisopropylethylamine (7.5 mmol, 1.5 equiv), and EDCI-HCl (7.5 mmol, 1.5 equiv) in CH₂Cl₂ (8 mL), under Ar. The resulting mixture was stirred overnight at room temperature, then it was diluted with CH₂Cl₂ (12 mL), sequentially washed with sat. aq. NaHCO₃ solution (2×5 mL), H₂O (2×5 mL), sat. aq. NaCl solution (2×5 mL), dried (Na₂SO₄) and concentrated under reduced pressure. This residue was purified by silica gel column chromatography with 10%-15% EtOAc/hexanes to provide the desired N-acyl derivative (65-75% yield) as a colorless oil. This material (3 mmol) was dissolved in ethanol (6 mL) and treated with 20% aq. NaOH solution (1 mL) at rt and under argon. Upon complete saponification of the ester (TLC, NMR), the solution was acidified to pH 1 with 4N aq. HCl and concentrated. The aqueous residue was extracted with DCM (3×5 mL). The combined extracts were dried (Na₂SO₄) and concentrated to afford the crude acid (92-98%), which was used without purification. The following compound were thus prepared:

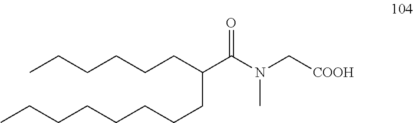 [SMILES: CCCCCCCCC(CCCCCC)C(=O)N(C)CC(=O)O]

(i) N-(2-Hexyldecanoyl)-N-methylglycine (104). ¹H NMR (400 MHz, CDCl₃; 1:2 mixture of rotamers) δ 4.16 & 4.10 (s, 2H, rotamers, 1:2), 3.12 & 3.00 (s, 3H, rotamer, 1:2), 2.40-2.45 (m, 1H), 1.90-1.28 (br m, 24H), 0.87-0.91 (m, 6H). LRMS: 328 [M+H]⁺.

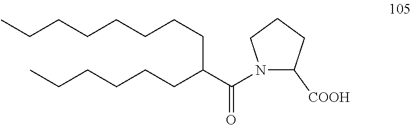 [SMILES: CCCCCCCCC(CCCCCC)C(=O)N1CCCC1C(=O)O]

(ii) (2-Hexyldecanoyl)proline (105). ¹H NMR (400 MHz, CDCl₃; 1:2 mixture of rotamers) δ4.51-4.45 (m, 1H), 3.71-3.63 (m, 1H), 3.61-3.51 (m, 1H), 2.49-2.43 (m, 1H), 2.23-1.75 (m, 4H), 1.68-1.59 (m, 4H), 1.49-1.24 (m, 20H), 0.89-0.85 (m, 6H). LRMS: 354 [M+H]⁺

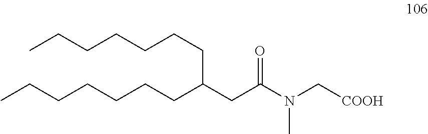 [SMILES: CCCCCCCC(CCCCCCC)CC(=O)N(C)CC(=O)O]

(iii) N-(3-heptyldecanoyl)-N-methylglycine (106). ¹H NMR (400 MHz, CDCl₃; 1:3 mixture of rotamers) δ 4.16, 4.10 (s, 2H, rotamers, 1:3), 3.12, 3.00 (s, 3H, rotamer, 1:3), 2.31-2.33 (m, 2H), 1.91-1.93 (br m, 1H), 1.28 (br s, 24H), 0.87-0.91 (m, 6H). LRMS: 342 [M+H]⁺.

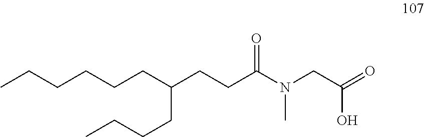 [SMILES: CCCCCCC(CCCC)CCC(=O)N(C)CC(=O)O]

(iv) N-(4-Butyldecanoyl)-N-methylglycine (107). ¹H NMR (400 MHz, CDCl₃, rotamers): δ 9.72 (br, 1H), 4.15-4.08 (s, 2H), 3.10 and 2.98 (s, 3H), 2.41-2.18 (m, 2H), 1.79-1.48 (m, 3H), 1.35-1.19 (m, 16H), 0.92-0.83 (m, 6H).

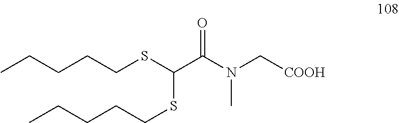 [SMILES: CCCCCSC(SCCCCC)C(=O)N(C)CC(=O)O]

(v) N-(2,2-bis(Pentylthio)acetyl)-N-methylglycine (108). ¹H NMR (400 MHz, CDCl₃, rotamers) δ 4.71 & 4.58 (s, 1H, rotamers, 1:3), 4.35 & 4.17 (s, 2H, rotamers, 1:3), 3.13 (d, J=78.5 Hz, 3H), 2.83-2.57 (m, 4H), 1.59 (m, 4H), 1.42-1.21 (m, 8H), 0.90 (td, J=7.1, 3.2 Hz, 6H).

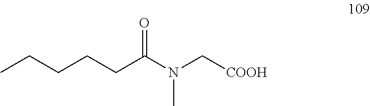

(vi) N-hexanoyl-N-methylglycine (109). ¹H NMR (400 MHz, CDCl₃, rotamers): δ 4.13 (m, 2H), 3.12 & 2.89 (s, 3H, rotamer), 2.41 & 2.33 (s, 2H, rotamer), 1.70-1.54 (m, 2H), 1.37-1.21 (m, 4H), 0.87 (m, 3H).

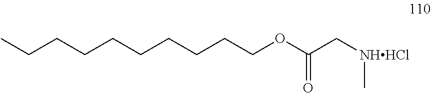

(vii) n-Decyl N-methylglycinate hydrochloride (110). To a solution of N-BOC sarcosine (1.9 g, 10 mmol, 1 equiv), decanol (2.0 g, 13 mmol, 1.3 equiv), and 4-dimethylaminopyridine (860 mg, 7 mmol, 0.7 equiv), in CH₂Cl₂ (25 mL) was added 1-ethyl-3-(3-dimethylaminopropyl) carbodiimide (1.5 equiv). The mixture was stirred for 16 h at rt under nitrogen, whereupon TLC indicated completion. The solution was diluted with ice water (100 mL) and acidified to pH 4 with 1N aq. HCl. The CH₂Cl₂ layers were separated and the aqueous layer was extracted with more CH₂Cl₂ (3×20 mL). The combined organic phases were dried (Na₂SO₄), filtered, and concentrated under reduced pressure. The residue was purified by chromatography on silica gel (20-30% EtOAc in hexane) to give pure N-BOC ester product (2.4 g, 73%). ¹H NMR (400 MHz, CDCl₃) δ 4.16-4.06 (m, 2H), 3.96 & 3.87 (s, 2H, rotamers), 2.92 & 2.90 (s, 3H, rotamers), 1.68-1.55 (m, 2H), 1.41 & 1.59 (s, 9H, rotamers), 1.34-1.17 (m, 14H), 0.86 (t, J=6.84 Hz, 3H). LRMS: 352 [M+Na]⁺. A solution of this material (1 g, 3.0 mmol) in CH₂Cl₂ (2 mL) was treated with 4M HCl in dioxane (1 mL). After 20 min, the volatiles were removed under vacuum and the residue of 110 was thoroughly dried under high vacuum and used as such without purification. ¹H NMR (400 MHz, CDCl₃) δ 4.16 (t, J=6.79 Hz, 2H), 3.83 (s, 2H), 2.80 (s, 3H), 1.62 (p, J=6.77 Hz, 2H), 1.42-1.07 (m, 14H), 0.85 (t, J=6.75 Hz, 3H). LRMS: 230 [M+H]⁺.

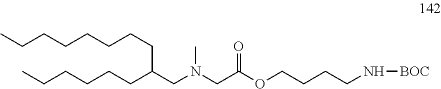 [SMILES: CCCCCCCCC(CCCCCC)CN(C)CC(=O)OCCCCNC(=O)OC(C)(C)C]

(viii) 4-((tert-Butoxycarbonyl)amino)butyl N-(2-hexyldecanoyl)-N-methylglycinate (142). A solution of N-(2-hexyldecanoyl)-N-methylglycine acid (1.2 g, 3.7 mmol), tert-butyl (4-hydroxybutyl)carbamate (693 mg, 3.7 mmol), EDCI-HCl (1.1 g, 5.55 mmol) and DMAP (227 mg, 1.85 mmol) in DCM (15.0 mL) was stirred under inert atmosphere for 18 hours then concentrated. The residue was purified by silica chromatography (0-20% EtOAc in Hexanes) to yield 142 (1.3 g, 72%) as an oil. ¹H NMR (400 MHz, CDCl₃) δ 4.11-3.90 (m, 4H), 3.29-2.94 (m, 5H, rotamers), 2.66-2.61 (m, 1H), 1.74-1.48 (m, 10H), 1.38 (s, 9H), 1.20-1.16 (m, 20H), 0.83-0.85 (m, 6H).

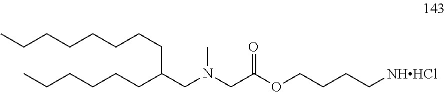

(ix) 4-Aminobutyl N-(2-hexyldecanoyl)-N-methylglycinate hydrochloride (143). To a cold (0° C.), well-stirred solution of 142 (1.0 g, 2.0 mmol) in CH₂Cl₂ (5 mL) maintained under nitrogen was added 4N HCl in dioxane until TLC showed complete disappearance of the starting material. The mixture was evaporated to dryness and the crude residue of 143 (870 mg, ˜quant) was used without purification. ¹H NMR (400 MHz, CDCl₃, rotamers) δ 8.75 (br s, 2H) 4.0-3.85 (m, 4H), 3.02 & 2.99 (m, 5H, rotamers, 1:3), 2.44-2.41 (m, 1H), 1.88-1.42 (m, 8H), 1.20-1.16 (m, 20H), 0.83-0.85 (m, 6H).

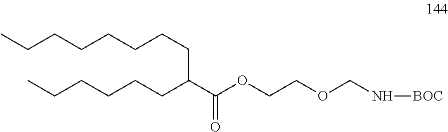 [SMILES: CCCCCCCCC(CCCCCC)C(=O)OCCOCNC(=O)OC(C)(C)C]

(x) 4-((tert-Butoxycarbonyl)amino)butyl 2-hexyldecanoate (144). A solution of 2-hexyldecanoic acid (1 g, 3.9 mmol), tert-butyl (4-hydroxybutyl)carbamate (738 mg, 3.9 mmol), EDCI-HCl (1.2 g, 5.85 mmol) and DMAP (240 mg, 1.95 mmol) in DCM (15.0 mL) was stirred under inert atmosphere for 18 hours then concentrated. The residue was purified by silica chromatography (0-20% EtOAc in Hexanes) to yield 144 (1.1 g, 84%) as an oil. ¹H NMR (400 MHz, CDCl₃) δ 4.56 (s, 1H), 4.10 (t, J=6.4 Hz, 2H), 3.17 (d, J=6.8 Hz, 2H), 2.33 (tt, J=9.1, 5.3 Hz, 1H), 1.75-1.51 (m, 7H), 1.50-1.39 (m, 9H), 1.27 (s, 21H), 0.92-0.85 (m, 6H).

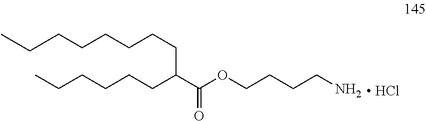 [SMILES: CCCCCCCCC(CCCCCC)C(=O)OCCCCN ; "· HCl"]

(xi) 4-Aminobutyl 2-hexyldecanoate hydrochloride (145). Prepared according to procedure ix above. ¹H NMR (400 MHz, CDCl₃) δ 8.35 (s br, 2H), 4.10 (t, J=6.4 Hz, 2H), 3.06-3.03 (m, 2H), 2.34-2.27 (m, 1H), 1.88-1.75 (m, 4H), 1.60-1.25 (m, 24H), 0.87 (t, J=6.7 Hz, 6H).

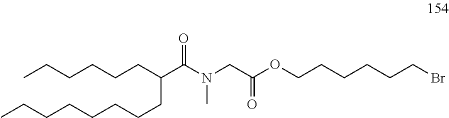 [SMILES: CCCCCCCCC(CCCCCC)C(=O)N(C)CC(=O)OCCCCCCBr]

(xii) 6-Bromohexyl N-(2-hexyldecanoyl)-N-methylglycinate (155). A solution of acid 104 (811 mg, 2.48 mmol), 6-bromo-1-hexanol (582 mg, 3.22 mmol), EDCI-HCl (665 mg, 3.47 mmol) and DMAP (424 mg, 3.47 mmol) in DCM (10.0 mL) was stirred under inert atmosphere for 18 hours then concentrated. The residue was purified by silica chromatography (0-20% EtOAc in Hexanes) to yield 103 (900 mg, 74%) as an oil. ¹H NMR (400 MHz, CDCl₃, rotamers) δ4.19-4.12 (m, 4H), 3.44-3.41 (m, 2H), 3.13 & 3.02 (s, 3H, rotamers, 3:1), 2.72-2.67 (m, 1H), 1.91-1.84 (m, 2H), 1.74-1.62 (m, 4H), 1.53-1.34 (m, 6H), 1.27 (br s, 20H), 0.98-0.84 (m, 6H). LRMS: 490 & 492 [M+H]+

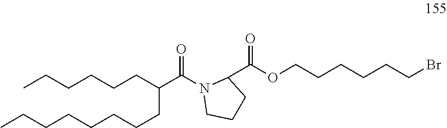 [SMILES: CCCCCCCCC(CCCCCC)C(=O)N1CCCC1C(=O)OCCCCCCBr]

(xiii) 6-Bromohexyl (2-hexyldecanoyl)prolinate (156). Prepared from acid 105 and 6-bromo-1-hexanol according to procedure xii above. ¹H NMR (400 MHz, CDCl₃, rotamers) δ4.51-4.45 (m, 1H), 4.21-4.03 (m, 2H), 3.71-3.63 (m, 1H), 3.61-3.51 (m, 1H), 3.43-3.39 (m, 2H), 2.49-2.43 (m, 1H), 2.23-1.75 (m, 6H), 1.68-1.59 (m, 4H), 1.49-1.34 (m, 6H), 1.26-1.24 (br m, 20H), 0.89-0.85 (m, 6H). LRMS: 516 & 518 [M+H]⁺.

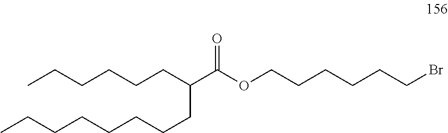 [SMILES: CCCCCCCCC(CCCCCC)C(=O)OCCCCCCBr]

(xiv) 6-Bromohexyl 2-hexyldecanoate (157). Prepared from 2-hexyldecanoic acid and 6-bromo-1-hexanol according to procedure xii above. ¹H NMR δ 4.09 (t, 2H, J=6.5), 3.43 (t, 2H, J=6.8), 2.36-2.29 (m, 1H), 1.92-1.85 (m, 2H), 1.69-1.36 (m, 9H), 1.27 (br s, 21H) 0.88 (t, 6H, J=6.2 Hx). LRMS: m/z 419 [M+H]⁺.

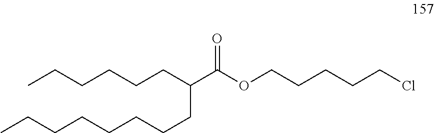 [SMILES: CCCCCCCCC(CCCCCC)C(=O)OCCCCCCl]

(xv) 5-Chloropentyl 2-hexyldecanoate (158). Prepared from 2-hexyldecanoic acid and 5-chloro-1-pentanol according to procedure xii above. ¹NMR (400 MHz, CDCl₃) δ 4.12-4.08 (t, 2H), 5.67-3.543 (t, 2H), 2.36-2.29 (m, 1H), 1.79-1.86 (m, 2H), 1.71-1.42 (m, 8H), 1.27 (br s, 22H), 0.91-0.87 (m, 6H).

### C. Preparation of Ketone Precursors to Lipids 5-38

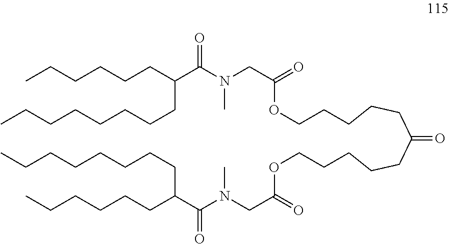 [SMILES: CCCCCCCCC(CCCCCC)C(=O)N(C)CC(=O)OCCCCCC(=O)CCCCCOC(=O)CN(C)C(=O)C(CCCCCC)CCCCCCCC]

(a) Procedure for the deesterification of a dihydroxyketone: 6-oxoundecane-1,11-diyl bis(2-(2-hexyl-N-methyldecanamido)acetate) (115). To a solution of 1,11-dihydroxyundecan-6-one, 74 (202 mg, 1 mmol, 1 equiv), and N-(2-hexyldecanoyl)-N-methylglycine, 104 (655 mg, 2 mmol, 2 equiv), in CH₂Cl₂ (2.5 mL) was added a solution of T3P (50% in EtOAc, 1.5 mL, 800 mg of T3P, 2.5 equiv) and pyridine (316 mg, 322 μL, 4 mmol, 4 equiv). The mixture was stirred for 16 h at rt under nitrogen, whereupon TLC indicated completion. The solution was diluted with ice water (10 mL) and extracted with CH₂Cl₂ (3×15 mL). The combined extracts were dried (Na₂SO₄), filtered, and concentrated under reduced pressure. The residue was purified by chromatography on silica gel (20-30% EtOAc in hexane) to give pure product (63%) as a colorless oil. ¹H NMR (400 MHz, CDCl₃, rotamers) δ4.16-4.03 (m, 8H), 3.12 & 3.01 (s, 6H, rotamer, 1:3), 2.72-2.65 (m, 2H), 2.42-2.38 (m, 4H), 1.66-1.56 (m, 12H), 1.46-1.26 (m, 48H), 0.90-0.84 (m, 12H). LRMS: 821 [M+H]⁺.

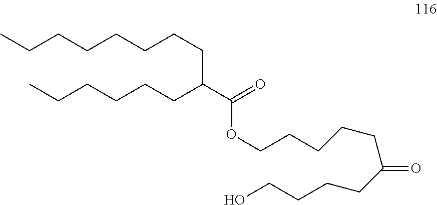 [SMILES: CCCCCCCCC(CCCCCC)C(=O)OCCCCCC(=O)CCCCO]

(b) Procedure for the monoesterification of a dihydroxyketone: 11-hydroxy-6-oxoundecyl 2-hexyldecanoate (116). To a solution of 1,11-dihydroxyundecan-6-one, 74 (6.40 g, 31.6 mmol, 1.0 equiv), in DCM (50 mL) was added a solution of 2-hexyldecanoic acid (8.11 g, 31.6 mmol, 1.0 equiv) in DCM (20 mL), 1-(3-dimethylaminopropyl)-3-ethylcarbodiimide hydrochloride (9.06 g, 47.3 mmol, 1.5 equiv), and 4-dimethylaminopyridine (2.7 g, 22.1 mmol, 0.7 equiv). After stirring the reaction mixture at room temperature and under inert atmosphere overnight, the reaction was diluted with DCM (30 mL) and washed with H₂O (3×30 mL). The combined organic layers were dried over Na₂SO₄ and concentrated under reduced pressure to afford a yellow oil as the crude product. The crude mixture was purified by silica gel chromatography with 0-65% EtOAc/hexanes to provide the desired product (55-60% yield). 1HNMR (400 MHz, CDCl₃) δ: 4.05 (tr, 2H), 3.64 (tr, 2H), 2.43-2.36 (m, 4H), 2.34-2.24 (m, 1H), 1.67-1.45. 2.40 (m, 10H), 1.45-1.19 (m, 26H), 0.86 (tr, 6H).

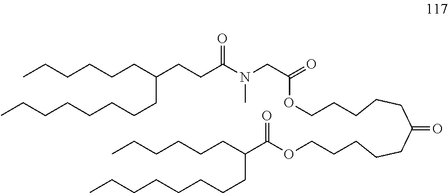 [SMILES: CCCCCCCCC(CCCCCC)CCC(=O)N(C)CC(=O)OCCCCCC(=O)CCCCCOC(=O)C(CCCCCC)CCCCCCCC]

(c) Preparation of an unsymmetrical diester derivative of a dihydroxy-ketone: 11-((N-(2-hexyldecanoyl)-N-methylglycyl)oxy)-6-oxoundecyl 2-hexyldecanoate (117). To a solution of 11-hydroxy-6-oxoundecyl 2-hexyldecanoate, 116, (220 mg, 0.5 mmol), N-(2-hexyldecanoyl)-N-methylglycine, 104 (182 mg, 0.55 mmol, 1.1 equiv), and 4-dimethylaminopyridine (82 mg, 0.7 mmol, 0.7 equiv), in CH₂Cl₂ (3 mL) was added 1-ethyl-3-(3-dimethylaminopropyl)carbodiimide (EDCI, 1.5 equiv) and stirred for 16 h at rt under nitrogen, whereupon the reaction was complete (TLC). The volatiles were removed under vacuum and the residue was purified by chromatography on silica gel (10-20% EtOAc in hexane) to give diester 117 (307 mg, 82%) as a colorless gummy oil. ¹H NMR (400 MHz, CDCl₃, rotamers) δ4.15-4.05 (m, 6H), 3.12 & 3.01 (s, 3H, rotamers, 3:1), 2.72-2.65 (m, 1H), 2.44-2.39 (m, 4H), 2.36-2.28 (m, 1H), 1.74-1.54 (m, 12H), 1.46-1.26 (m, 48H), 0.90-0.84 (m, 12H). LRMS: 750 [M+H]⁺.

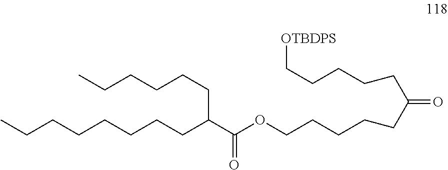

(d) 11-((tert-Butyldiphenylsilyl)oxy)-6-oxoundecyl 2-hexyldecanoate (118). A solution of tert-butyl(chloro)-diphenylsilane (TBDPSCl; 1.1 g, 3.75 mmol, 1.1 equiv) in CH₂Cl₂ (4 mL) was added dropwise during 15 min to a well-stirred solution of 11-hydroxy-6-oxoundecyl 2-hexyldecanoate, 116 (1.5 g, 3.4 mmol, 1.0 equiv), and imidazole (508 mg, 7.48 mmol, 2.2 equiv) in DCM (15 mL). The mixture was stirred overnight at room temperature. The reaction mixture was sequentially washed with sat. aq. NaHCO₃ solution (2×10 mL), water (2×10 mL), and sat. aq. NaCl chloride solution (2×10 mL), then dried over anhydrous Na₂SO₄, filtered, and concentrated under reduced pressure to furnish 118 (2.1 g, 91%) as a colorless oil. ¹H NMR (400 MHz, CDCl₃) δ 7.78-7.63 (m, 4H), 7.48-7.33 (m, 6H), 4.08 (t, J=6.6 Hz, 2H), 3.67 (t, J=6.4 Hz, 2H), 2.46-2.26 (m, 5H), 1.70-1.51 (m, 10H), 1.51-1.17 (m, 26H), 1.06 (s, 9H), 0.94-0.85 (m, 6H).

(e) The following unsymmetrical diesters were prepared from appropriate acids and alcohols according to procedures (b) and (c) above:

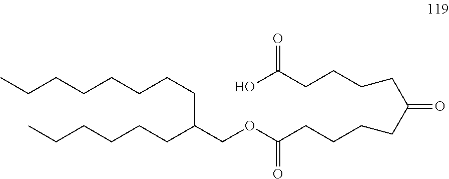 [SMILES: CCCCCCCCC(CCCCCC)COC(=O)CCCCC(=O)CCCCC(=O)O]

(i) 11-((2-Hexyldecyl)oxy)-6,11-dioxoundecanoic acid (119). Obtained from 6-oxoundecanedioic acid, 94, and 2-hexyldecan-1-ol according to procedure (b) above. ¹H NMR (400 MHz, CDCl₃) δ 3.95 (d, J=5.80 Hz, 2H), 2.46-2.25 (m, 8H), 1.66-1.51 (m, 9H), 1.34-1.19 (m, 24H), 0.86 (t, J=6.70 Hz, 6H). LRMS: 455 [M+H]⁺.

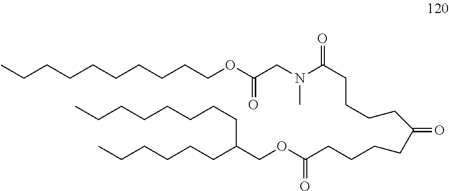 [SMILES: CCCCCCCCCCOC(=O)CN(C)C(=O)CCCCC(=O)CCCCC(=O)OCC(CCCCCC)CCCCCCCC]

(ii) 11-(Methyl(2-oxo-2-(tetradecyloxy)ethyl)amino)-6,11-dioxoundecanoate (120). Prepared from 119 and 110 according to procedure (c) above. ¹H NMR (400 MHz, CDCl₃) δ 4.22-3.99 (m, 4H), 3.95 (d, J=5.79 Hz, 2H), 3.06 & 2.96 (s, 3H, rotamers), 2.49-2.15 (m, 8H), 1.68-1.54 (m, 11H), 1.37-1.18 (m, 38H), 0.87 (t, J=6.7 Hz, 9H). LRMS: 666 [M+H]⁺.

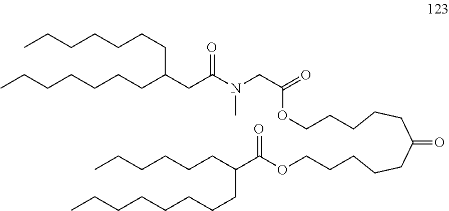 [SMILES: CCCCCCCCC(CCCCCC)C(=O)OCCCCCC(=O)CCCCCOC(=O)CN(C)C(=O)CC(CCCCCCC)CCCCCCC]

(iii) 11-((N-(3-Heptyldecanoyl)-N-methylglycyl)oxy)-6-oxoundecyl 2-hexyldecanoate (123). ¹H NMR (400 MHz, CDCl₃, rotamers) δ 4.19-4.01 (m, 6H), 3.07 & 2.97 (s, 3H, rotamers, 1:4), 2.40 (t, 4H), 2.28 and 2.13 (m, 3H), 1.92 (m, 1H), 1.69-1.51 (m, 12H), 1.35-1.16 (m, 48H), 0.87 (br t, 12H)

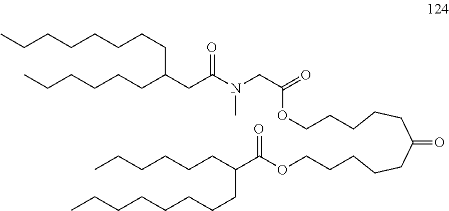 [SMILES: CCCCCCCCC(CCCCCC)CC(=O)N(C)CC(=O)OCCCCCC(=O)CCCCCOC(=O)C(CCCCCC)CCCCCCCC]

(iv) 11-((N-(3-Hexylundecanoyl)-N-methylglycyl)oxy)-6-oxoundecyl 2-hexyldecanoate (124). ¹H NMR (400 MHz, CDCl₃, rotamers) δ4.19-4.01 (m, 6H), 3.07 and 2.97 (s, 3H), 2.40 (t, J=7.4, 4H), 2.34-2.11 (m, 3H), 1.92 (m, 1H), 1.69-1.51 (m, 11H), 1.48-1.18 (m, 49H), 0.90-0.81 (tr, 12H).

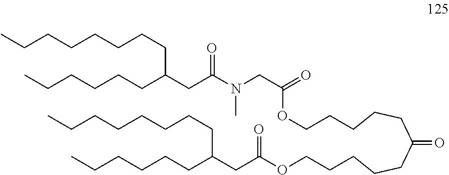 [SMILES: CCCCCCCCC(CCCCCC)CC(=O)OCCCCCC(=O)CCCCCOC(=O)CN(C)C(=O)CC(CCCCCC)CCCCCCCC]

(v) 11-((N-(3-Hexylundecanoyl)-N-methylglycyl)oxy)-6-oxoundecyl 3-hexylundecanoate (125). ¹H NMR (400 MHz, CDCl₃, rotamers) δ4.20-4.00 (m, 6H), 3.07 and 2.97 (s, 3H), 2.40 (t, 4H), 2.32-2.09 (m, 4H), 1.93 (m, 1H), 1.83 (m, 1H), 1.68-1.52 (m, 10H), 1.38-1.18 (m, 50H), 0.91-0.83 (m, 12H).

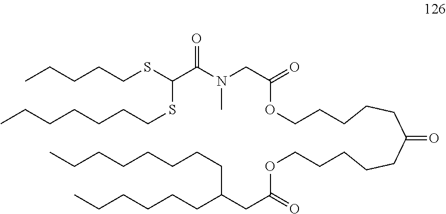 [SMILES: CCCCCCCCC(CCCCCC)CC(=O)OCCCCCC(=O)CCCCCOC(=O)CN(C)C(=O)C(SCCCCC)SCCCCCCC]

(vi) 11-((N-(2,2-bis(Pentylthio)acetyl)-N-methylglycyl)oxy)-6-oxoundecyl 3-hexylundecanoate (126). ¹H NMR (400 MHz, CDCl₃, rotamers) δ 4.71 & 4.56 (s, 1H, rotamers, 1:3), 4.30-3.99 (m, 6H), 3.22 & 3.03 (s, 3H, rotamers, 1:4), 2.81-2.56 (m, 4H), 2.42 (t, J=7.4 Hz, 4H), 2.23 (d, J=6.9 Hz, 2H), 1.87 (d, J=6.3 Hz, 1H), 1.73-1.51 (m, 12H), 1.42-1.19 (m, 36H), 0.90 (td, J=7.0, 4.7 Hz, 12H).

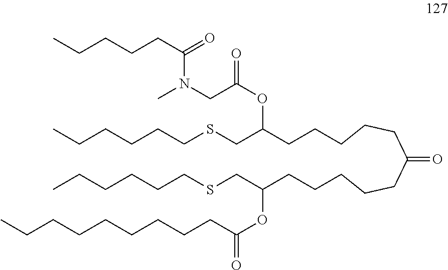 [SMILES: CCCCCCCCCC(=O)OC(CCCCCC(=O)CCCCCC(CSCCCCCC)OC(=O)CN(C)C(=O)CCCCC)CSCCCCCC]

(vii) 14-((N-Hexanoyl-N-methylglycyl)oxy)-1,15-bis(hexylthio)-8-oxopentadecan-2-yl decan-oate (127). ¹H NMR (400 MHz, CDCl₃, rotamers) δ5.10-4.87 (m, 2H), 4.23-4.00 (m, 2H), 3.07 and 2.98 (s, 3H), 2.67-2.59 (m, 4H), 2.56-2.48 (m, 4H), 2.41-2.21 (m, 8H), 1.76-1.49 (m, 16H), 11.40-1.19 (m, 36H), 0.93-0.82 (t, 12H)

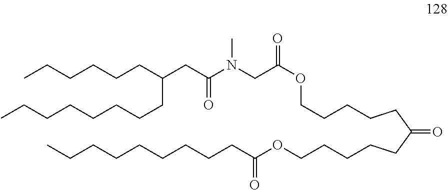 [SMILES: CCCCCCCCCC(=O)OCCCCCC(=O)CCCCCOC(=O)CN(C)C(=O)CC(CCCCCC)CCCCCCCC]

(viii) 11-((N-(3-Hexylundecanoyl)-N-methylglycyl)oxy)-6-oxoundecyl decanoate (128). ¹H NMR (400 MHz, CDCl₃, rotamers) δ 4.19-4.01 (m, 6H), 3.07 and 2.96 (s, 3H), 2.40 (t, 4H), 2.28 (m, 4H), 1.91 (m, 1H), 1.69-1.53 (m, 10H), 1.39-1.18 (m, 40H), 0.87 (t, 9H).

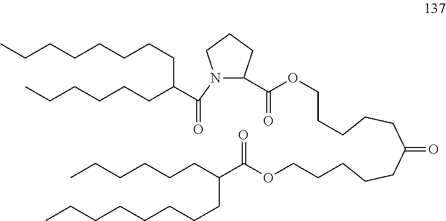 [SMILES: CCCCCCCCC(CCCCCC)C(=O)OCCCCCC(=O)CCCCCOC(=O)C1CCCN1C(=O)C(CCCCCC)CCCCCCCC]

(viii) 11-((2-Hexyldecanoyl)oxy)-6-oxoundecyl (2-hexyldecanoyl)prolinate (137). ¹H NMR (400 MHz, CDCl₃, rotamers) δ4.51-4.46 (m, 1H), 4.18-4.05 (m, 4H), 3.71-3.54 (m, 2H), 2.49-2.39 (m, 5H), 2.35-2.28 (m, 1H), 2.23-1.85 (m, 4H), 1.74-1.50 (m, 13H), 1.39-1.27 (m, 47H), 0.90-0.84 (m, 12H). LRMS: 776 [M+H]+

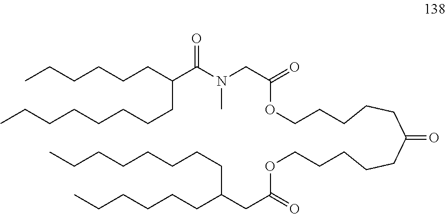 [SMILES: CCCCCCCCC(CCCCCC)CC(=O)OCCCCCC(=O)CCCCCOC(=O)CN(C)C(=O)C(CCCCCC)CCCCCCCC]

(ix) 11-((N-(2-Hexyldecanoyl)-N-methylglycyl)oxy)-6-oxoundecyl 3-hexylundecanoate (138). ¹H NMR (400 MHz, CDCl₃, rotamers) δ4.18-4.00 (m, 6H), 3.10 and 2.99 (s, 3H), 2.67 (m, 1H), 2.39 (t, J=7.4, 4H), 2.21 (d, J=6.9, 2H), 1.82 (m, 1H), 1.71-1.53 (m, 10H), 1.45-1.16 (m, 50H), 0.91-0.82 (m, 12H).

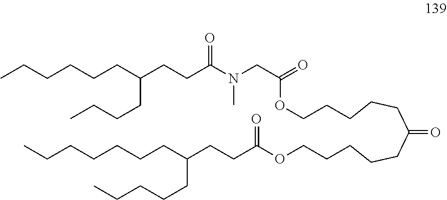 [SMILES: CCCCCCCC(CCCCC)CCC(=O)OCCCCCC(=O)CCCCCOC(=O)CN(C)C(=O)CCC(CCCC)CCCCCC]

(x) 11-((N-(4-Butyldecanoyl)-N-methylglycyl)oxy)-6-oxoundecyl 4-pentylundecanoate (139). ¹H NMR (400 MHz, CDCl₃, rotamers) δ4.18-4.00 (m, 6H), 3.08 and 2.97 (s, 3H), 2.45-2.15 (m, 8H), 1.71-1.53 (m, 12H), 1.38-1.16 (m, 42H), 0.91-0.82 (t, 12H).

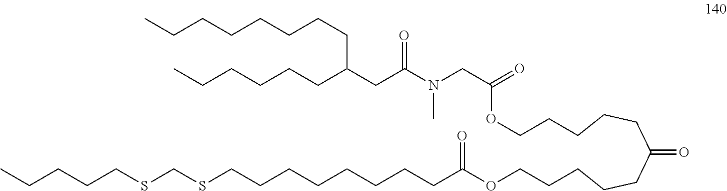 [SMILES: CCCCCCCCC(CCCCCC)CC(=O)N(C)CC(=O)OCCCCCC(=O)CCCCCOC(=O)CCCCCCCCSCSCCCCC]

(xi) 11-((N-(3-hexylundecanoyl)-N-methylglycyl)oxy)-6-oxoundecyl 9-(((pentylthio)methyl)-thio)nonanoate (140). ¹H NMR (400 MHz, CDCl₃, rotamers) δ4.18-4.02 (m, 6H), 3.65 (s, 2H), 3.07 and 2.97 (s, 3H), 2.61 (tr, 4H), 2.40 (tr, 4H), 2.31-2.24 (m, 4H), 1.93 (m, 1H), 1.70-1.52 (m, 10H), 1.43-1.19 (m, 38H), 0.92-0.82 (m, 9H).

### D. Synthesis of Lipids 5-38

(a) Procedure for the Conversion of a Ketone into a Lipid with a Type 1 Head Group.

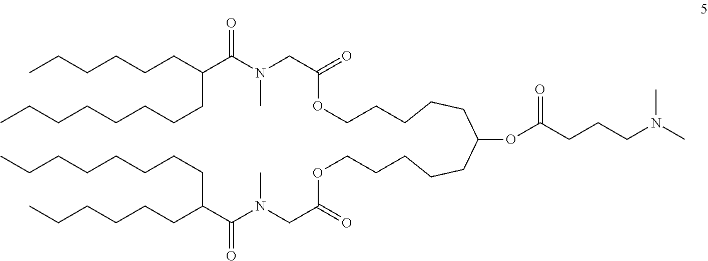 [SMILES: CCCCCCCCC(CCCCCC)C(=O)N(C)CC(=O)OCCCCCC(CCCCCOC(=O)CN(C)C(=O)C(CCCCCC)CCCCCCCC)OC(=O)CCCN(C)C]

(i) 6-hydroxyundecane-1,11-diyl bis(2-(2-hexyl-N-methyldecanamido)acetate) (121) and 6-((4-(dimethylamino)butanoyl)oxy)undecane-1,11-diyl bis(2-(2-hexyl-N-methyldecanamido)-acetate) (5). and Solid NaBH₄ (50 mg, 1.3 mmol) was added to a solution of ketone 115 (1.0 g, 1.2 mmol) in isopropanol (1 mL) and the solution was stirred at room temperature under a nitrogen atmosphere for 45 min, whereupon the reduction was complete (TLC, ¹H NMR). Aqueous saturated NH₄Cl solution (1 mL) was carefully added dropwise to the well-stirred solution (foaming, H₂ evolution). When gas evolution subsided, he mixture was diluted with water (1 mL) and extracted with CH₂Cl₂ (3×5 mL). The combined extracts were dried (Na₂SO₄) and concentrated to afford crude 121 (quant.), which was immediately used in the next step without purification. To a solution of alcohol 121 (assume 1.2 mmol, 1 equiv) and 4-(dimethylamino)butyric acid hydrochloride (240 mg, 1.44 mmol, 1.2 equiv) in CH₂Cl₂ (3 mL) were added N-(3-dimethylaminopropyl)-N′-ethylcarbodiimide hydrochloride (344 mg, 1.5 equiv) and DMAP (102 mg, 0.7 equiv). The reaction mixture was stirred at room temperature for 16 hours. The reaction mixture was diluted with CH₂Cl₂ (50 mL) and washed with saturated NaHCO₃ aq. (50 mL). The organic layer was dried (Na₂SO₄), filtered and concentrated. The residue was purified by silica gel column chromatography (1-2% MeOH in CH₂Cl₂) to give title compound (90%) as a colorless oil. ¹H NMR (400 MHz, CDCl₃, rotamers) δ4.88-4.85 (m, 1H), 4.18-4.09 (m, 8H), 3.12 & 3.01 (s, 6H, rotamers, 3:1), 2.79-2.61 (m, 9H), 2.44-2.40 (m, 2H), 2.03 (m, 2H), 1.74-1.49 (m, 13H), 1.45-1.26 (m, 52H), 0.90-0.86 (m, 12H).

The following lipids were prepared by the same method:

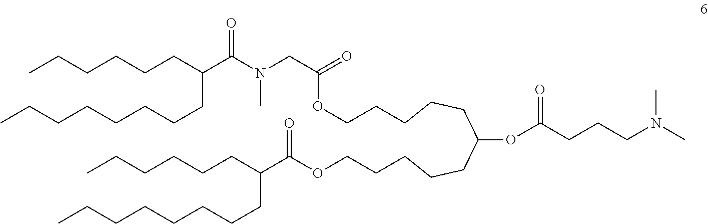 [SMILES: CCCCCCCCC(CCCCCC)C(=O)OCCCCCC(CCCCCOC(=O)CN(C)C(=O)C(CCCCCC)CCCCCCCC)OC(=O)CCCN(C)C]

(ii) 6-((4-(Dimethylamino)butanoyl)oxy)-11-((N-(2-hexyldecanoyl)-N-methylglycyl)oxy)-undecyl 2-hexyldecanoate (6). From ketone 117. ¹H NMR (400 MHz, CDCl₃, rotamers) δ4.95-4.81 (m, 1H), 4.16-4.04 (m, 6H), 3.13 & 3.02 (s, 3H, rotamers, 3:1), 2.72-2.66 (m, 1H), 2.49-2.26 (m, 11H), 1.96-1.84 (m, 2H), 1.64-1.26 (m, 64H), 0.90-0.84 (br t, 12H).

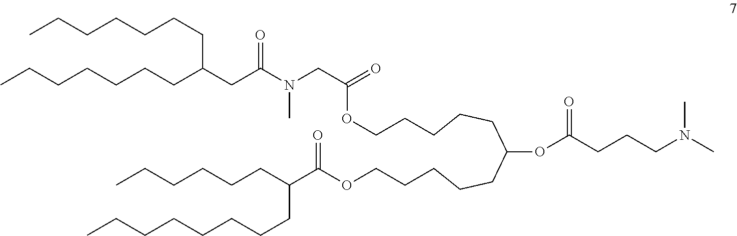 [SMILES: CCCCCCCCC(CCCCCC)C(=O)OCCCCCC(CCCCCOC(=O)CN(C)C(=O)CC(CCCCCCC)CCCCCCC)OC(=O)CCCN(C)C]

(iii) 6-((4-(Dimethylamino)butanoyl)oxy)-11-((N-(3-heptyldecanoyl)-N-methylglycyl)oxy)-undecyl 2-hexyldecanoate (7). From ketone 123. ¹H NMR (400 MHz, CDCl₃, rotamers) δ 4.86 (m, 1H), 4.17-0 4.00 (m, 6H), 3.07 and 2.97 (s, 3H), 2.38-2.09 (m, 12H), 1.92 (m, 1H), 1.80 (m, 2H), 1.67-1.16 (m, 65H), 0.91-0.82 (br t, 12H).

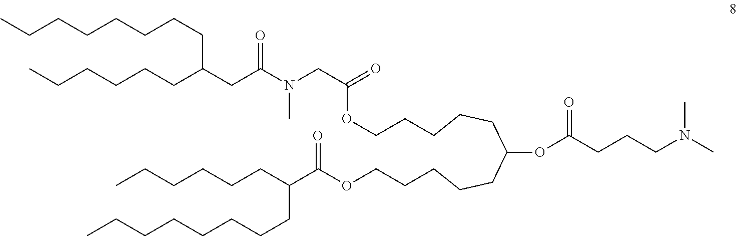 [SMILES: CCCCCCCCC(CCCCCC)CC(=O)N(C)CC(=O)OCCCCCC(CCCCCOC(=O)C(CCCCCC)CCCCCCCC)OC(=O)CCCN(C)C]

(iv) 6-((4-(Dimethylamino)butanoyl)oxy)-11-((N-(3-hexylundecanoyl)-N-methylglycyl)oxy)-undecyl 2-hexyldecanoate (8). From ketone 124. ¹H NMR (400 MHz, CDCl₃, rotamers) δ 4.86 (m, 1H), 4.15-4.00 (m, 6H), 3.07 and 2.96 (s, 3H), 2.37-2.24 (m, 6H), 2.22 (s, 6H), 1.92 (m, 1H), 1.78 (m, 2H), 1.65-1.47 (m, 10H), 1.45-1.14 (m, 55), 0.89-0.83 (br t, 12H)

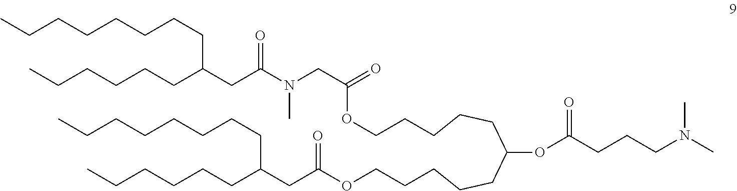 [SMILES: CCCCCCCCC(CCCCCC)CC(=O)OCCCCCC(CCCCCOC(=O)CN(C)C(=O)CC(CCCCCC)CCCCCCCC)OC(=O)CCCN(C)C]

(v) 6-((4-(dimethylamino)butanoyl)oxy)-11-((N-(3-hexylundecanoyl)-N-methylglycyl)oxy)-undecyl 3-hexylundecanoate (9). From ketone 125. ¹H NMR (400 MHz, CDCl₃, rotamers) δ 4.86 (m, 1H), 4.18-4.00 (m, 6H), 3.07 and 2.97 (s 3H), 2.39-2.10 (m, 12H), 1.97-1.7 4 (m, 4H), 1.65-1.47 (m, 10H), 1.40-1.14 (m, 56H), 0.91-0.81 (br t, 12H).

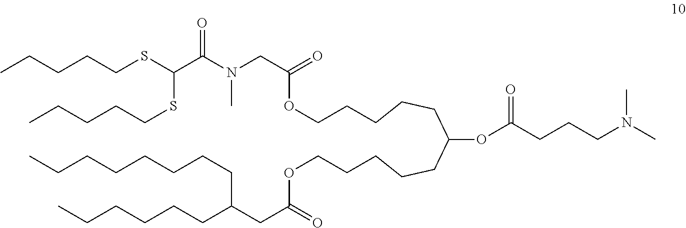 [SMILES: CCCCCCCCC(CCCCCC)CC(=O)OCCCCCC(CCCCCOC(=O)CN(C)C(=O)C(SCCCCC)SCCCCC)OC(=O)CCCN(C)C]

(vi) 11-((N-(2,2-bis(pentylthio)acetyl)-N-methylglycyl)oxy)-6-((4-(dimethylamino)butanoyl)-oxy)undecyl 3-hexylundecanoate (10). From ketone 126. ¹H NMR (400 MHz, CDCl₃, rotamers) δ 4.88 (m, 1H), 4.71 & 4.56 (s, 1H, rotamers, 3:1), 4.29-3.99 (m, 6H), 3.21 & 3.03 (s, 3H, rotamers, 3:1), 2.83-2.54 (m, 4H), 2.36 (td, J=7.4, 5.5 Hz, 4H), 2.28 (d, J=1.9 Hz, 6H), 2.23 (d, J=6.9 Hz, 2H), 1.83 (p, J=7.5 Hz, 3H), 1.69-1.45 (m, 12H), 1.45-1.12 (m, 40H), 0.95-0.84 (br t, 12H).

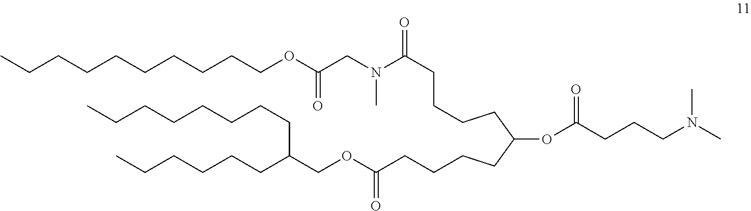 [SMILES: CCCCCCCCCCOC(=O)CN(C)C(=O)CCCCC(CCCCC(=O)OCC(CCCCCC)CCCCCCCC)OC(=O)CCCN(C)C]

(vii) 2-Hexyldecyl 6-((4-(dimethylamino)butanoyl)oxy)-11-(methyl(2-oxo-2-(tetradecyloxy)-ethyl)amino)-11-oxoundecanoate (11). From ketone 120. ¹H NMR (400 MHz, CDCl₃, rotamers) δ5.00-4.73 (m, 1H), 4.24-3.99 (m, 4H), 3.95 (d, J=5.82 Hz, 2H), 3.05 & 2.95 (s, 3H, rotamers), 2.40-2.17 (m, 14H), 1.86-1.74 (m, 2H), 1.70-1.47 (m, 11H), 1.36-1.18 (m, 42H), 0.87 (br t, J=6.71 Hz, 9H). LRMS: 781 [M+H]⁺.

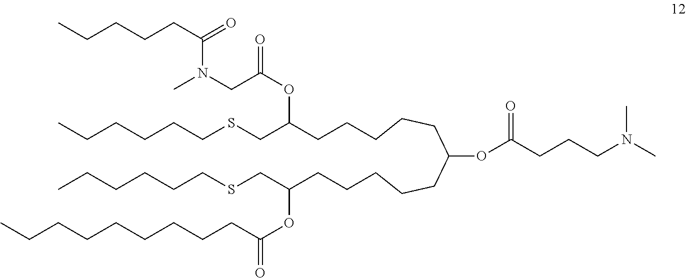 [SMILES: CCCCCCCCCC(=O)OC(CCCCCC(CCCCCC(CSCCCCCC)OC(=O)CN(C)C(=O)CCCCC)OC(=O)CCCN(C)C)CSCCCCCC]

(viii) 8-((4-(Dimethylamino)butanoyl)oxy)-14-((N-hexanoyl-N-methylglycyl)oxy)-1,15-bis-(hexylthio)pentadecan-2-yl decanoate (12). From ketone 127. ¹H NMR (400 MHz, CDCl₃, rotamers) δ5.09-4.89 (m, 2H), 4.84 (m, 1H), 4.23-4.02 (m, 2H), 3.07 and 2.98 (s, 3H), 2.68-2.60 (m, 4H), 2.56-2.46 (m, 4H), 2.40-2.21 (m, 12H), 1.81 (m, 2H), 1.76-1.42 (m, 18H), 1.42-1.17 (m, 40H), 0.93-0.83 (tr, J=7.1 Hz, 12H).

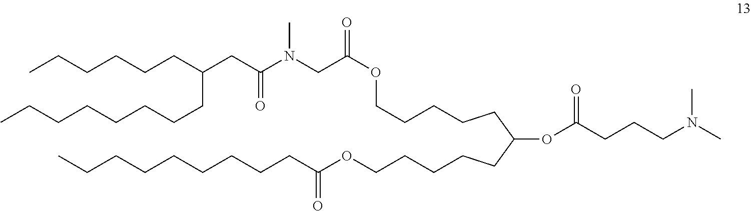 [SMILES: CCCCCCCCCC(=O)OCCCCCC(CCCCCOC(=O)CN(C)C(=O)CC(CCCCCC)CCCCCCCC)OC(=O)CCCN(C)C]

(ix) 6-((4-(Dimethylamino)butanoyl)oxy)-11-((N-(3-hexylundecanoyl)-N-methylglycyl)-oxy)-undecyl decanoate (13). From ketone 128. ¹H NMR (400 MHz, CDCl₃, rotamers) δ 4.86 (m, 1H), 4.18-4.00 (m, 6H), 3.07 and 2.97 (s, 3H), 2.45-2.08 (m, 14H), 1.96-1.78 (m, 3H), 1.67-1.46 (m, 10H), 1.37-1.20 (m, 44H), 0.92-0.81 (br t, 9H).

(b) Procedure for the Conversion of a Ketone into a Lipid with a Type 2 Head Group.

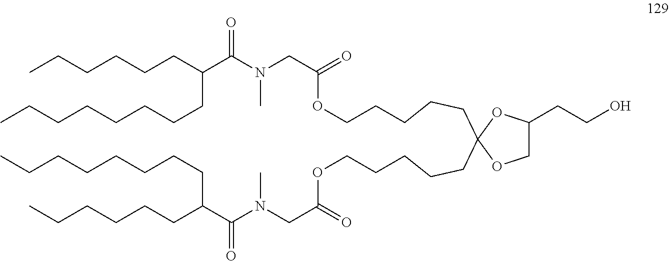 [SMILES: CCCCCCCCC(CCCCCC)C(=O)N(C)CC(=O)OCCCCCC1(CCCCCOC(=O)CN(C)C(=O)C(CCCCCC)CCCCCCCC)OCC(CCO)O1]

(i) (4-(2-hydroxyethyl)-1,3-dioxolane-2,2-diyl)bis(pentane-5,1-diyl) bis(2-(2-hexyl-N-methyldecan-amido)acetate) (129). A solution of ketone 115 (1.6 g, 2 mmol, 1 equiv), 1,2,4-butanetriol (320 mg, 3 mmol, 1.5 equiv) and pyridinium p-toluenesulfonate (150 mg, 0.6 mmol, 0.3 equiv) in toluene (35 mL) was refluxed under nitrogen overnight with continuous removal of water (Dean-Stark trap). The mixture was cooled to room temperature, washed with water (2×10 mL), brine (10 mL) then dried (Na₂SO₄) and concentrated. The residue was purified by silica gel column chromatography (20-30% EtOAc in hexane) to yield ketal 129 (52%) as an oil. ¹H NMR (400 MHz, CDCl₃, rotamers) δ4.24-4.09 (m, 10H), 3.82-3.79 (m, 2H), 3.55-3.13 (m, 1H), 3.13 & 3.01 (s, 6H, rotamers 1:3), 2.72-2.68 (m, 2H), 1.84-1.70 (m, 3H), 1.64-1.53 (m, 12H), 1.42-1.22 (m, 52H), 0.97-0.85 (m, 12H).

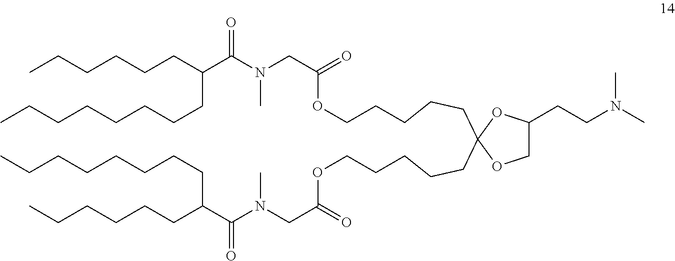 [SMILES: CCCCCCCCC(CCCCCC)C(=O)N(C)CC(=O)OCCCCCC1(CCCCCOC(=O)CN(C)C(=O)C(CCCCCC)CCCCCCCC)OCC(CCN(C)C)O1]

(ii) (4-(2-(dimethylamino)ethyl)-1,3-dioxolane-2,2-diyl)bis(pentane-5,1-diyl) bis(2-(2-hexyl-N-methyldecanamido)acetate) (14). A solution of ketal 129 (910 mg, 1 mmol, 1 eq.), TEA (151 mg, 210 μL, 1.5 mmol, 1.5 eq.), DMAP (25 mg, 0.2 mmol, 0.2 eq.) and TsCl (230 mg, 1.2 mmol, 1.2 eq.) in DCM (4 mL) was stirred at rt under nitrogen until completion (TLC). The reaction was quenched with water (5 mL) and extracted with DCM. The combined organics were dried (Na₂SO₄) and concentrated to yield the crude tosylate 130 (quantitative), which was used in the next step without purification. A solution of crude tosylate 130 and dimethylamine (2 M in THF, 2 mL) in MeOH (10 mL) was heated in a microwave reactor (110° C., normal absorbance) for 15 minutes. The mixture was then concentrated, and the residue purified by silica chromatography (0-5% MeOH in DCM) to yield lipid 14 (91% over 2 steps) as an oil. ¹H NMR (400 MHz, CDCl₃, rotamers) δ 4.19-4.05 (m, 10H), 3.50-3.46 (m, 1H), 3.12 & 3.02 (s, 3H, rotamers 3:1), 2.72-2.66 (m, 2H), 2.42-2.25 (m, 8H), 1.82-1.50 (m, 13H), 1.46-1.26 (m, 56H), 0.90-0.84 (m, 12H). LRMS: 936 [M+H]⁺.

The following compounds were prepared by the same method:

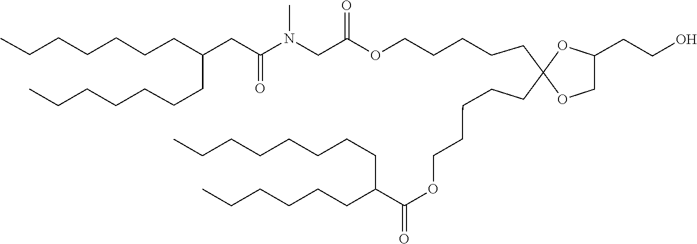 [SMILES: CCCCCCCCC(CCCCCC)C(=O)OCCCCCC1(CCCCCOC(=O)CN(C)C(=O)CC(CCCCCCC)CCCCCCC)OCC(CCO)O1]

(iii) 5-(2-(5-((N-(3-Heptyldecanoyl)-N-methylglycyl)oxy)pentyl)-4-(2-hydroxyethyl)-1,3-dioxolan-2-yl)pentyl 2-hexyldecanoate. From ketone 123. ¹H NMR (400 MHz, CDCl₃, rotamers) δ4.29-4.02 (m, 8H), 3.88-3.73 (m, 2H), 3.54 (q, J=7.7 Hz, 1H), 3.04 (d, J=41.0 Hz, 3H), 2.38-2.11 (m, 3H), 2.03-1.52 (m, 15H), 1.50-1.13 (m, 53H), 0.89 (t, J=6.7 Hz, 12H).

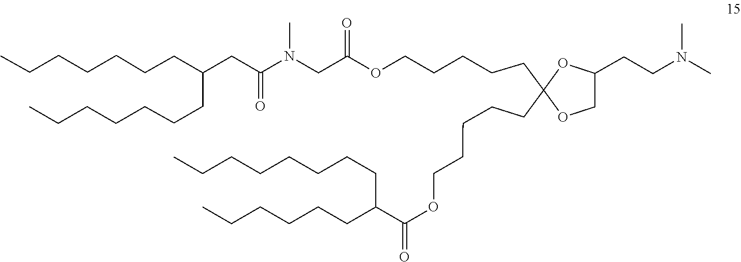 [SMILES: CCCCCCCCC(CCCCCC)C(=O)OCCCCCC1(CCCCCOC(=O)CN(C)C(=O)CC(CCCCCCC)CCCCCCC)OCC(CCN(C)C)O1]

(iv) 5-(4-(2-(Dimethylamino)ethyl)-2-(5-((N-(3-heptyldecanoyl)-N-methylglycyl)oxy)pentyl)-1,3-dioxolan-2-yl)pentyl 2-hexyldecanoate (15). ¹H NMR (400 MHz, CDCl₃, rotamers) δ 4.20-3.98 (m, 8H), 3.52-3.43 (m, 1H), 3.02 (d, J=40.5 Hz, 3H), 2.50-2.09 (m, 12H), 1.98-1.47 (m, 14H), 1.47-1.11 (m, 52H), 0.87 (br t, J=6.7 Hz, 12H).

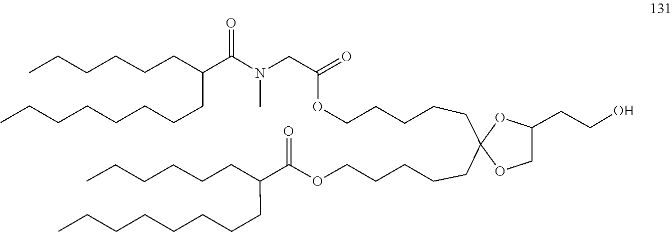 [SMILES: CCCCCCCCC(CCCCCC)C(=O)OCCCCCC1(CCCCCOC(=O)CN(C)C(=O)C(CCCCCC)CCCCCCCC)OCC(CCO)O1]

(v) 5-(2-(5-((N-(2-Hexyldecanoyl)-N-methylglycyl)oxy)pentyl)-4-(2-hydroxyethyl)-1,3-dioxolan-2-yl)pentyl 2-hexyldecanoate (131). From ketone 117. ¹H NMR (400 MHz, CDCl₃, rotamers) δ4.12-4.27 (m, 8H), 3.72-3.88 (m, 2H), 3.50-3.52 (m, 1H), 3.02 & 3.13 (s, 3H, rotamers 1:3), 2.29-2.34 (m, 3H), 1.89-2.06 (m, 2H), 1.81-1.87 (m, 2H), 1.57-1.64 (m, 10H), 1.26-1.46 (m, 54H), 0.89-1.00 (m, 12H).

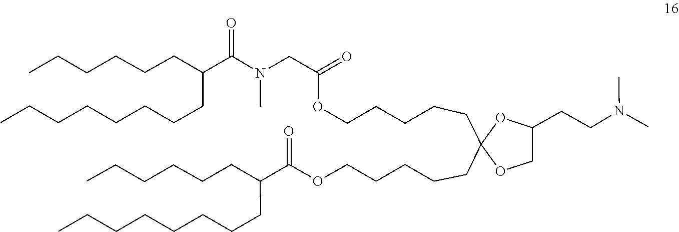 [SMILES: CCCCCCCCC(CCCCCC)C(=O)OCCCCCC1(CCCCCOC(=O)CN(C)C(=O)C(CCCCCC)CCCCCCCC)OCC(CCN(C)C)O1]

(vi) 5-(4-(2-(Dimethylamino)ethyl)-2-(5-((N-(2-hexyldecanoyl)-N-methylglycyl)oxy)pentyl)-1,3-dioxolan-2-yl)pentyl 2-hexyldecanoate (16). ¹H NMR (400 MHz, CDCl₃, rotamers) δ4.27-4.05 (m, 10H), 3.52-3.49 (m, 1H), 3.12 & 3.02 (s, 3H, rotamers, 3:1), 2.72-2.66 (m, 1H), 2.38-2.24 (m, 11H), 1.99-1.69 (m, 4H), 1.64-1.54 (m, 13H), 1.46-1.26 (m, 51H), 0.90-0.84 (br t, 12H).

(c) Procedure for the Conversion of a Ketone into a Lipid with a Type 3 Head Group.

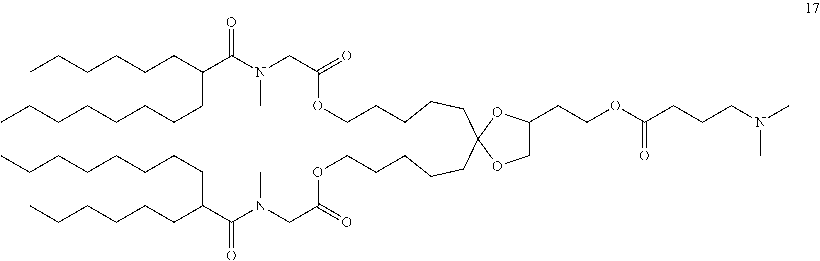 [SMILES: CCCCCCCCC(CCCCCC)C(=O)N(C)CC(=O)OCCCCCC1(CCCCCOC(=O)CN(C)C(=O)C(CCCCCC)CCCCCCCC)OCC(CCOC(=O)CCCN(C)C)O1]

(i) (4-(2-((4-(dimethylamino)butanoyl)oxy)ethyl)-1,3-dioxolane-2,2-diyl)bis(pentane-5,1-diyl) bis(2-(2-hexyl-N-methyldecanamido)acetate) (17). Obtained by esterification of hydroxyketal 129 with 4-(dimethylamino)butanoic acid hydrochloride according to procedure (a)-(i) above. ¹H NMR (400 MHz, CDCl₃, rotamers) δ 4.25-4.02 (m, 12H), 3.52-3.49 (m, 1H), 3.13 & 3.02 (s, 6H, rotamers 3:1) 2.72-2.66 (m, 2H), 2.43-2.24 (m, 10H), 1.99-1.18 (m, 68H), 0.90-0.87 (br t, 12H).

The following compound was obtained by the same method:

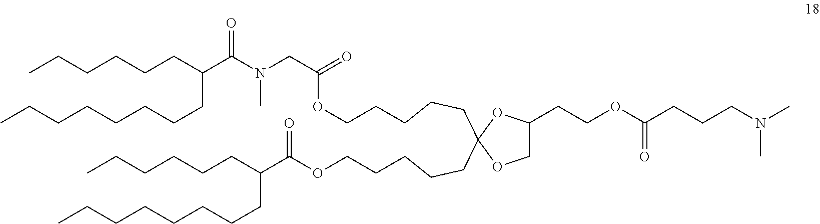 [SMILES: CCCCCCCCC(CCCCCC)C(=O)OCCCCCC1(CCCCCOC(=O)CN(C)C(=O)C(CCCCCC)CCCCCCCC)OCC(CCOC(=O)CCCN(C)C)O1]

(ii) 5-(4-(2-((4-(Dimethylamino)butanoyl)oxy)ethyl)-2-(5-((N-(2-hexyldecanoyl)-N-methyl-glycyl)oxy)pentyl)-1,3-dioxolan-2-yl)pentyl 2-hexyldecanoate (18). Obtained by esterification of hydroxyketal 131 with 4-(dimethylamino)butanoic acid. ¹H NMR (400 MHz, CDCl₃, rotamers) δ 4.28-4.05 (m, 10H), 3.52-3.49 (m, 1H), 3.13 & 3.02 (s, 3H, rotamers 3:1) 2.72-2.66 (m, 1H), 2.38-2.24 (m, 11H), 1.99-1.18 (m, 68H), 0.90-0.87 (br t, 12H).

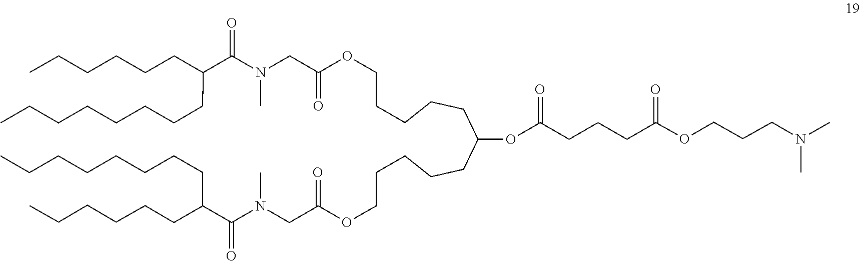 [SMILES: CCCCCCCCC(CCCCCC)C(=O)N(C)CC(=O)OCCCCCC(CCCCCOC(=O)CN(C)C(=O)C(CCCCCC)CCCCCCCC)OC(=O)CCCC(=O)OCCCN(C)C]

(d) Procedure for the conversion of a ketone into a lipid with a type 9 head group: 9,31-dihexyl-11,29-dimethyl-10,13,27,30-tetraoxo-14,26-dioxa-11,29-diazanonatriacon-tan-20-yl (3-(dimethylamino)propyl) glutarate (19). A solution of alcohol 121 from procedure (a)-(ii) above (435 mg, 0.52 mmol), glutaric anhydride (71 mg, 0.62 mmol) in pyridine (10 mL) was heated to 90 C for 18 hours under N₂. The reaction was cooled, diluted with EtOAc (15 mL) and washed with 2N HCl (15 mL), water (25 mL) and brine (25 mL). The organic phase was dried (Na₂SO₄) the volatiles were removed under vacuum and the residue of acid 132 (quantitative) was thoroughly dried under high vacuum and used as such without purification. To a solution of 132 (210 mg, 0.24 mmol), 3-(dimethylamino)propan-1-ol (24 mg, 0.24 mmol, 1 equiv) and 4-dimethylaminopyridine (15 mg, 0.12 mmol, 0.5 equiv) in CH₂Cl₂ (2 mL) was added 1-ethyl-3-(3-dimethylaminopropyl) carbodiimide (69 mg, 0.36 mmol, 1.5 equiv). The mixture was stirred for 16 h at rt under nitrogen, whereupon TLC indicated completion. The solution was diluted with ice water (10 mL) and extracted with CH₂Cl₂ (3×15 mL). The combined extracts were dried (Na₂SO₄), filtered, and concentrated under reduced pressure. The residue was purified by chromatography on silica gel (4-7% MeOH in DCM) to give 19 (138 mg, 61%) as a colorless gummy oil. ¹H NMR (400 MHz, CDCl₃, rotamers) δ4.90-4.84 (m, 1H), 4.16-4.10 (m, 10H), 3.13 & 3.02 (s, 3H, rotamers, 3:1), 2.73-2.66 (m, 2H), 2.42-2.35 (m, 6H), 2.28 (s, 6H), 1.99-1.92 (m, 2H), 1.88-1.81 (m, 2H), 1.67-1.27 (m, 67H), 0.90-0.87 (m, 12H).

(e) Procedure for the Conversion of a Ketone into a Lipid with a Type 8 Head Group.

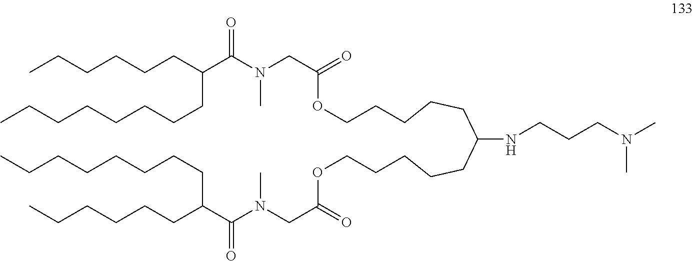 [SMILES: CCCCCCCCC(CCCCCC)C(=O)N(C)CC(=O)OCCCCCC(CCCCCOC(=O)CN(C)C(=O)C(CCCCCC)CCCCCCCC)NCCCN(C)C]

(i) 6-((3-(Dimethylamino)propyl)amino)undecane-1,11-diyl bis(2-(2-hexyl-N-methyl-decanamido)acetate) (133). To a solution of ketone 115 (821 mg, 1 mmol, 1 equiv), N¹,N¹-dimethyl-1,3-diaminopropane (207 mg, 2 mmol, 2 equiv), and acetic acid (1 drop) in 1,2-dichloroethane (20 mL) was added sodium triacetoxyborohydride (320 mg, 1.5 mmol, 1.5 equiv). The mixture was stirred at rt for 16 h, whereupon the reaction was complete (TLC). The solution was diluted with CH₂Cl₂ (50 mL) and washed with aq. saturated sodium bicarbonate solution (50 mL). The solution was dried (Na₂SO₄) and concentrated. The colorless residual oil was purified by chromatography on silica gel (40-50% EtOAc in hexane) to give 133 as a colorless gummy oil (quant.). ¹H NMR (400 MHz, CDCl₃, rotamers) δ4.23-4.05 (m, 8H), 3.18-2.91 (m, 11H, rotamers), 2.82-2.68 (m, 8H), 2.33-2.25 (m, 2H), 1.70-1.27 (m, 64H) 0.90-0.87 (br t, 12H).

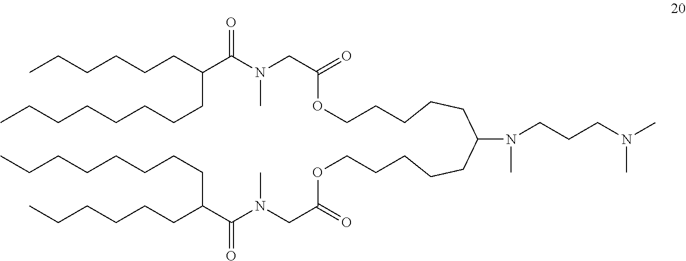 [SMILES: CCCCCCCCC(CCCCCC)C(=O)N(C)CC(=O)OCCCCCC(CCCCCOC(=O)CN(C)C(=O)C(CCCCCC)CCCCCCCC)N(C)CCCN(C)C]

(ii) 6-((3-(Dimethylamino)propyl)(methyl)amino)undecane-1,11-diyl bis(2-(2-hexyl-N-methyldecanamido)acetate) (20). A mixture of 133 (323 mg, 0.357 mmol), aq. formaldehyde (37%, 1 mL) and NaBH(OAc)₃ (379 mg, 1.8 mmol) in THF (8 mL) was stirred under inert atmosphere at room temperature for 3 days. The reaction was then quenched with sat. aq. NaHCO₃ (3 mL), diluted with water (5 mL) and extracted with DCM (3×10 mL). The combined extracts were dried (Na₂SO₄) and concentrated. The residue was purified by silica chromatography (0-5% MeOH in DCM) to yield lipid 20 (360 mg, 94%) as an oil. ¹H NMR (400 MHz, CDCl₃, rotamers) δ 4.15-4.11 (m, 8H), 3.13, 3.02 (s, 6H, rotamer, 1:3), 2.96-2.92 (m, 2H), 2.73-2.67 (m, 8H), 2.49-2.40 (m, 2H), 2.36-2.30 (m, 1H), 2.14 (s, 3H), 1.82-1.49 (m, 16H), 1.42-1.05 (m, 60H) 0.90-0.84 (br t, 12H).

The following compounds were prepared by the same method:

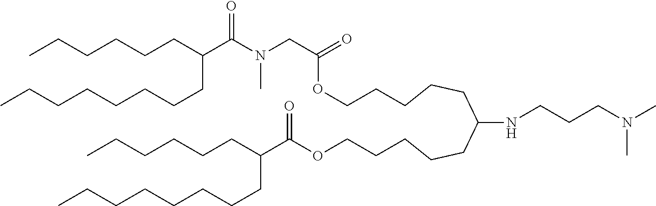 [SMILES: CCCCCCCCC(CCCCCC)C(=O)OCCCCCC(CCCCCOC(=O)CN(C)C(=O)C(CCCCCC)CCCCCCCC)NCCCN(C)C]

(iii) 6-((3-(Dimethylamino)propyl)amino)-11-((N-(2-hexyldecanoyl)-N-methylglycyl)oxy)-undecyl 2-hexyldecanoate. From ketone 117. ¹H NMR (400 MHz, CDCl₃, rotamers) δ4.17-4.06 (m, 6H), 3.18-2.78 (m, 6H, rotamers), 2.77-2.67 (m, 3H), 2.44 (s, 6H), 2.36-2.29 (m, 1H), 2.06-2.01 (m, 1H), 1.68-1.26 (m, 66H), 0.90-0.87 (br t, 12H).

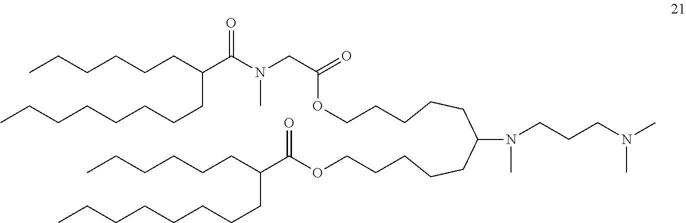 [SMILES: CCCCCCCCC(CCCCCC)C(=O)OCCCCCC(CCCCCOC(=O)CN(C)C(=O)C(CCCCCC)CCCCCCCC)N(C)CCCN(C)C]

(iv) 6-((3-(Dimethylamino)propyl)(methyl)amino)-11-((N-(2-hexyldecanoyl)-N-methylglyc-yl)oxy)undecyl 2-hexyldecanoate (21). ¹H NMR (400 MHz, CDCl₃, rotamers) δ 4.21-4.03 (m, 6H), 3.16-2.99 (m, 8H, rotamers), 2.74-2.68 (m, 7H), 2.35-2.23 (m, 3H), 1.72-1.26 (m, 67H), 0.90-0.86 (m, 12H).

(e) Procedure for the Conversion of a Ketone into a Lipid with a Type 7 Head Group.

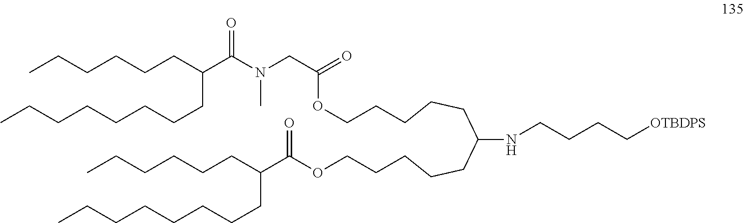

(i) 6-((4-((tert-Butyldiphenylsilyl)oxy)butyl)amino)-11-((N-(2-hexyldecanoyl)-N-methyl-glycyl)oxy)undecyl 2-hexyldecanoate (135). To a solution of ketone 117 (750 mg, 1 mmol, 1 equiv), 4-((tert-butyldiphenylsilyl)oxy)butan-1-amine, 134 (656 mg, 2 mmol, 2 equiv), and acetic acid (1 drop) in 1,2-dichloroethane (20 mL) was added sodium triacetoxyborohydride (320 mg, 1.5 mmol, 1.5 equiv). The mixture was stirred at rt for 16 h, whereupon the reaction was complete (TLC). The solution was diluted with CH₂Cl₂ (50 mL) and washed with aq. saturated sodium bicarbonate solution (50 mL). The solution was dried (Na₂SO₄) and concentrated. The colorless residual oil was purified by chromatography on silica gel (40-50% EtOAc in hexane) to give 135 as a colorless gummy oil (quant.). ¹H NMR (400 MHz, CDCl₃, rotamers) δ7.69-7.66 (m, 4H), 7.43-7.39 (m, 6H), 4.15-4.06 (m, 6H), 3.70-3.67 (m, 2H), 3.13 & 3.02 (s, 3H, rotamers 1:3), 2.73-2.29 (m, 6H), 1.65-1.57 (m, 10H), 1.46-1.27 (m, 52H), 1.06 (s, 9H), 0.84-0.90 (br t, 12H).

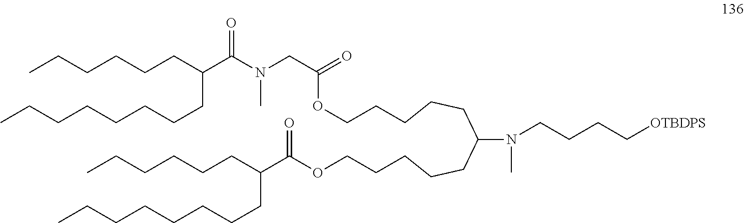

(ii) 6-((4-((tert-butyldiphenylsilyl)oxy)butyl)(methyl)amino)-11-((N-(2-hexyldecanoyl)-N-methylglycyl)oxy)undecyl 2-hexyldecanoate (136). A mixture of 135 (378 mg, 0.357 mmol), aq. formaldehyde (37%, 1 mL) and NaBH(OAc)₃ (379 mg, 1.8 mmol) in THF (8 mL) was stirred under inert atmosphere at room temperature for 3 days. The reaction was then quenched with sat. aq. NaHCO₃ (3 mL), diluted with water (5 mL) and extracted with DCM (3×10 mL). The combined extracts were dried (Na₂SO₄) and concentrated. The residue was purified by silica chromatography (0-5% MeOH in DCM) to yield the tertiary amine 136 (360 mg, 94%) as an oil. ¹H NMR (400 MHz, CDCl₃, rotamers) δ7.69-7.61 (m, 4H), 7.44-7.39 (m, 6H), 4.15-4.04 (m, 6H), 3.69-3.66 (m, 2H), 3.12 & 3.02 (s, 3H, rotamers 1:3), 2.72-2.67 (m, 1H), 2.44-2.30 (m, 4H), 2.14 (s, 3H), 1.77 (br s, 2H), 1.76-1.60 (m, 10H), 1.46-1.26 (m, 57H), 1.06 (s, 9H), 0.92-0.85 (m, 12H).

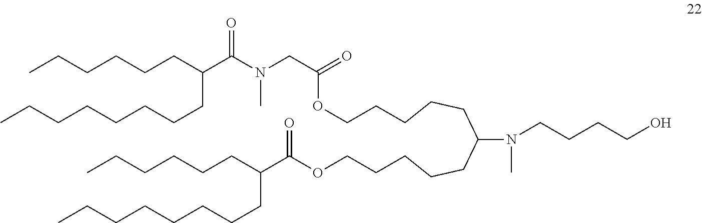 [SMILES: CCCCCCCCC(CCCCCC)C(=O)OCCCCCC(CCCCCOC(=O)CN(C)C(=O)C(CCCCCC)CCCCCCCC)N(C)CCCCO]

(iii) 11-((N-(2-hexyldecanoyl)-N-methylglycyl)oxy)-6-((4-hydroxybutyl)(methyl)amino)-undecyl 2-hexyl-decanoate (22). To a solution of 136 (360 mg, 0.335 mmol) in THF (3 mL) was added HF-pyridine (0.4 mL) at 0° C. under nitrogen atmosphere. The reaction was warmed to room temperature and stirred for 18 hours. Water (7 mL) was added, and the mixture was extracted with DCM (3×7 mL). The combined extracts were dried (Na₂SO₄) and concentrated. The residue was purified by silica chromatography (0-5% MeOH in DCM) to yield lipid 22 (196 mg, 70%) as an oil. ¹H NMR (400 MHz, CDCl₃, rotamers) δ4.31-4.06 (m, 6H), 3.81-3.63 (m, 2H), 3.21 (br s, 2H), 3.15 & 3.02 (s, 3H, rotamers, 3:1), 2.82-2.79 (m, 2H), 2.74-2.67 (m, 1H), 2.01-1.86 (m, 3H), 1.78-1.44 (m, 26H), 1.38-1.27 (m, 43H), 0.91-0.85 (br t, 12H).

The following compounds were prepared by the same method:

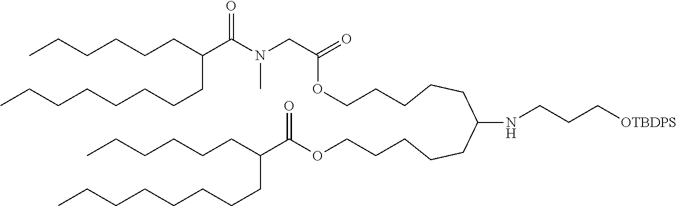

(iv) 6-((3-((tert-Butyldiphenylsilyl)oxy)propyl)amino)-11-((N-(2-hexyldecanoyl)-N-methyl-glycyl)oxy)undecyl 2-hexyldecanoate. Prepared from ketone 117 and 3-((tert-butyldiphenylsilyl)oxy)propan-1-amine. ¹H NMR (400 MHz, CDCl₃, rotamers) δ7.69-7.66 (m, 4H), 7.44-7.39 (m, 6H), 4.14-4.05 (m, 6H), 3.77-3.74 (m, 2H), 3.12 & 3.02 (s, 3H, rotamers 1:3), 2.75-2.30 (m, 5H), 1.77 (br s, 2H), 1.65-1.57 (m, 10H), 1.46-1.26 (m, 55H), 1.07 (s, 9H), 0.90-0.84 (m, 12H).

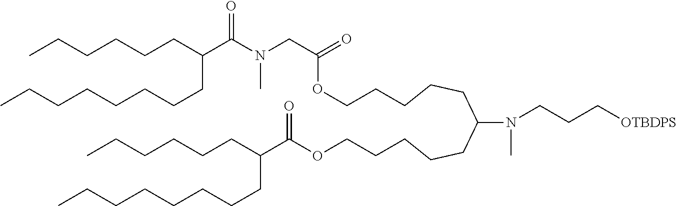

(v) 6-((3-((tert-Butyldiphenylsilyl)oxy)propyl)(methyl)amino)-11-((N-(2-hexyldecanoyl)-N-methylglycyl)oxy)undecyl 2-hexyldecanoate. ¹H NMR (400 MHz, CDCl₃, rotamers) δ7.69-7.61 (m, 4H), 7.44-7.39 (m, 6H), 4.15-4.04 (m, 6H), 3.79-3.71 (m, 2H), 3.12 & 3.02 (s, 3H, rotamers 1:3), 2.72-2.67 (m, 1H), 2.42-2.29 (m, 4H), 2.13 (s, 3H), 1.77 (br s, 2H), 1.76-1.60 (m, 10H), 1.46-1.26 (m, 54H), 1.07 (s, 9H), 0.90-0.84 (m, 12H).

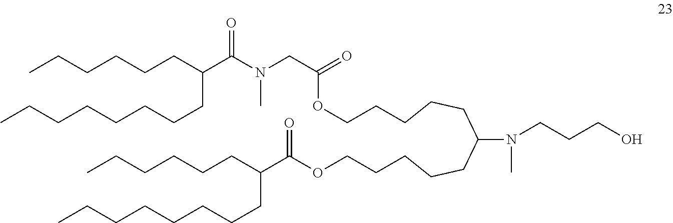 [SMILES: CCCCCCCCC(CCCCCC)C(=O)OCCCCCC(CCCCCOC(=O)CN(C)C(=O)C(CCCCCC)CCCCCCCC)N(C)CCCO]

(vi) 11-((N-(2-Hexyldecanoyl)-N-methylglycyl)oxy)-6-((3-hydroxypropyl)(methyl)amino)-undecyl 2-hexyldecanoate (23). ¹H NMR (400 MHz, CDCl₃, rotamers) δ4.18-4.00 (m, 8H), 3.41-3.32 (m, 3H), 3.16 & 3.02 (s, 3H, rotamers 1:3), 2.86-2.81 (dd, 3H), 2.73-2.69 (m, 1H), 2.34-2.30 (m, 1H), 2.07-1.86 (m, 2H), 1.76-1.44 (m, 26H), 1.37-1.27 (m, 38H), 0.90-0.84 (br t, 12H).

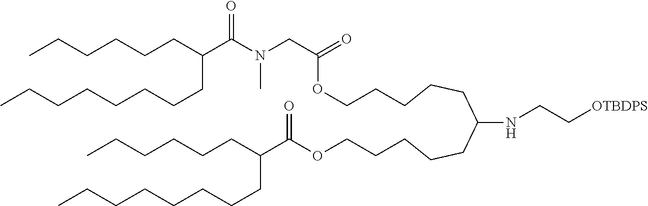

(vii) 6-((2-((tert-Butyldiphenylsilyl)oxy)ethyl)amino)-11-((N-(2-hexyldecanoyl)-N-methyl-glycyl)oxy)undecyl 2-hexyldecanoate. Prepared from ketone 117 and 2-((tert-butyldiphenylsilyl)oxy)ethan-1-amine. ¹H NMR (400 MHz, CDCl₃, rotamers) δ7.69-7.67 (m, 4H), 7.45-7.39 (m, 6H), 4.15-4.06 (m, 6H), 3.70-3.67 (m, 2H), 3.12 & 3.02 (s, 3H, rotamers 1:3), 2.73-2.66 (m, 2H), 2.53-2.28 (m, 2H) 1.70-1.52 (m, 10H), 1.46-1.26 (m, 56H), 1.07 (s, 9H), 0.90-0.84 (m, 12H).

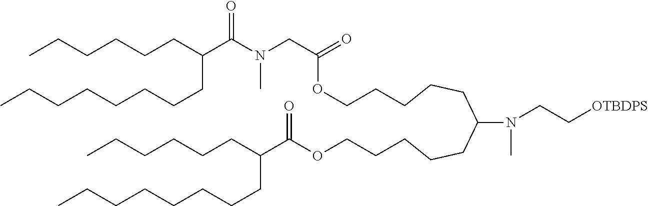

(viii) 6-((2-((tert-Butyldiphenylsilyl)oxy)ethyl)(methyl)amino)-11-((N-(2-hexyldecanoyl)-N-methylglycyl)oxy)undecyl 2-hexyldecanoate. ¹H NMR (400 MHz, CDCl₃, rotamers) δ7.71-7.65 (m, 4H), 7.47-7.37 (m, 6H), 4.15-4.04 (m, 6H), 3.69-3.66 (m, 2H), 3.12 & 3.02 (s, 3H, rotamers 1:3), 2.73-2.26 (m, 5H), 2.16 (s, 3H) 1.67-1.52 (m, 9H), 1.46-1.27 (m, 54H), 1.07 (s, 10H), 0.90-0.84 (m, 12H).

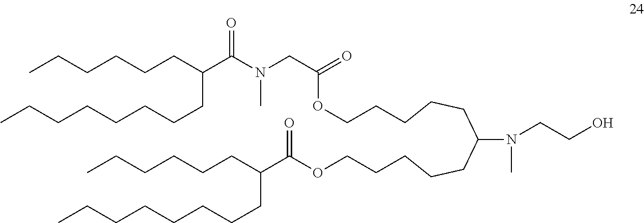 [SMILES: CCCCCCCCC(CCCCCC)C(=O)OCCCCCC(CCCCCOC(=O)CN(C)C(=O)C(CCCCCC)CCCCCCCC)N(C)CCO]

(ix) 11-((N-(2-Hexyldecanoyl)-N-methylglycyl)oxy)-6-((2-hydroxyethyl)(methyl)amino)-undecyl 2-hexyldecanoate (24). ¹H NMR (400 MHz, CDCl₃, rotamers) δ4.20-3.92 (m, 8H), 3.44-3.18 (m, 3H), 3.15 & 3.02 (s, 3H, rotamers 1:3), 2.94-2.90 (dd, 3H), 2.80-2.67 (m, 1H), 2.36-2.29 (m, 1H), 1.80-1.44 (m, 26H), 1.31-1.27 (m, 38H), 0.90-0.84 (m, 12H).

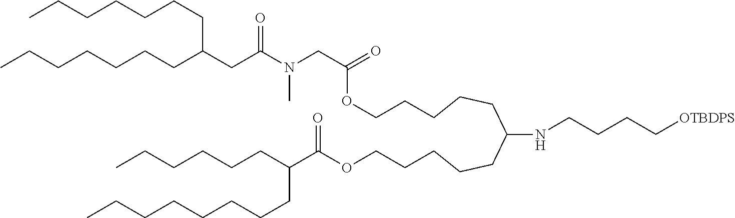

(x) 6-((4-((tert-Butyldiphenylsilyl)oxy)butyl)amino)-11-((N-(3-heptyldecanoyl)-N-methyl-glycyl)oxy)undecyl 2-hexyldecanoate. From ketone 123 and amine 134. ¹H NMR (400 MHz, CDCl₃, rotamers) δ 7.68-7.64 (m, 4H), 7.44-7.34 (m, 6H), 4.18-4.00 (m, 6H), 3.66 (tr, J=6 Hz, 2H), 3.07 & 2.97 (s, 3H, rotamer), 2.62-2.35 (m, 3H), 2.32-2.10 (m, 3H), 1.93 (m, 1H), 1.66-1.55 (m, 10H), 1.33-1.21 (m, 59H), 1.04 (s, 9H), 0.90-0.85 (br t, 12H).

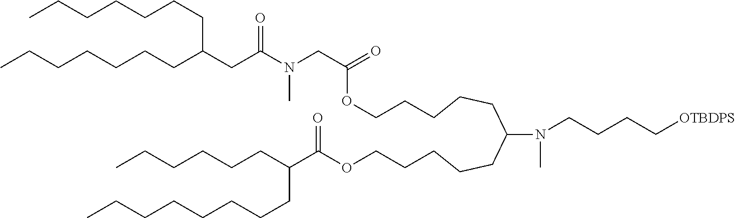

(xi) 6-((4-((tert-Butyldiphenylsilyl)oxy)butyl)(methyl)amino)-11-((N-(3-heptyldecanoyl)-N-methylglycyl)oxy)undecyl 2-hexyldecanoate. ¹H NMR (400 MHz, CDCl₃, rotamers) δ7.68-7.64 (m, 4H), 7.44-7.34 (m, 6H), 4.19-3.99 (m, 6H), 3.66 (tr, 2H, J=6.2 Hz), 3.06 & 2.96 (s, 3H, rotamers), 2.44-2.26 (m, 6H), 2.12 (s, 3H), 1.93 (m, 1H), 1.66-1.55 (m, 9H) 1.31-1.23 (m, 59H), 1.04 (s, 9H), 0.91-0.81 (br t, 12H).

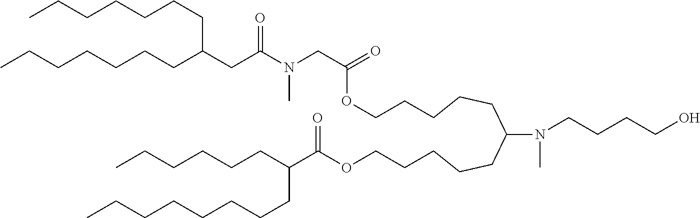 [SMILES: CCCCCCCCC(CCCCCC)C(=O)OCCCCCC(CCCCCOC(=O)CN(C)C(=O)CC(CCCCCCC)CCCCCCC)N(C)CCCCO]

(xi) 11-((N-(3-Heptyldecanoyl)-N-methylglycyl)oxy)-6-((4-hydroxybutyl)(methyl)amino)-undecyl 2-hexyldecanoate (25). ¹H NMR (400 MHz, CDCl₃) including rotamers: δ 4.20-4.02 (m, 6H), 3.71 (m, 2H), 3.22-3.10 (m, 3H), 3.09 and 2.96 (s, 3H, rotamers), 2.78 (s, 3H), 2.31-2.10 (m, 3H), 1.46-1.39 (m, 10H), 1.33-1.22 (m, 70H), 0.89-0.85 (tr, 12H).

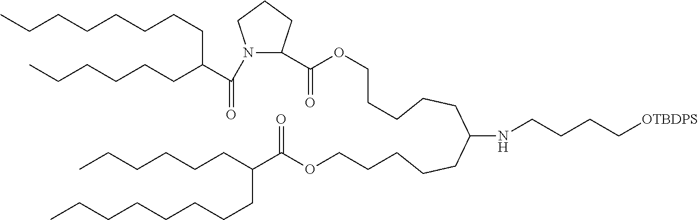

(xii) 6-((4-((tert-Butyldiphenylsilyl)oxy)butyl)amino)-11-((2-hexyldecanoyl)oxy)undecyl (2-hexyldecanoyl)prolinate. From ketone 137 and amine 134. ¹H NMR (400 MHz, CDCl₃, rotamers) δ 7.68 (dt, J=6.5, 1.7 Hz, 4H), 7.39-7.41 (m, 6H), 4.51 (dt, J=8.4, 4.2 Hz, 1H), 4.18-3.97 (m, 4H), 3.76-3.52 (m, 4H), 2.75-1.83 (m, 10H), 1.78-1.13 (m, 67H), 1.06 (s, 9H), 0.90-0.86 (br t, 12H).

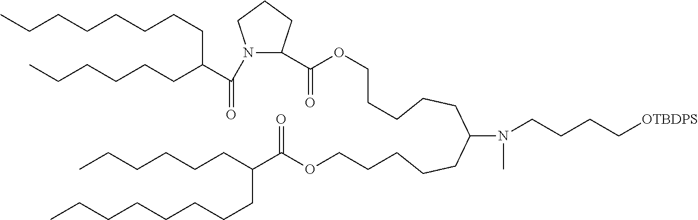

(xiii) 6-((4-((tert-Butyldiphenylsilyl)oxy)butyl)(methyl)amino)-11-((2-hexyldecanoyl)oxy)-undecyl (2-hexyldecanoyl)prolinate. ¹H NMR (400 MHz, CDCl₃, rotamers) δ7.69-7.67 (m, 4H), 7.45-7.37 (m, 6H), 4.49-4.48 (m, 1H), 4.82-4.76 (m, 1H), 4.53-4.46 (m, 1H), 4.18-4.05 (m, 4H), 3.76-3.54 (m, 4H), 2.53-2.29 (m, 4H), 2.24-1.95 (m, 6H), 1.75-1.57 (m, 12H), 1.64-1.27 (m, 56H), 1.06 (s, 9H), 0.90-0.84 (br t, 12H).

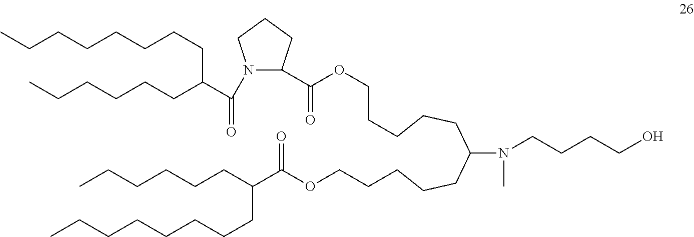 [SMILES: CCCCCCCCC(CCCCCC)C(=O)OCCCCCC(CCCCCOC(=O)C1CCCN1C(=O)C(CCCCCC)CCCCCCCC)N(C)CCCCO]

(xiv) 11-((2-Hexyldecanoyl)oxy)-6-((4-hydroxybutyl)(methyl)amino)undecyl (2-hexyl-decanoyl)prolinate (26). ¹H NMR (400 MHz, CDCl₃, rotamers) δ 4.47 (dt, J=7.7, 3.7 Hz, 1H), 4.27-4.00 (m, 4H), 3.84-3.51 (m, 4H), 3.19 (m, 3H), 2.81 (dd, J=11.7, 4.9 Hz, 3H), 2.40-1.04 (m, 72H), 0.89 (br t, 12H).

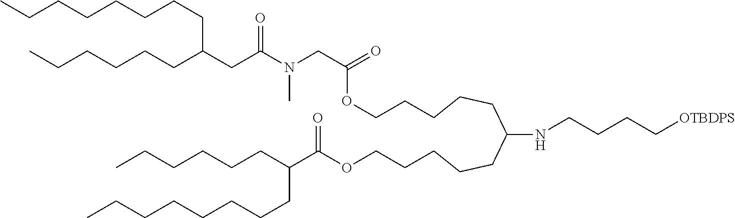

(xv) 6-((4-((tert-Butyldiphenylsilyl)oxy)butyl)amino)-11-((N-(3-hexylundecanoyl)-N-methyl-glycyl)oxy)undecyl 2-hexyldecanoate. From ketone 125 and amine 134. ¹H NMR (400 MHz, CDCl₃, rotamers) δ 7.71-7.62 (m, 4H), 7.45-7.34 (m, 6H), 4.17-4.00 (m, 6H), 3.66 (m, 2H), 3.07 and 2.97 (s, 3H, rotamer), 2.62-2.35 (m, 4H), 2.32-2.10 (m, 3H), 1.92 (m, 1H), 1.72-1.50 (m, 10H), 1.46-1.20 (m, 58H), 1.04 (s, 9H), 0.92-0.82 (br t, 12H).

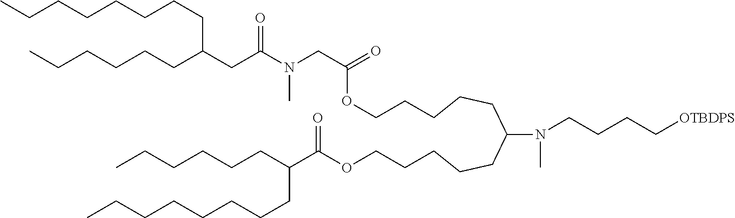

(xvi) 6-((4-((tert-Butyldiphenylsilyl)oxy)butyl)(methyl)amino)-11-((N-(3-hexylundecanoyl)-N-methylglycyl)oxy)undecyl 2-hexyldecanoate. ¹H NMR (400 MHz, CDCl₃, rotamers) δ 7.71-7.62 (m, 4H), 7.46-7.33 (m, 6H), 4.21-3.99 (m, 6H), 3.66 (t, J=6 Hz, 2H), 3.06 & 2.96 (s, 3H, rotamers), 2.44-2.24 (m, 6H), 2.13 (s, 3H), 1.92 (m, 1H), 1.67-1.52 (m, 10H), 1.35-1.20 (m, 58H), 1.04 (s, 9H), 0.92-0.84 (br t, 12H).

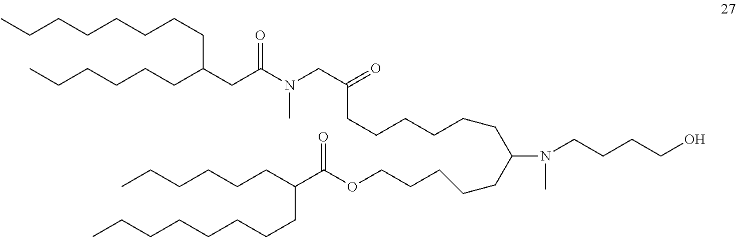 [SMILES: CCCCCCCCC(CCCCCC)CC(=O)N(C)CC(=O)CCCCCCC(CCCCCOC(=O)C(CCCCCC)CCCCCCCC)N(C)CCCCO]

(xvii) 11-((N-(3-Hexylundecanoyl)-N-methylglycyl)oxy)-6-((4-hydroxybutyl)(methyl)-amino)-undecyl 2-hexyldecanoate (27). ¹H NMR (400 MHz, CDCl₃, rotamers) δ4.18-4.02 (m, 6H), 3.76-3.57 (m, 2H), 3.26-2.94 (m, 6H), 2.85 & 2.78 (s, 3H, rotamer), 2.33-2.09 (m, 3H), 1.99-1.50 (m, 16H), 1.51-1.36 (m, 10H), 1.35-1.16 (m, 43H), 0.92-0.82 (br t, 12H).

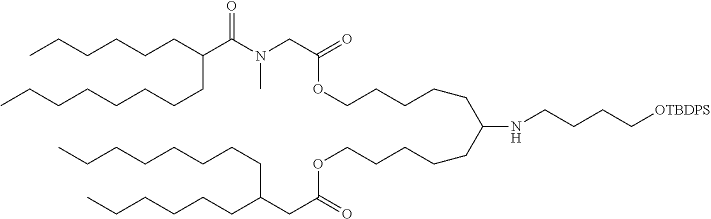

(xviii) 6-((4-((tert-Butyldiphenylsilyl)oxy)butyl)amino)-11-((N-(2-hexyldecanoyl)-N-methyl-glycyl)oxy)undecyl 3-hexylundecanoate. From ketone 138 and amine 134. ¹H NMR (400 MHz, CDCl₃, rotamers) δ7.71-7.60 (m, 4H), 7.45-7.33 (m, 6H), 4.17-4.00 (m, 6H), 3.66 (tr, J=6 Hz, 2H), 3.10 & 3.00 (s, 3H, rotamer), 2.68 (m, 1H), 2.56 (br, 1H), 2.40 (tr, J=7.3, 2H), 2.26-2.18 (m, 3H), 1.69-1.90 (m, 69H), 1.04 (s, 9H), 0.89-0.85 (br t, 12H).

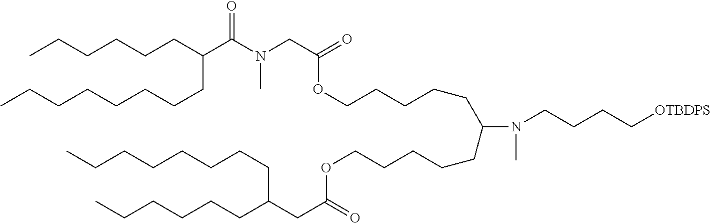

(xix) 6-((4-((tert-Butyldiphenylsilyl)oxy)butyl)(methyl)amino)-11-((N-(2-hexyldecanoyl)-N-methylglycyl)oxy)undecyl 3-hexylundecanoate. ¹H NMR (400 MHz, CDCl₃, rotamers) δ7.71-7.61 (m, 4H), 7.46-7.32 (m, 6H), 4.16-4.00 (m, 6H), 3.66 (tr, J=6.4 Hz, 2H), 3.10 & 3.00 (s, 3H, rotamer), 2.68 (m, 1H), 2.40 (tr, 2H), 2.24-2.19 (m, 3H), 2.12 (s, 3H), 1.87-1.14 (m, 67H), 1.04 (s, 9H), 0.89-0.85 (tr, 12H).

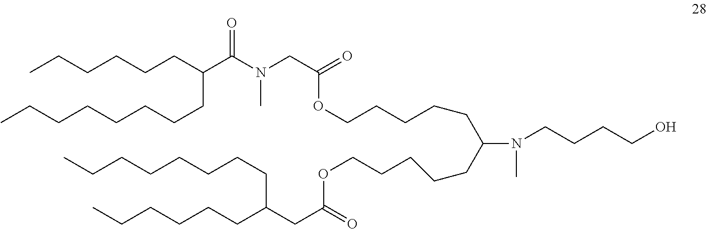 [SMILES: CCCCCCCCC(CCCCCC)CC(=O)OCCCCCC(CCCCCOC(=O)CN(C)C(=O)C(CCCCCC)CCCCCCCC)N(C)CCCCO]

(xx) 11-((N-(2-Hexyldecanoyl)-N-methylglycyl)oxy)-6-((4-hydroxybutyl)(methyl)amino)-undecyl 3-hexylundecanoate (28). ¹H NMR (400 MHz, CDCl₃, rotamers) δ4.18-4.00 (m, 6H), 3.77-3.57 (m, 2H), 3.26-2.97 (m, 6H), 2.88-2.74 (m, 3H), 2.69 (m, 1H), 2.25-2.17 (m, 2H), 2.00-1.21 (m, 70H), 0.9-0.83 (br t, 12H).

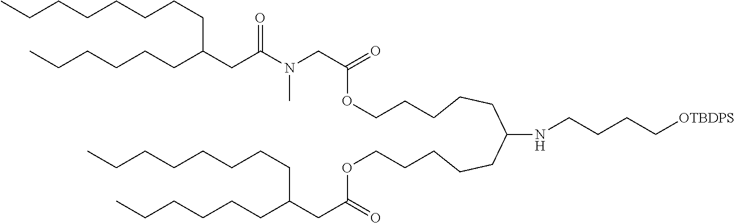

(xxi) 6-((4-((tert-Butyldiphenylsilyl)oxy)butyl)amino)-11-((N-(3-hexylundecanoyl)-N-methylglycyl)oxy)undecyl 3-hexylundecanoate. From ketone 123 and amine 134. ¹H NMR (400 MHz, CDCl₃, rotamers) δ7.71-7.61 (m, 4H), 7.45-7.33 (m, 6H), 4.15-4.00 (m, 6H), 3.72-3.62 (m, 2H), 3.07 & 2.97 (s, 3H), 2.32-2.10 (m, 4H), 1.94-1.79 (m, 2H), 1.75-1.45 (m, 22H), 1.36-1.21 (m, 50H), 1.04 (s, 9H), 0.89-0.84 (m, 12H).

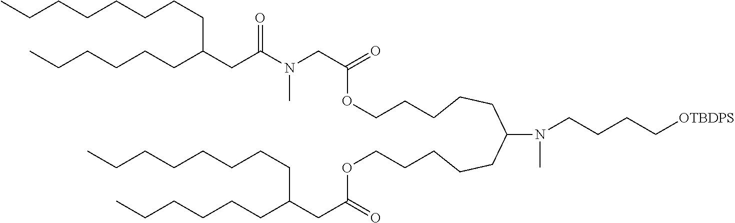

(xxii) 6-((4-((tert-Butyldiphenylsilyl)oxy)butyl)(methyl)amino)-11-((N-(3-hexylundecanoyl)-N-methylglycyl)oxy)undecyl 3-hexylundecanoate. ¹H NMR (400 MHz, CDCl₃, rotamers) δ 7.71-7.60 (m, 4H), 7.44-7.32 (m, 6H), 4.18-3.98 (m, 6H), 3.66 (tr, J=6 Hz, 2H), 3.06 & 2.97 (s, 3H), 2.65 (m, 1H), 2.38-2.17 (m, 6H), 2.12 (s, 3H), 1.92 (m, 1H), 1.84 (m, 1H), 1.66-1.53 (m, 16H), 1.42-1.18 (m, 52H), 1.04 (s, 9H), 0.91-0.85 (br t, 12H).

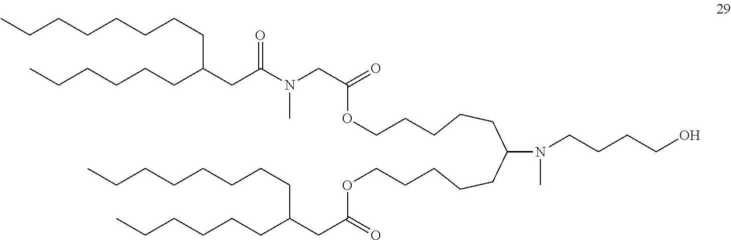 [SMILES: CCCCCCCCC(CCCCCC)CC(=O)OCCCCCC(CCCCCOC(=O)CN(C)C(=O)CC(CCCCCC)CCCCCCCC)N(C)CCCCO]

(xxiii) 11-((N-(3-Hexylundecanoyl)-N-methylglycyl)oxy)-6-((4-hydroxybutyl)(methyl)-amino)undecyl 3-hexylundecanoate (29). ¹H NMR (400 MHz, CDCl₃, rotamers) δ4.20-4.00 (m, 6H), 3.73 (m, 2H), 3.25 & 2.94 (m, 6H), 2.77 (s, 3H), 2.32-2.10 (m, 4H), 1.98-1.89 (m, 2H), 1.73-1.56 (m, 12H), 1.49-1.37 (m, 8), 1.33-1.20 (m, 48), 0.91-0.86 (br t, 12H)

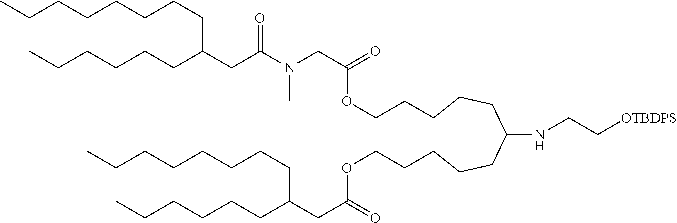

(xxiv) 6-((2-((tert-Butyldiphenylsilyl)oxy)ethyl)amino)-11-((N-(3-hexylundecanoyl)-N-methylglycyl)oxy)undecyl 3-hexylundecanoate. Prepared from ketone 123 and 2-((tert-butyldiphenylsilyl)oxy)ethan-1-amine. ¹H NMR (400 MHz, CDCl₃, rotamers) δ7.74-7.59 (m, 4H), 7.45-7.33 (m, 6H), 4.19-3.98 (m, 6H), 3.78 (tr, 3H), 3.06 & 2.97 (s, 3H), 2.69 (m, 2H), 2.48 (br, 1H), 2.31-2.09 (m, 4H), 1.92 (m, 1H), 1.82 (m, 1H), 1.67-1.55 (m, 6H), 1.42-1.21 (m, 56), 1.04 (s, 9H), 0.91-0.85 (br t, 12H).

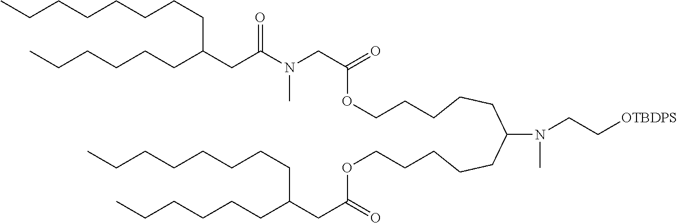

(xxv) 6-((2-((tert-Butyldiphenylsilyl)oxy)ethyl)(methyl)amino)-11-((N-(3-hexylundecanoyl)-N-methylglycyl)oxy)undecyl 3-hexylundecanoate. ¹H NMR (400 MHz, CDCl₃, rotamers) δ7.70-7.63 (m, 4H), 7.45-7.34 (m, 6H), 4.22-3.93 (m, 6H), 3.72 (t, 2H), 3.07 & 2.97 (s, 3H), 2.62 (t, 2H), 2.40 (m, 1H), 2.30-2.10 (m, 6H), 1.93 (m, 1H), 1.83 (m, 1H), 1.65-1.56 (m, 4H), 1.49-1.37 (m, 2H), 1.36-1.15 (m, 57H), 1.04 (s, 9H), 0.90-0.85 (br t, 12H).

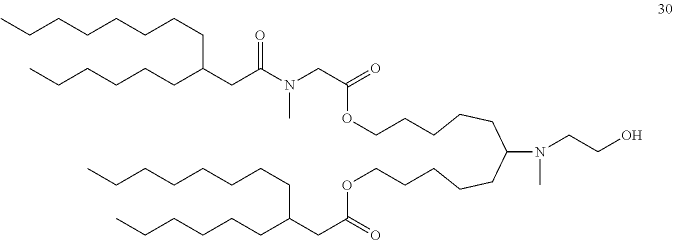 [SMILES: CCCCCCCCC(CCCCCC)CC(=O)OCCCCCC(CCCCCOC(=O)CN(C)C(=O)CC(CCCCCC)CCCCCCCC)N(C)CCO]

(xxvi) 11-((N-(3-Hexylundecanoyl)-N-methylglycyl)oxy)-6-((2-hydroxyethyl)(methyl)-amino)-undecyl 3-hexylundecanoate (30). ¹H NMR (400 MHz, CDCl₃, rotamers) δ4.18-3.98 (m, 6H), 3.40 (m, 2H), 3.28-2.86 (m, 9H), 2.31-2.09 (m, 4H), 1.90 (m, 1H), 1.82 (m, 1H), 1.75-1.48 (m, 8H), 1.46-1.17 (m, 57H), 0.90-0.82 (br t, 12H).

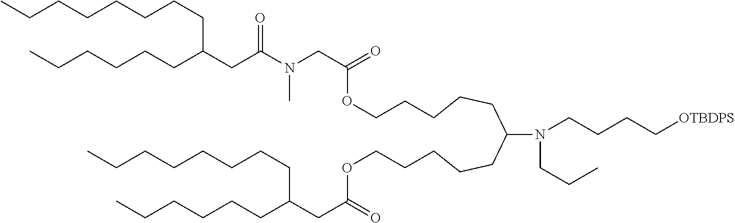

(xxvii) 6-((4-((tert-Butyldiphenylsilyl)oxy)butyl)(propyl)amino)-11-((N-(3-hexylundecanoyl)-N-methylglycyl)oxy)undecyl 3-hexylundecanoate. From 6-((2-((tert-butyldiphenylsilyl)-oxy)ethyl)amino)-11-((N-(3-hexylundecanoyl)-N-methylglycyl)oxy)undecyl 3-hexylundecanoate of part (xiii) above according to procedure (ii) above, but by the use of propionaldehyde instead of formaldehyde. ¹H NMR (400 MHz, CDCl₃, rotamers) δ7.71-7.62 (m, 4H), 7.44-7.34 (m, 6H), 4.19-3.98 (m, 6H), 3.65 (tr, 2H), 3.06 & 2.97 (s, 3H), 2.39-2.10 (m, 9H), 1.93 (m, 1H), 1.83 (m, 1H), 1.67-1.51 (m, 9H), 1.45-1.18 (m, 58H), 1.04 (s, 9H), 0.89-0.85 (br t, 12H)

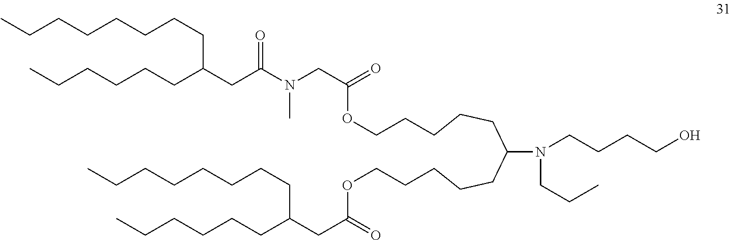 [SMILES: CCCCCCCCC(CCCCCC)CC(=O)OCCCCCC(CCCCCOC(=O)CN(C)C(=O)CC(CCCCCC)CCCCCCCC)N(CCC)CCCCO]

(xxiii) 11-((N-(3-Hexylundecanoyl)-N-methylglycyl)oxy)-6-((4-hydroxybutyl)(propyl)-amino)-undecyl 3-hexylundecanoate (31). ¹H NMR (400 MHz, CDCl₃, rotamers) δ4.41-4.00 (m, 6H), 3.72 (m, 2H), 3.27-2.88 (m, 8H), 2.34-2.10 (m, 4H), 2.02-1.53 (m, 18H), 1.48-1.37 (m, 31 8H), 1.33-1.17 (m, 46H), 1.09-0.99 (tr, J=7 Hz, 3H), 0.90-0.83 (br t, 12H).

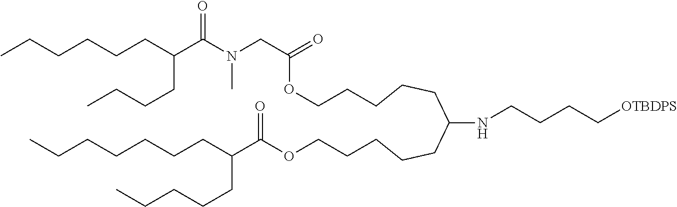

(xxix) 11-((N-(4-Butyldecanoyl)-N-methylglycyl)oxy)-6-((4-((tert-butyldiphenylsilyl)oxy)-butyl)-amino)undecyl 4-pentylundecanoate. From ketone 139 and amine 134. ¹H NMR (400 MHz, CDCl₃, rotamers) δ 7.71-7.62 (m, 4H), 7.46-7.34 (m, 6H), 4.18-4.00 (m, 6H), 3.67 (tr, J=6 Hz, 2H), 3.07 & 2.97 (s, 3H), 2.55 (tr, J=6 Hz, 2H), 2.45 (m, 1H), 2.37-2.17 (m, 4H), 1.90-1.45 (m, 16H), 1.37-1.21 (m, 44H), 1.04 (s, 9H), 0.91-0.85 (br t, 12H)

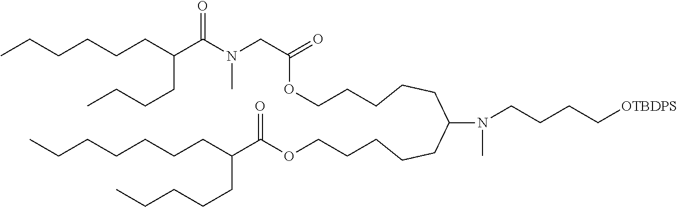

(xxx) 11-((N-(4-Butyldecanoyl)-N-methylglycyl)oxy)-6-((4-((tert-butyldiphenylsilyl)oxy)-butyl) (methyl)amino)undecyl 4-pentylundecanoate. ¹H NMR (400 MHz, CDCl₃, rotamers) δ 7.72-7.60 (m, 4H), 7.46-7.32 (m, 6H), 4.21-3.99 (m, 6H), 3.67 (m, 2H), 3.07 & 2.96 (s, 3H), 2.42-2.10 (m, 10H), 1.86 (m, 1H), 1.76-1.47 (m, 15H), 1.38-1.13 (m, 46H), 1.04 (s, 9H), 0.90-0.85 (tr, 12H).

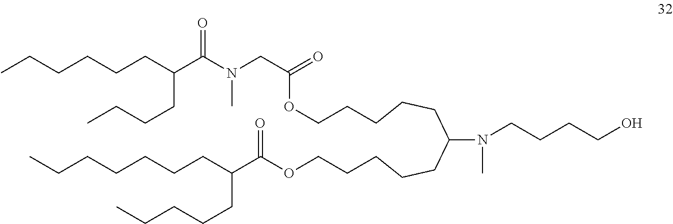 [SMILES: CCCCCCCC(CCCCC)C(=O)OCCCCCC(CCCCCOC(=O)CN(C)C(=O)C(CCCC)CCCCCC)N(C)CCCCO]

(xxxi) 11-((N-(4-Butyldecanoyl)-N-methylglycyl)oxy)-6-((4-hydroxybutyl)(methyl)amino)-undecyl 4-pentylundecanoate (32). ¹H NMR (400 MHz, CDCl₃, rotamers) δ4.21-4.02 (m, 6H), 3.72 (m, 2H), 3.23-2.94 (m, 6H), 2.78 (s, 3H), 2.39-2.16 (m, 4H), 1.94 (m, 2H), 1.81-1.63 (m, 10H), 1.61-1.52 (m, 6H), 1.50-1.37 (m, 9H), 1.34-1.17 (m, 34H), 0.91-0.84 (tr, 12H).

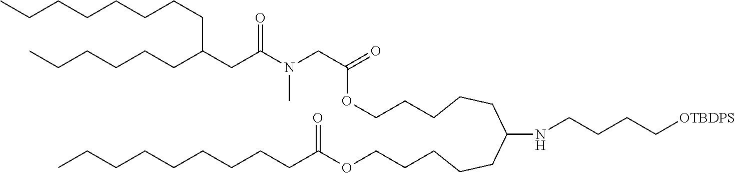

(xxxii) 6-((4-((tert-Butyldiphenylsilyl)oxy)butyl)amino)-11-((N-(3-hexylundecanoyl)-N-methylglycyl)oxy)undecyl decanoate. From ketone 128 and amine 134. ¹H NMR (400 MHz, CDCl₃, rotamers) δ7.70-7.60 (m, 4H), 7.46-7.33 (m, 6H), 4.19-4.00 (m, 6H), 3.66 (tr, J=5.76, 2H), 3.07 & 2.97 (s, 3H), 2.67-2.07 (m, 8H), 1.92 (m, 1H), 1.71-1.52 (m, 12H), 1.43-1.19 (m, 46H), 1.04 (s, 9H), 0.89-0.85 (br t, 9H).

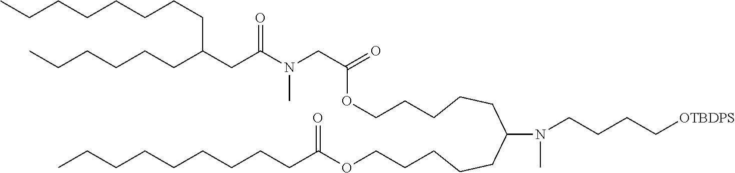

(xxxiii) 6-((4-((tert-Butyldiphenylsilyl)oxy)butyl)(methyl)amino)-11-((N-(3-hexylundecan-oyl)-N-methylglycyl)oxy)undecyl decanoate. ¹H NMR (400 MHz, CDCl₃, rotamers) δ7.70-7.62 (m, 4H), 7.44-7.32 (m, 6H), 4.21-3.97 (m, 6H), 3.67 (t, 2H), 3.07 & 2.97 (s, 3H), 2.52-1.91 (m, 11H), 1.77-1.46 (m, 12H), 1.38-1.19 (m, 46H), 1.04 (s, 9H), 0.89-0.85 (br t, 9H).

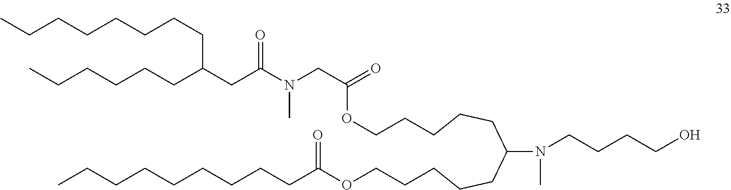 [SMILES: CCCCCCCCCC(=O)OCCCCCC(CCCCCOC(=O)CN(C)C(=O)CC(CCCCCC)CCCCCCCC)N(C)CCCCO]

(xxxiv) 11-((N-(3-Hexylundecanoyl)-N-methylglycyl)oxy)-6-((4-hydroxybutyl)(methyl)-amino)-undecyl decanoate (33). ¹H NMR (400 MHz, CDCl₃, rotamers) δ 4.21-3.99 (m, 6H), 3.72 (m, 2H), 3.24-2.94 (m, 6H), 2.76 (s, 3H), 2.34-2.11 (m, 4H), 1.99-1.85 (m, 3H), 1.79-1.54 (m, 13H), 1.49-1.38 (m, 8H), 1.32-1.20 (m, 36H), 0.9-0.84 (br t, 9H).

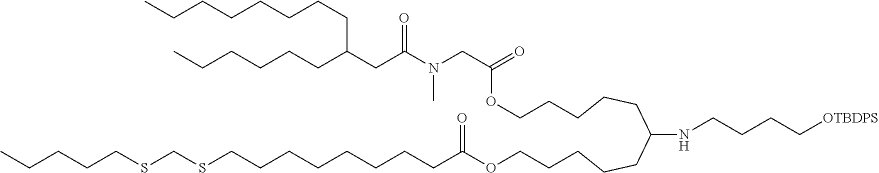

(xxxv) 6-((4-((tert-Butyldiphenylsilyl)oxy)butyl)amino)-11-((N-(3-hexylundecanoyl)-N-methylglycyl)oxy)undecyl 9-(((pentylthio)methyl)thio)nonanoate. From ketone 140 and amine 134. ¹H NMR (400 MHz, CDCl₃, rotamers) δ7.70-7.61 (m, 4H), 7.47-7.32 (m, 6H), 4.21-3.97 (m, 6H), 3.65 (s, 2H), 3.07 & 2.97 (s, 3H), 2.66-2.55 (m, 7H), 2.40 (tr, J=7.3, 2H), 2.31-2.11 (m, 4H), 1.92 (m, 1H), 1.66-1.55 (m, 20H), 1.42-1.22 (m, 43H), 1.04 (s, 9H), 0.9-0.86 (br t, 9H).

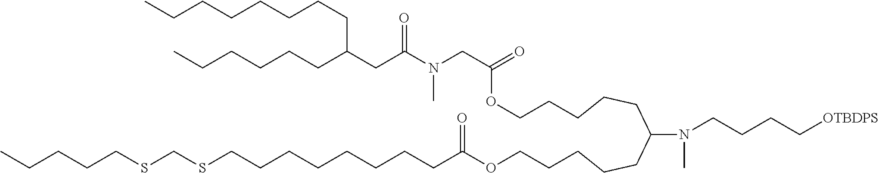

(xxxvi) 6-((4-((tert-Butyldiphenylsilyl)oxy)butyl)(methyl)amino)-11-((N-(3-hexylundecan-oyl)-N-methylglycyl)oxy)undecyl 9-(((pentylthio)methyl)thio)nonanoate. ¹H NMR (400 MHz, CDCl₃, rotamers) δ7.70-7.61 (m, 4H), 7.45-7.33 (m, 6H), 4.19-4.00 (m, 6H), 3.65 (m, 2H), 3.07 & 2.97 (s, 3H), 4H), 2.42 (m, 3H), 2.32-2.01 (m, 10H), 1.92 (m, 1H), 1.68-1.49 (m, 16H), 1.46-1.20 (m, 45H), 1.04 (s, 9H), 0.93-0.84 (m, 9H).

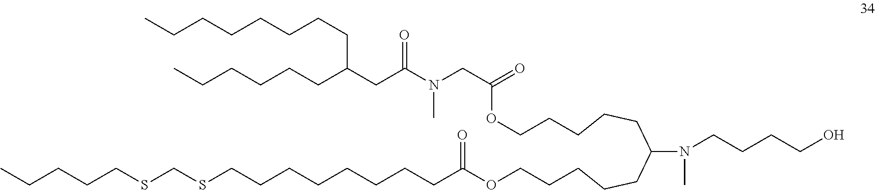 [SMILES: CCCCCCCCC(CCCCCC)CC(=O)N(C)CC(=O)OCCCCCC(CCCCCOC(=O)CCCCCCCCSCSCCCCC)N(C)CCCCO]

(xxxvii) 11-((N-(3-Hexylundecanoyl)-N-methylglycyl)oxy)-6-((4-hydroxybutyl)(methyl)-amino)-undecyl 9-(((pentylthio)methyl)thio)nonanoate (34). ¹H NMR (400 MHz, CDCl₃, rotamers) δ4.21-4.00 (m, 6H), 3.65 (s, 2H), 3.25-2.95 (m, 6H), 2.77 (s, 3H), 2.62 (tr, J=7.2, 4H), 2.32-2.11 (m, 4H), 2.00-1.82 (m, 3H), 1.81-1.50 (m, 18H), 1.48-1.21 (m, 44H), 0.95-0.80 (br t, 9H).

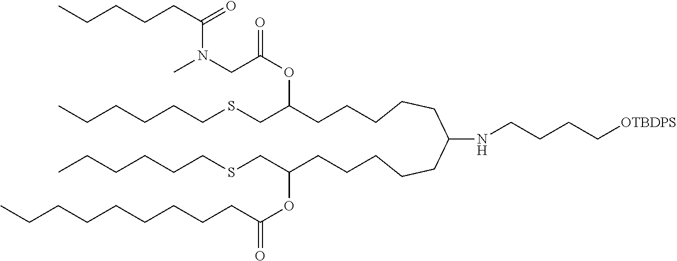

(xxxviii) 10-(6-((N-Hexanoyl-N-methylglycyl)oxy)-7-(hexylthio)heptyl)-2,2-dimethyl-3,3-diphenyl-4-oxa-18-thia-9-aza-3-silatetracosan-16-yl decanoate. From ketone 127 and amine 134. ¹H NMR (400 MHz, CDCl₃, rotamers) δ 7.66 (m, 4H), 7.47-7.33 (m, 6H), 5.10-4.88 (m, 2H), 4.26-4.01 (m, 2H), 3.66 (t, J=5.8, 2H), 3.06 & 2.98 (s, 3H), 2.68-2.60 (m, 4H), 2.58-2.50 (m, 6H), 2.43 (br, 1H), 2.36 (tr, J=7.7, 2H), 2.33-2.22 (m, 3H), 1.77-1.48 (m, 16H), 1.41-1.21 (m, 44H), 1.04 (s, 9H), 0.91-0.85 (br t, 12H).

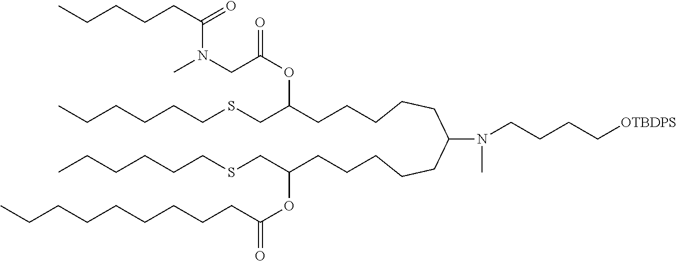

(xxxix) 10-(6-((N-Hexanoyl-N-methylglycyl)oxy)-7-(hexylthio)heptyl)-2,2,9-trimethyl-3,3-diphenyl-4-oxa-18-thia-9-aza-3-silatetracosan-16-yl decanoate. ¹H NMR (400 MHz, CDCl₃, rotamers) δ 7.66 (m, 4H), 7.44-7.34 (m, 6H), 5.11-4.87 (m, 2H), 4.25-4.00 (m, 2H), 3.66 (t, J=5.8, 2H), 3.06 & 2.98 (s, 3H), 2.68-2.59 (m, 4H), 2.56-2.49 (m, 4H), 2.41-2.21 (m, 7H), 2.15 (s, 3H), 1.71-1.46 (m, 17H), 1.40-1.20 (m, 43H), 1.04 (s, 9H), 0.92-0.83 (br t, 12H).

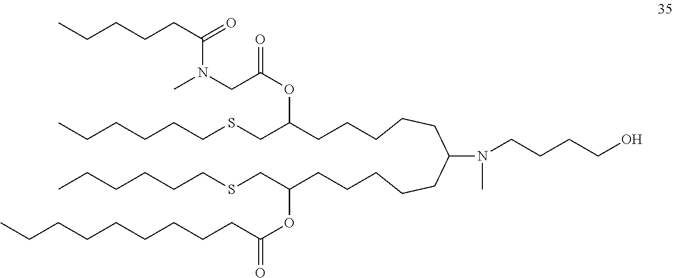 [SMILES: CCCCCCCCCC(=O)OC(CCCCCC(CCCCCC(CSCCCCCC)OC(=O)CN(C)C(=O)CCCCC)N(C)CCCCO)CSCCCCCC]

(x) 14-((N-Hexanoyl-N-methylglycyl)oxy)-1,15-bis(hexylthio)-8-((4-hydroxybutyl)-(methyl)amino)pentadecan-2-yl decanoate (35). ¹H NMR (400 MHz, CDCl₃, rotamers) δ 5.02 (m, 1H), 4.94 (m, 1H), 4 0.32-3.87 (m, 2H), 3.77 (m, 1H), 3.67 (m, 1H), 3.25-2.94 (6H), 2.84-2.72 (m, 3H), 2.64 (d, J=6 Hz, 4H), 2.58-2.50 (m, 4H), 2.37 (m, 2H), 2.31 (m, 2H), 2.00 (1H), 1.87 (2H), 1.79-1.50 (m, 16H), 1.47-1.21 (m, 40H), 0.94-0.81 (br t, 12H).

(f) Preparation of Lipids of the Type 36-38 from Appropriate Ketones.

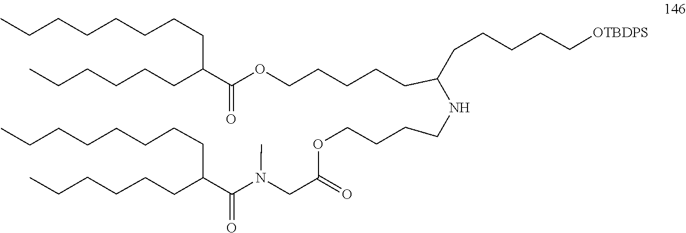

(i) 11-((tert-Butyldiphenylsilyl)oxy)-6-((4-((N-(2-hexyldecanoyl)-N-methylglycyl)oxy)-butyl)amino)undecyl 2-hexyldecanoate (146). To a solution of ketone 118 (750 mg, 1.2 mmol, 1 equiv), amine 143 (939 mg, 2.4 mmol, 2 equiv), and acetic acid (1 drop) in 1,2-dichloroethane (20 mL) was added sodium triacetoxyborohydride (381 mg, 1.8 mmol, 1.5 equiv). The mixture was stirred at rt for 16 h, whereupon the reaction was complete (TLC). The solution was diluted with CH₂Cl₂ (50 mL) and washed with aq. saturated sodium bicarbonate solution (50 mL). The solution was dried (Na₂SO₄) and concentrated. The colorless residual oil was purified by chromatography on silica gel (40-50% EtOAc in hexane) to give 146 as a colorless gummy oil (quant.) ¹H NMR (400 MHz, CDCl₃, rotamers) δ7.75-7.62 (m, 4H), 7.48-7.33 (m, 6H), 4.23-4.01 (m, 6H), 3.67 (t, J=6.4 Hz, 2H), 3.08 (s, 3H), 2.69 (td, J=8.5, 4.3 Hz, 1H), 2.59 (td, J=7.1, 3.9 Hz, 2H), 2.46 (s, 1H), 2.33 (tt, J=9.0, 5.3 Hz, 1H), 1.76-1.17 (m, 66H), 1.07 (s, 9H), 0.92-0.86 (br t, 12H).

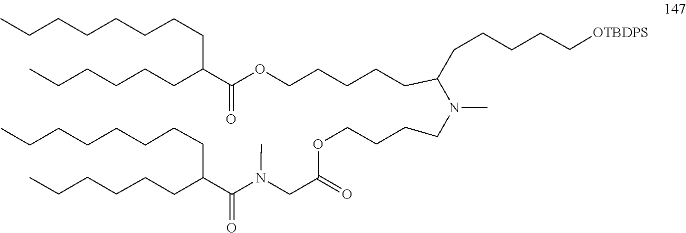

(ii) 11-((tert-Butyldiphenylsilyl)oxy)-6-((4-((N-(2-hexyldecanoyl)-N-methylglycyl)oxy)butyl)-(methyl)amino)undecyl 2-hexyldecanoate (147). A mixture of 146 (850 mg, 0.8 mmol), aq. formaldehyde (387%, 4 mL) and NaBH(OAc)₃ (681 mg, 3.2 mmol) in THF (8 mL) was stirred under inert atmosphere at room temperature for 3 days. The reaction was then quenched with sat. aq. NaHCO₃ (3 mL), diluted with water (5 mL) and extracted with DCM (3×10 mL). The combined extracts were dried (Na₂SO₄) and concentrated. The residue was purified by silica chromatography (0-5% MeOH in DCM) to yield the tertiary amine 147 (740 mg, 86%) as an oil. ¹H NMR (400 MHz, CDCl₃, rotamers) δ7.76-7.63 (m, 4H), 7.49-7.33 (m, 6H), 4.23-3.99 (m, 6H), 3.67 (t, J=6.5 Hz, 2H), 3.07 (d, J=40.5 Hz, 3H), 2.70 (dq, J=8.5, 4.4 Hz, 1H), 2.45-2.21 (m, 4H), 2.15 (d, J=3.7 Hz, 3H), 1.79-1.12 (m, 68H), 1.07 (s, 9H), 0.92-0.85 (br t, 12H).

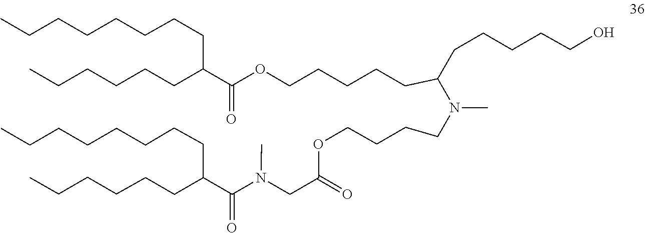 [SMILES: CCCCCCCCC(CCCCCC)C(=O)OCCCCCC(CCCCCO)N(C)CCCCOC(=O)CN(C)C(=O)C(CCCCCC)CCCCCCCC]

(iii) 6-((4-((N-(2-Hexyldecanoyl)-N-methylglycyl)oxy)butyl)(methyl)amino)-11-hydroxy-undecyl 2-hexyldecanoate (36) To a solution of 147 (251 mg, 0.24 mmol) in CH₂Cl₂ (4 mL) was added HF-Pyridine (0.3 mL) at 0° C. under nitrogen atmosphere. The reaction was warmed to room temperature and stirred for 18 hours. Water (7 mL) was added, and the mixture was extracted with CH₂Cl₂ (3×7 mL). The combined extracts were dried (Na₂SO₄) and concentrated. The residue was purified by silica chromatography (0-5% MeOH in DCM) to yield lipid 36 (156 mg, 80%) as an oil. ¹H NMR (400 MHz, CDCl₃, rotamers) δ4.28-3.97 (m, 6H), 3.68 (m, 2H), 3.17 (s, 6H), 2.86-2.57 (m, 4H), 2.33 (m, 2H), 1.92-1.18 (m, 66H), 0.96-0.81 (m, 12H).

The following compounds were prepared by the same method:

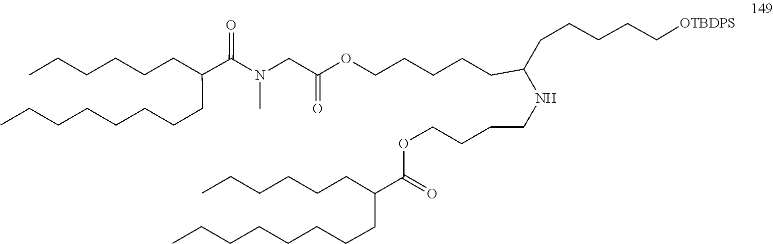

(iv) 4-((21-Hexyl-2,2,19-trimethyl-17,20-dioxo-3,3-diphenyl-4,16-dioxa-19-aza-3-silanona-cosan-10-yl)amino)butyl 2-hexyldecanoate (149). From ketone 148 and amine 145. ¹H NMR (400 MHz, CDCl₃, rotamers) δ7.80-7.63 (m, 4H), 7.48-7.34 (m, 6H), 4.21-4.01 (m, 6H), 3.67 (t, J=6.4 Hz, 2H), 3.08 (m, 3H), 2.70 (tt, J=8.5, 5.2 Hz, 1H), 2.60 (t, J=7.1 Hz, 2H), 2.46 (s, 1H), 2.33 (tt, J=8.8, 5.3 Hz, 1H), 1.82-1.17 (m, 67H), 1.07 (s, 9H), 0.94-0.85 (m, 12H).

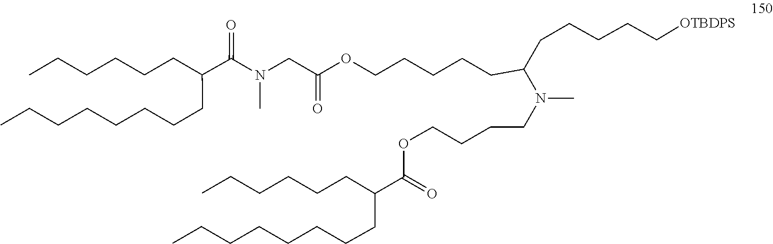

(v) 4-((21-hexyl-2,2,19-trimethyl-17,20-dioxo-3,3-diphenyl-4,16-dioxa-19-aza-3-silanonacos-an-10-yl)(methyl)amino)butyl 2-hexyldecanoate (150). ¹H NMR (400 MHz, CDCl₃, rotamers) δ 7.75-7.63 (m, 4H), 7.52-7.34 (m, 6H), 4.26-3.96 (m, 6H), 3.67 (t, J=6.5 Hz, 2H), 3.08 (d, J=40.5 Hz, 3H), 2.70 (ddd, J=8.5, 5.2, 3.1 Hz, 1H), 2.46-2.25 (m, 4H), 2.15 (s, 3H), 1.80-1.09 (m, 66H), 1.07 (s, 9H), 0.90 (br t, 12H).

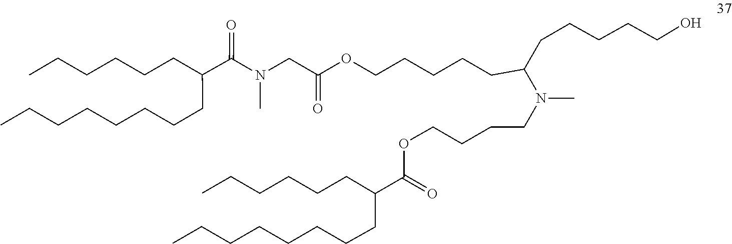 [SMILES: CCCCCCCCC(CCCCCC)C(=O)OCCCCN(C)C(CCCCCO)CCCCCOC(=O)CN(C)C(=O)C(CCCCCC)CCCCCCCC]

(vi) 4-((1-((N-(2-Hexyldecanoyl)-N-methylglycyl)oxy)-11-hydroxyundecan-6-yl)(methyl)-amino)butyl 2-hexyldecanoate (37). ¹H NMR (400 MHz, CDCl₃, rotamers) δ 4.23-4.05 (m, 6H), 3.68 (t, J=6.1 Hz, 2H), 3.09 (m, 6H), 2.87-2.65 (m, 4H), 2.33 (tt, J=8.7, 5.4 Hz, 1H), 1.94-1.13 (m, 68H), 0.94-0.84 (m, 12H).

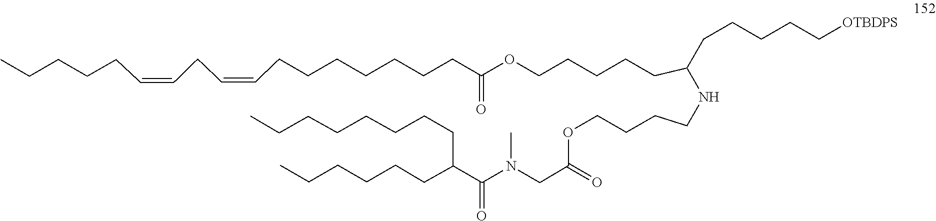

(vii) 11-((tert-Butyldiphenylsilyl)oxy)-6-((4-((N-(2-hexyldecanoyl)-N-methylglycyl)oxy)-butyl)amino)undecyl (9Z,12Z)-octadeca-9,12-dienoate (152). From ketone 151 and amine 143. ¹H NMR (400 MHz, CDCl₃, rotamers) δ7.75-7.64 (m, 4H), 7.48-7.35 (m, 6H), 5.47-5.28 (m, 4H), 4.22-4.00 (m, 6H), 3.67 (t, J=6.4 Hz, 2H), 3.08 (d, J=41.9 Hz, 3H), 2.79 (t, J=6.4 Hz, 2H), 2.75-2.53 (m, 3H), 2.45 (d, J=6.0 Hz, 1H), 2.31 (t, J=7.6 Hz, 2H), 2.13-1.99 (m, 4H), 1.82-1.13 (m, 58H), 1.07 (s, 9H), 0.92-0.86 (br t, 9H).

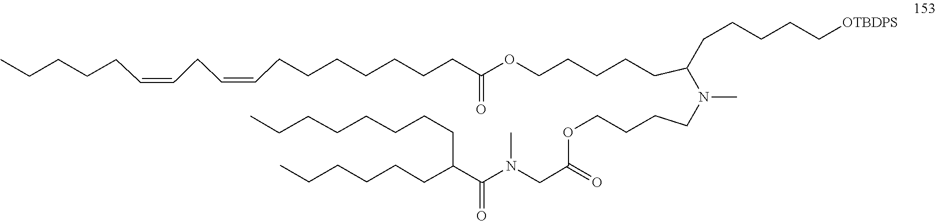

(viii) 11-((tert-Butyldiphenylsilyl)oxy)-6-((4-((N-(2-hexyldecanoyl)-N-methylglycyl)oxy)-butyl)(methyl)amino)undecyl (9Z,12Z)-octadeca-9,12-dienoate (153). ¹H NMR (400 MHz, CDCl₃, rotamers) δ7.74-7.64 (m, 4H), 7.48-7.34 (m, 6H), 5.47-5.24 (m, 4H), 4.21-3.99 (m, 6H), 3.67 (t, J=6.5 Hz, 2H), 3.07 (d, J=40.8 Hz, 3H), 2.79 (t, J=6.4 Hz, 2H), 2.70 (m, 1H), 2.44-2.24 (m, 5H), 2.15 (d, J=3.6 Hz, 3H), 2.07 (d, J=6.9 Hz, 5H), 1.74-1.12 (m, 57H), 1.07 (s, 9H), 0.92-0.86 (br t, 9H).

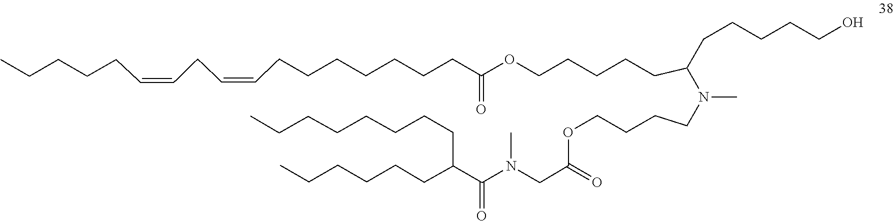 [SMILES: CCCCC/C=C\C/C=C\CCCCCCCC(=O)OCCCCCC(CCCCCO)N(C)CCCCOC(=O)CN(C)C(=O)C(CCCCCC)CCCCCCCC]

(ix) 6-((4-((N-(2-Hexyldecanoyl)-N-methylglycyl)oxy)butyl)(methyl)amino)-11-hydroxy-undecyl (9Z,12Z)-octadeca-9,12-dienoate (38). ¹H NNR (400 MHz, CDCl₃, rotamers) δ 5.37 (m 4H) 4.29-4.00 (m, 6H), 3.68 (m, 2H), 3.17 (s, 6H), 2.86-2.63 (m, 6H), 2.31 (t, J=7.6 Hz, 2H), 2.07 (q, J=6.9 Hz, 4H), 1.91-1.16 (m, 58H), 0.90 (br t, 9H).

(g) Procedure for the Double N-Alkylation of a Primary Amine with an Alkyl Bromide Leading to a Tertiary Amine.

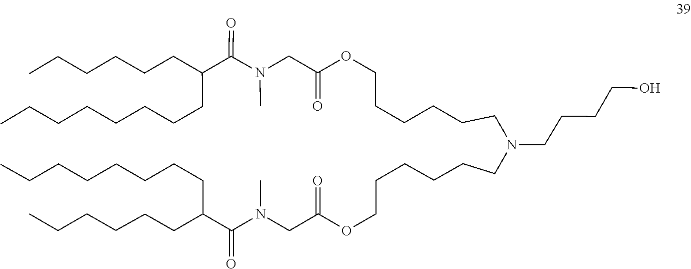 [SMILES: CCCCCCCCC(CCCCCC)C(=O)N(C)CC(=O)OCCCCCCN(CCCCO)CCCCCCOC(=O)CN(C)C(=O)C(CCCCCC)CCCCCCCC]

(i) ((4-hydroxybutyl)azanediyl)bis(hexane-6,1-diyl) bis(2-(2-hexyl-N-methyldecanamido)-acetate) (39). A mixture of bromide 154 (172 mg, 0.351 mmol), 4-amino-1-butanol (16 mg, 0.175 mmol) and K₂CO₃ (51 mg, 0.368 mmol) in MeCN (3.5 mL) was stirred at 80° C. in a sealed reaction vessel for 18 hours. The mixture was cooled, diluted with water (5 mL) and extracted with CH₂Cl₂ (3×5 mL). The combined extracts were dried (Na₂SO₄) and concentrated. The residue was purified by silica chromatography (0-5% MeOH in DCM) to yield lipid 39 (98 mg, 62%) as an oil. ¹H NMR (400 MHz, CDCl₃, rotamers) δ4.13-4.10 (m, 8H), 3.61 (br m, 2H), 3.12 & 3.01 (s, 6H, rotamer, 3:1), 2.72-2.65 (br m, 7H), 1.74 (br s, 1H), 1.68-1.61 (m, 13H), 1.45-1.26 (m, 56H), 0.89-0.86 (br t, 12H, J=7.15 Hz).

The following compound was prepared by the same method:

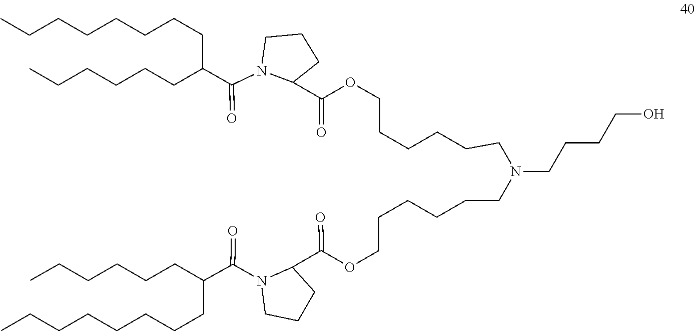 [SMILES: CCCCCCCCC(CCCCCC)C(=O)N1CCCC1C(=O)OCCCCCCN(CCCCO)CCCCCCOC(=O)C1CCCN1C(=O)C(CCCCCC)CCCCCCCC]

(ii) ((4-Hydroxybutyl)azanediyl)bis(hexane-6,1-diyl) bis((2-hexyldecanoyl)prolinate) (40). From bromide 155 and 4-amino-1-butanol. ¹H NMR (400 MHz, CDCl₃, rotamers) δ4.50-4.50 (m, 2H), 4.20-4.03 (m, 4H), 3.74-3.51 (m, 6H), 2.94 (s, 5H), 2.48 (m, 2H), 2.30-1.53 (m, 25H), 1.48-1.13 (m, 52H), 0.89 (br t, 12H).

(h) Procedure for the Sequential N-Alkylation of a Primary Amine with Two Different Alkyl Halides Leading to a Tertiary Amine.

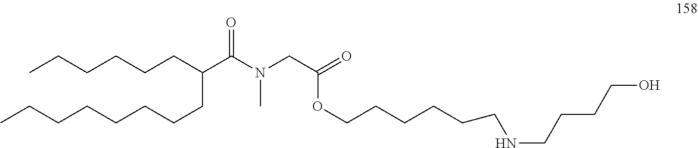 [SMILES: CCCCCCCCC(CCCCCC)C(=O)N(C)CC(=O)OCCCCCCNCCCCO]

(i) 6-((4-Hydroxybutyl)amino)hexyl N-(2-hexyldecanoyl)-N-methylglycinate (158). A solution of bromide 154 (229 mg, 0.468 mmol), 4-amino-1-butanol (44 mg, 0.491 mmol) and K₂CO₃ (68 mg, 0.491 mmol) in DMF (5 mL) was stirred at room temperature for 18 hours under nitrogen atmosphere. The mixture was then diluted with water (10 mL) and extracted with Et₂O (3×10 mL). The combined extracts were washed with brine (10 mL), dried (Na₂SO₄) and concentrated. The residue was purified by silica chromatography (0-45% MeOH in CH₂Cl₂) to yield secondary amine 158 (136 mg, 58%). ¹H NMR (400 MHz, CDCl₃, rotamers) δ4.18-4.05 (m, 4H), 3.60-3.53 (m, 2H), 3.12 & 3.01 (s, 3H, rotamers 3:1), 2.74-2.55 (m, 4H), 1.77-1.11 (m, 37H), 0.88 (br t, 6H).

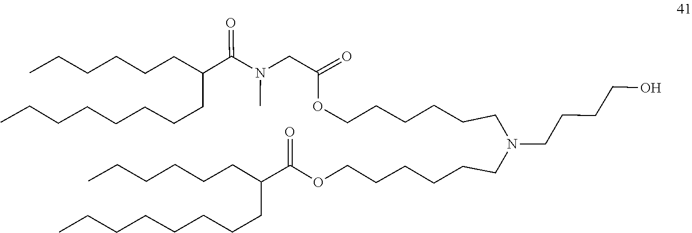 [SMILES: CCCCCCCCC(CCCCCC)C(=O)OCCCCCCN(CCCCO)CCCCCCOC(=O)CN(C)C(=O)C(CCCCCC)CCCCCCCC]

(ii) 6-((6-((N-(2-Hexyldecanoyl)-N-methylglycyl)oxy)hexyl)(4-hydroxybutyl)amino)hexyl 2-hexyldecanoate (41). A mixture of amine 158 (126 mg, 0.252 mmol), bromide 156 (116 mg, 0.278 mmol) and K₂CO₃ (42 mg, 0.302 mmol) in MeCN (2.5 mL) was stirred at 80° C. in a sealed reaction vessel for 18 hours. The mixture was cooled, diluted with water (5 mL) and extracted with CH₂Cl₂ (3×5 mL). The combined extracts were dried (Na₂SO₄) and concentrated. The residue was purified by silica chromatography (0-5% MeOH in CH₂Cl₂) to yield lipid 33 (135 mg, 64%) as an oil. ¹H NMR (400 MHz, CDCl₃, rotamers) δ4.14-4.05 (m, 6H), 3.59 (br m, 2H), 3.13 & 3.02 (s, 3H, rotamers, 3:1), 2.73-2.28 (br m, 8H), 1.69-1.57 (m, 16H), 1.46-1.26 (m, 52H), 0.90-0.87 (m, 12H).

The following compounds were prepared by the same method:

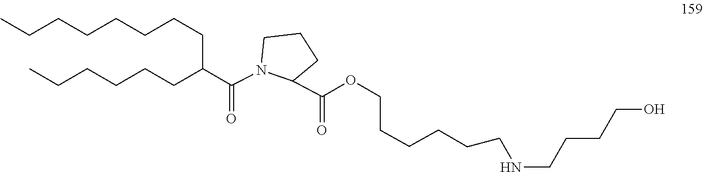 [SMILES: CCCCCCCCC(CCCCCC)C(=O)N1CCCC1C(=O)OCCCCCCNCCCCO]

(iii) 6-((4-Hydroxybutyl)amino)hexyl (2-hexyldecanoyl)prolinate (159). From 4-amino-1-butanol and bromide 155. ¹H NMR (400 MHz, CDCl₃, rotamers) δ 4.50 (m, 1H), 4.10 (m, 2H), 3.75-3.49 (m, 4H), 2.70-2.56 (m, 4H), 2.48 (m, 1H), 2.28-1.87 (m, 4H), 1.74-1.17 (m, 36H), 0.89 (br t, 6H).

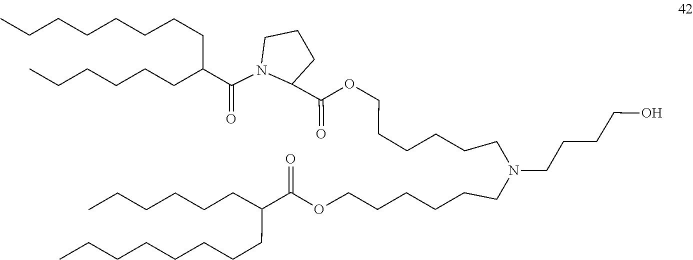 [SMILES: CCCCCCCCC(CCCCCC)C(=O)OCCCCCCN(CCCCO)CCCCCCOC(=O)C1CCCN1C(=O)C(CCCCCC)CCCCCCCC]

(iv) 6-((6-((2-Hexyldecanoyl)oxy)hexyl)(4-hydroxybutyl)amino)hexyl (2-hexyldecanoyl)-prolinate (42). From amine 159 and bromide 156. ¹H NMR (400 MHz, CDCl₃, rotamers) δ 4.50 (m, 1H), 4.21-4.00 (m, 4H), 3.75-3.49 (m, 4H), 2.49 (m, 6H), 2.37-1.80 (m, 6H), 1.78-1.17 (m, 68H), 0.89 (br t, 12H).

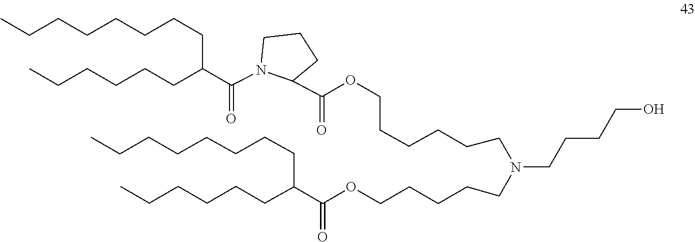 [SMILES: CCCCCCCCC(CCCCCC)C(=O)OCCCCCN(CCCCO)CCCCCCOC(=O)C1CCCN1C(=O)C(CCCCCC)CCCCCCCC]

(v) 6-((5-((2-Hexyldecanoyl)oxy)pentyl)(4-hydroxybutyl)amino)hexyl (2-hexyldecanoyl)-prolinate (43). From amine 159 and chloride 157. ¹H NMR (400 MHz, CDCl₃) δ 4.49 (dd, J=8.6, 3.9 Hz, 1H), 4.09 (t, J=6.5 Hz, 4H), 3.64 (m, 4H), 3.07 (m, 6H), 2.49 (m, 1H), 2.37-1.51 (m, 24H), 1.51-1.06 (m, 47H), 0.89 (br t, 12H).

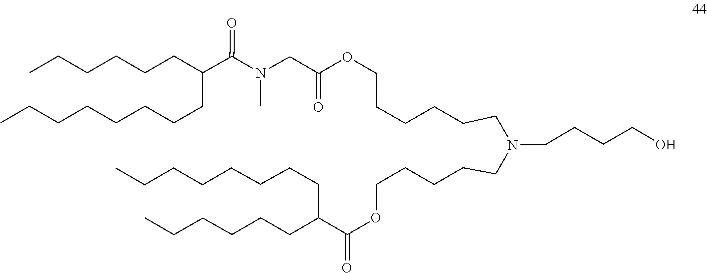 [SMILES: CCCCCCCCC(CCCCCC)C(=O)OCCCCCN(CCCCO)CCCCCCOC(=O)CN(C)C(=O)C(CCCCCC)CCCCCCCC]

(vi) 5-((6-((N-(2-Hexyldecanoyl)-N-methylglycyl)oxy)hexyl)(4-hydroxybutyl)amino)pentyl 2-hexyldecanoate (44). From amine 158 and chloride 157. ¹H NMR (400 MHz, CDCl₃, rotamers) δ 4.12 (m, 6H), 3.75 (t, J=5.6 Hz, 2H), 3.09 (m, 9H), 2.71 (s, 1H), 2.33 (s, 2H), 1.28 (m, 67H), 0.90 (br t, 12H).

### Example 2: Results for In Vivo Delivery of mRNA to the Liver and Spleen Relative to the MC3, ALC-0315 and SM-102 Benchmark for LNPs Comprising Ionizable Lipids of the Disclosure

LNP formulations containing 50/10/38.5/1.5 mol % of lipids 1, 3-7, 16-19, 21-27 and 31-36 ionizable lipid/DSPC/chol/PEG-DMG and mRNA encoding luciferase were tested for in vivo biodistribution in the liver and spleen after injection to CD-1 mice. The mRNA dose was 1 mg/kg. Luminescence intensity in the liver and spleen was measured at 4 hours post-injection.

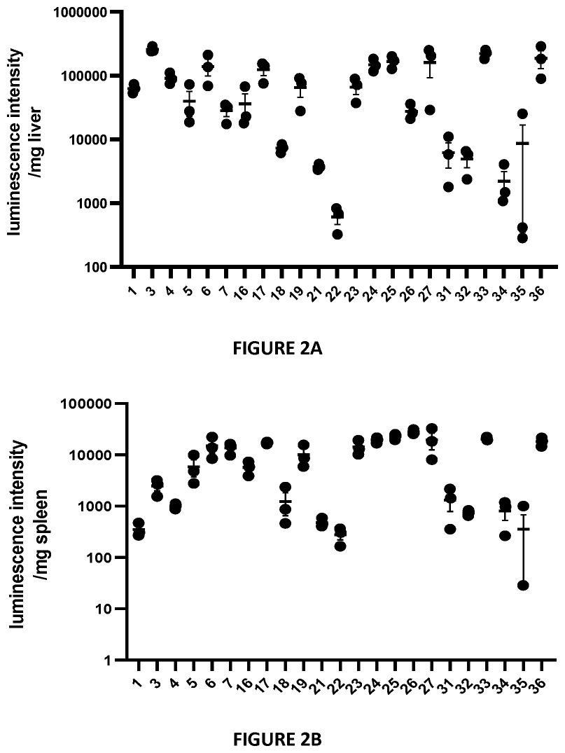

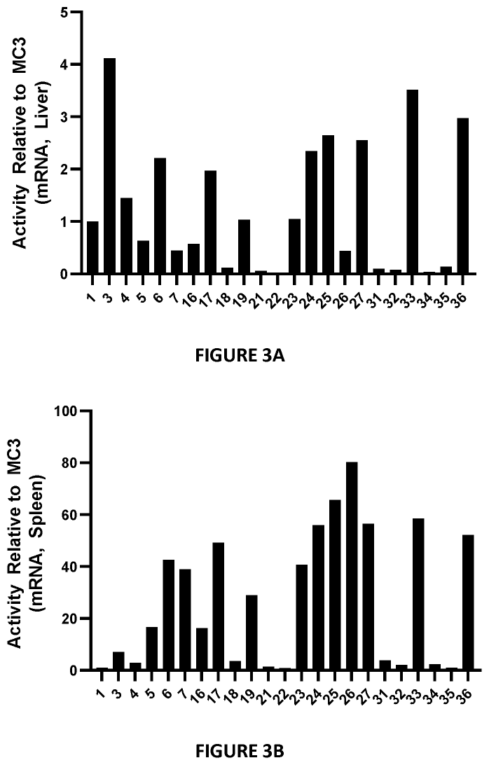

The results in FIG. 2A and FIG. 3A show that luminescence intensity per mg liver was higher for the amino acid lipids 6, 17, 19, 23, 24, 25, 27, 33 and 36 than the MC3 benchmark (lipid 1). Results for luminescence intensity per mg spleen were found to be particularly efficacious (FIG. 2B and FIG. 3B) relative to benchmark lipids 1, 3 and 4.

1. A lipid having the structure of Formula A: 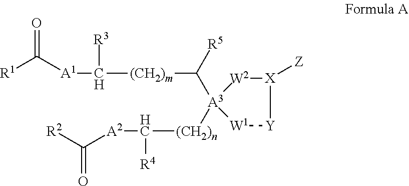
or a pharmaceutically acceptable salt thereof,
wherein indices m and n vary, independently, from 1 to 8;
groups A¹ and A² are both present or both absent, and
if groups A¹ and A² are both present, they are both O, and
at least one of R¹ and R² is an amino acid-derived moiety having the structure Formula B 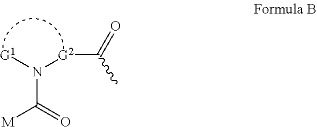
wherein the wavy bond connects to A¹ and/or A² (that is, O) in Formula A,
G¹ is a C₁-C₆ alkyl or cycloalkyl, optionally comprising one or more heteroatoms, optionally selected from N, O and/or S;
G² is (CR^{a}R^{b})ₚ, wherein R^{a} and R^{b} are, independently, H or C₁-C₆ alkyl or cycloalkyl, and index p can range from p=1 to p=5;
the dotted semicircle between G¹ and G² signifies that one of the atoms that are part of G¹ may be bonded to one of the atoms that are part of G², so as to create a ring structure that encompasses the N atom;
M is optionally
a linear or branched alkyl group having from 10 to 20 carbon atoms; optionally comprising from 1 to 3 C═C double bonds of E or Z geometry; optionally comprising 1 to 3 heteroatoms such as N, O, S, which are optionally part of a functional group that is an ester, ether, amide, amine or thioether; and optionally comprising additional OH, O-alkyl, S-alkyl substituents, or
a moiety of Formula C, 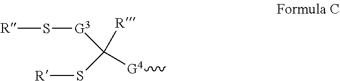
wherein R′ and R″ are, independently, a linear or branched alkyl group comprising from 3 to 12 C atoms, optionally incorporating from 1 to 3 C═C double bonds of E or Z geometry; optionally incorporating 1 to 3 heteroatoms such as N, O, S, which are optionally part of a functional group that is an ester, ether, amide, amine or thioether;
and optionally incorporating additional OH, O-alkyl, S-alkyl substituents;
R′″ is H or a linear, branched, or cyclic alkyl group comprising from 1 to 6 C atoms, and optionally incorporating heteroatoms such as N, O, S;
G³ and G⁴ are, independently, a group of structure (CR^{a}R^{b})_{q} with R^{a} and R^{b} independently equal to H or small C₁-C₅ alkyl or cycloalkyl, and with index q varying from q=0 (in which case G³/G⁴ is/are absent) to q=6, and
if only one of R¹ and R² is a group as described above, then the other one of R¹ and R² is a linear or branched or cyclic alkyl group comprising from 4 to 30 C atoms, optionally comprising heteroatoms such as N, O, S, optionally comprising from 1 to 3 C═C double bonds of E or Z geometry, optionally comprising substituents such as alkyl or cycloalkyl, O-alkyl, S-alkyl, wherein the alkyl or cycloalkyl or O-alkyl or S-alkyl group comprises from 1 to 8 carbon atoms;
R³ and R⁴ are, independently, H or a group of structure R⁶—S—CH₂, wherein R⁶ is a C₁-C₁₂ alkyl or cycloalkyl group;
R⁵ is H or a C₁-C₆ alkyl group optionally comprising a C═C double bond of E or Z geometry, optionally comprising up to 3 heteroatoms selected from N, O, S,
if groups A¹ and A² are both absent, then
R³ and R⁴ are both H;
R⁵ is H or a C₁-C₆ alkyl group optionally comprising a C═C double bond of E or Z geometry, optionally comprising up to 3 heteroatoms selected from N, O, S;
at least one of R¹ and R² is an amino acid-derived moiety as depicted in Formula D, 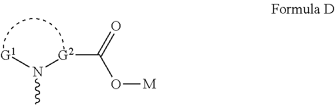
wherein the wavy bond connects the N atom to the C═O group in Formula A,
G¹ is a C₁-C₆ small alkyl or cycloalkyl, optionally comprising one or more heteroatoms such as N, O, S;
G² is (CR^{a}R^{b})ₚ, wherein R^{a} and R^{b} are, independently, H or small C₁-C₆ alkyl or cycloalkyl, and index p can range from p=1 to p=5;
the dotted semicircle between G¹ and G² denotes that an atom of G¹ is optionally bonded to an atom of G², so as to form a ring structure that encompasses the N atom;
M is a linear or branched alkyl group having from 10 to 20 carbon atoms; optionally comprising from 1 to 3 C═C double bonds of E or Z geometry; optionally comprising 1 to 3 heteroatoms selected from N, O and/or S, which are optionally part of a functional group that is an ester, ether, amide, amine or thioether; and optionally incorporating additional OH, O-alkyl, S-alkyl substituents, or
a moiety of Formula C, 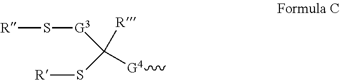
wherein R′, R″, R′″, G³ and G⁴ are as defined above, and
if only one of R¹ and R² is a group as described above, then the other one of R¹ and R² is a group of the type R^{c}—O, wherein the O atom is bound to the C═O group in Formula A, and R^{c} is a linear or branched or cyclic alkyl group comprising from 4 to 30 C atoms, optionally comprising heteroatoms selected from N, O and/or S, optionally comprising from 1 to 3 C═C double bonds of E or Z geometry, optionally comprising substituents such as alkyl or cycloalkyl, O-alkyl, S-alkyl, wherein the alkyl or cycloalkyl or O-alkyl or S-alkyl group comprises from 1 to 8 carbon atoms,
A² is either C or N, and if A² is C, then
R⁵ is H or a C₁-C₅ alkyl;
W¹ and Y are either bonded to each other or not bonded to each other (as indicated by the dashed bond), and:
if W¹ and Y are bonded to each other then:
W¹ is O or S;
W² is O or S;
X is CH;
Y is (CH₂)ₚ, wherein p is 1 or 2;
Z is a group selected from structures a-c below, wherein the wavy line represents the bond to X 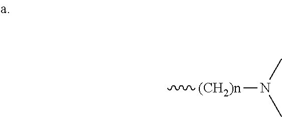
type 2 ionizable head; 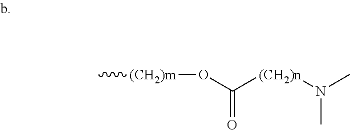
type 3 ionizable head; 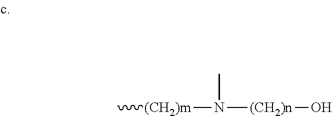
type 4 ionizable head,
if W¹ and Y are not bonded to each other, then:
W¹ is H;
W² is O or S or NH or NR², wherein R² is a C₁ to C₄ small alkyl optionally substituted with an OH group;
Group 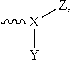
wherein the wavy line represents the bond to W², is a group chosen from among structures d-i below, wherein the wavy line represents the bond to W²: 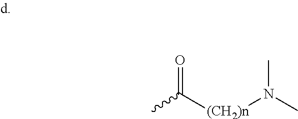
if W² is O, type 1 ionizable head; 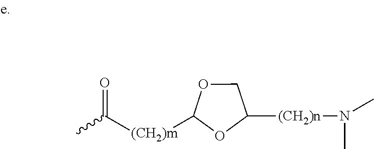
if W² is O, type 5 ionizable head; 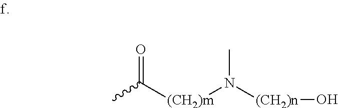
if W² is O, type 6 ionizable head; 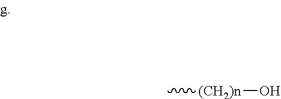
if W² is NH or NR², type 7 ionizable head; 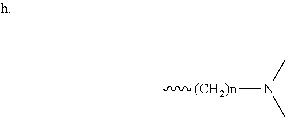
if W² is NH or NR², type 8 ionizable head; 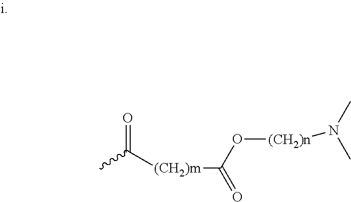
if W² is O, type 9 ionizable head;
if A³ is N, then:
R⁵ is H or (CH₂)_{q}—OH, wherein index q ranges from q=2 to q=8
W¹ and Y are absent,
W², X and Z together form a group of structure (CR^{a}R^{b})ₜ-A³ with index t varying from t=1 to t=6, R^{a} and R^{b} independently equal to H or small C₁-C₅ alkyl or cycloalkyl, and with Z equal to H, or OH, or NR′R″, wherein R′ and R″ are small C₁-C₅ alkyls or cycloalkyls, or wherein R′ and R″ are branches of a heterocyclic group that incorporates the N atom to which R′ and R″ are bound, such as pyrrolidine, piperidine, morpholine, and the like.

2. The lipid of claim 1, wherein M of Formula A or Formula D incorporates 1 to 3 heteroatoms selected from N, O and S.

3. The lipid of claim 2, wherein the heteroatoms are part of a functional group that is an ester, ether, amide, amine or thioether.

4. The lipid of claim 1, wherein the heteroatoms are part of a functional group that are selected from OH, O-alkyl and S-alkyl substituents.

5. The lipid of claim 1, wherein M of Formula A or Formula D are independently selected from a moiety of Formula C: 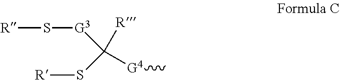
wherein:
R′ and R″ are, independently, a linear or branched optionally substituted C₃ to C₂₀ or C₃ to C₁₂ alkyl group, optionally incorporating from 1 to 3 C═C double bonds of E or Z geometry;
R′″ is H or a linear, branched, or cyclic optionally substituted C₁ to C₆ alkyl group;
G³ and G⁴ are, independently, a group of structure (CR^{c}R^{f})ₛ with R^{c} and R^{f} are each independently selected from H or optionally substituted C₁-C₅ alkyl or cycloalkyl, and wherein s is 0 to 6.

6. The lipid of claim 5, wherein R′″ is substituted with heteroatoms selected from N, O and/or S.

7. The lipid of claim 1, wherein A² is N, W¹ and Y are absent, W² and X together form a group of the structure (CR^{c}R^{d})ᵣ, and Z is NR′R″ and wherein the heterocyclic group that incorporates the N atom to which R′ and R″ are bound is pyrrolidine, piperidine or morpholine.

8. The lipid of claim 1 having a structure of any one of compounds 5-44 below or pharmaceutically acceptable salts thereof:

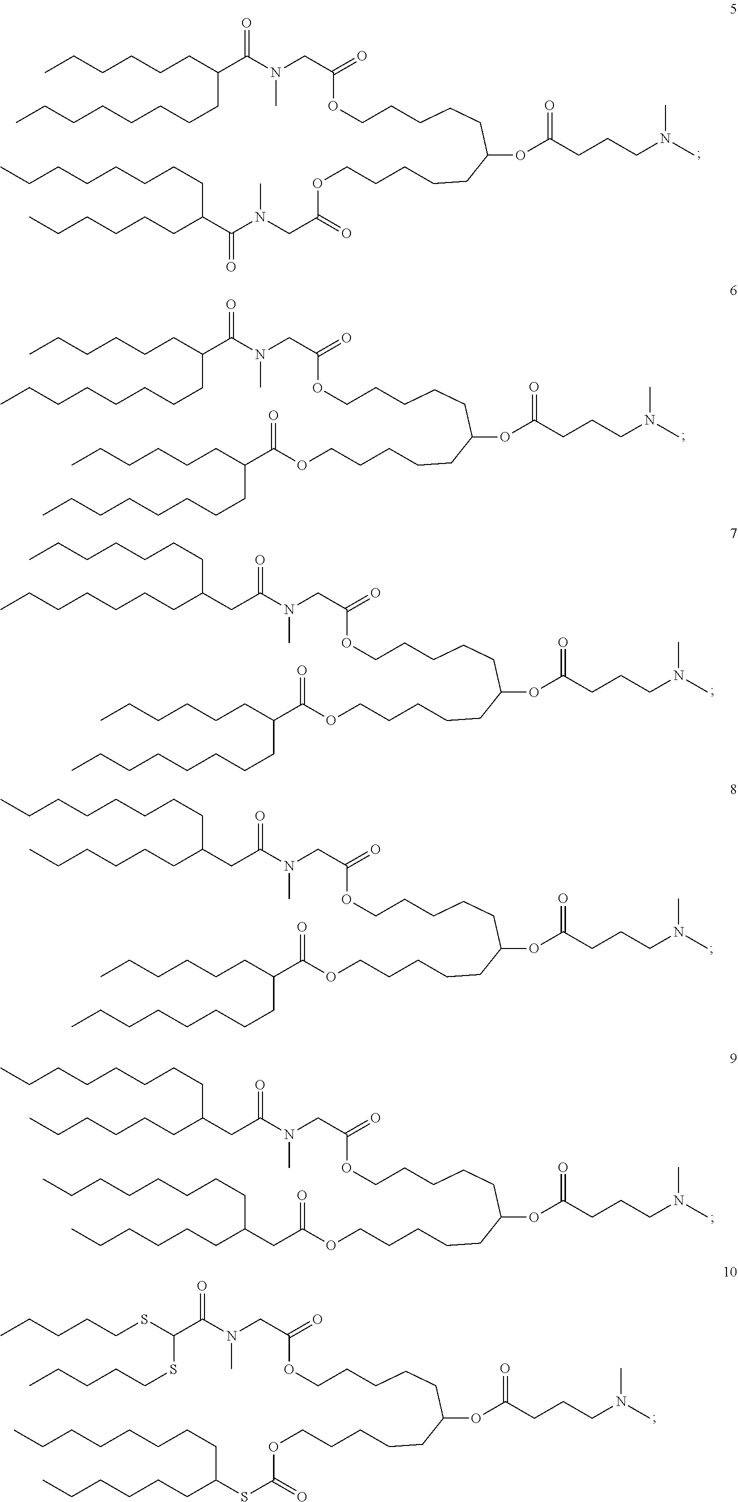

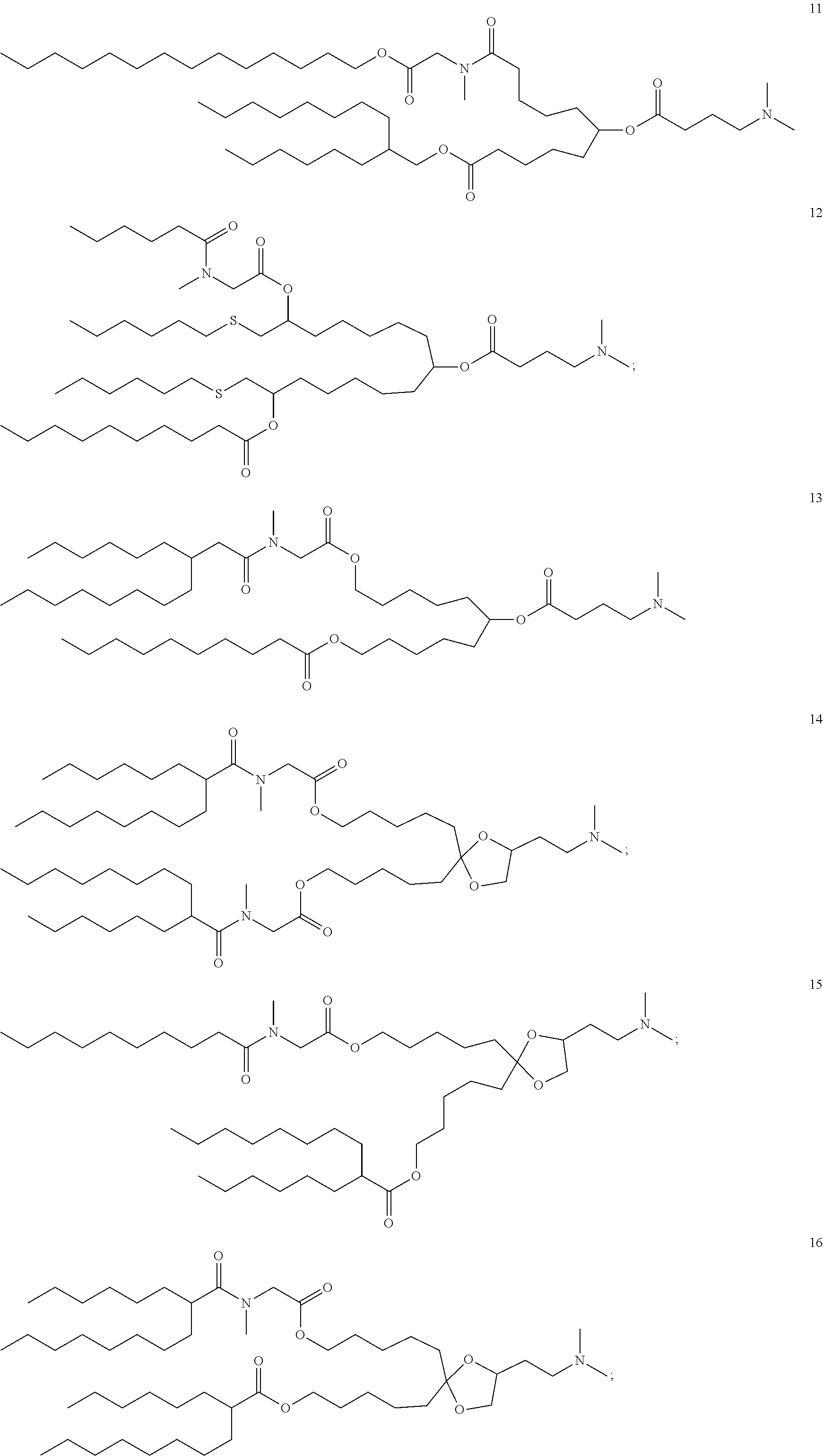

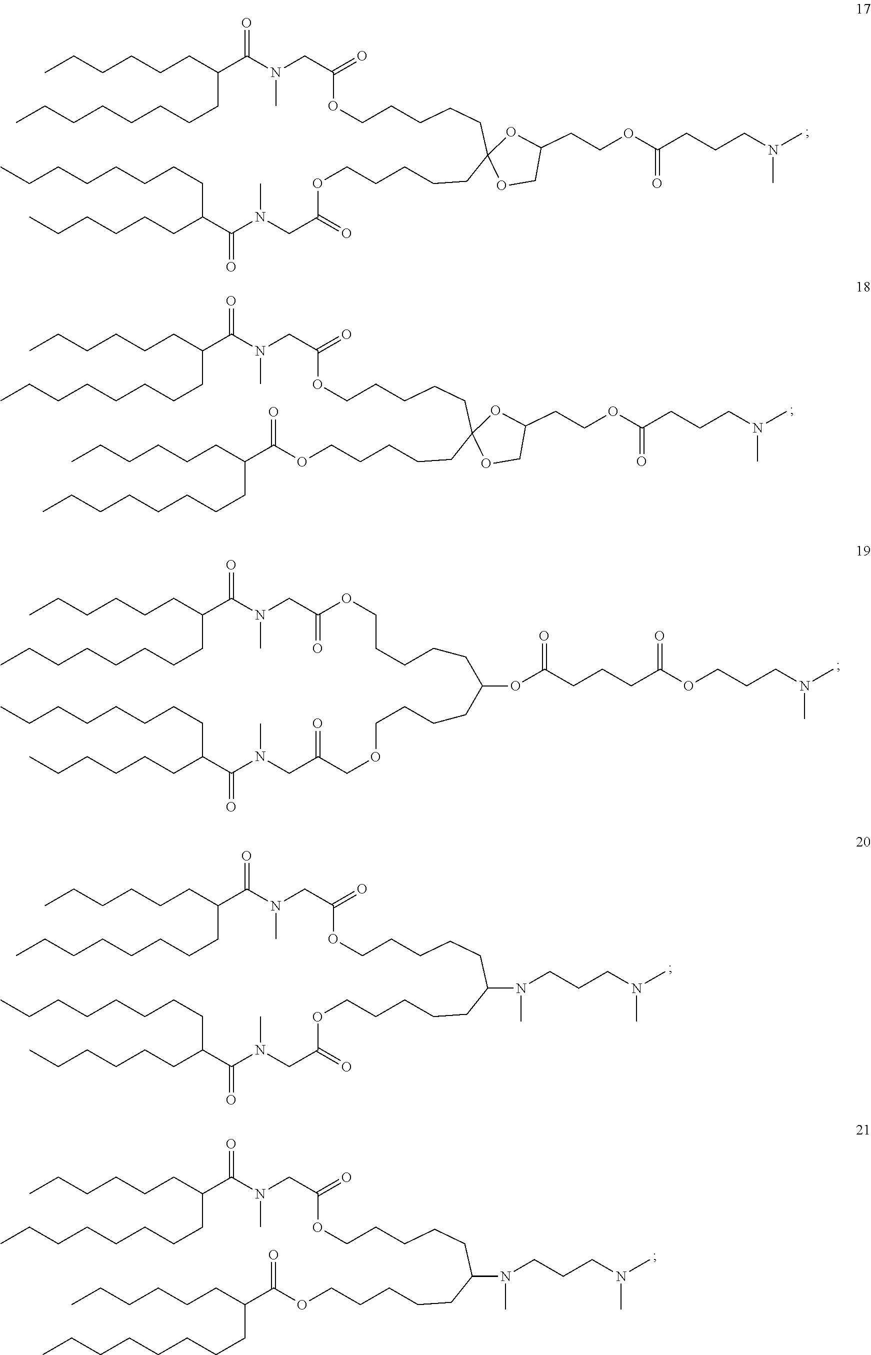

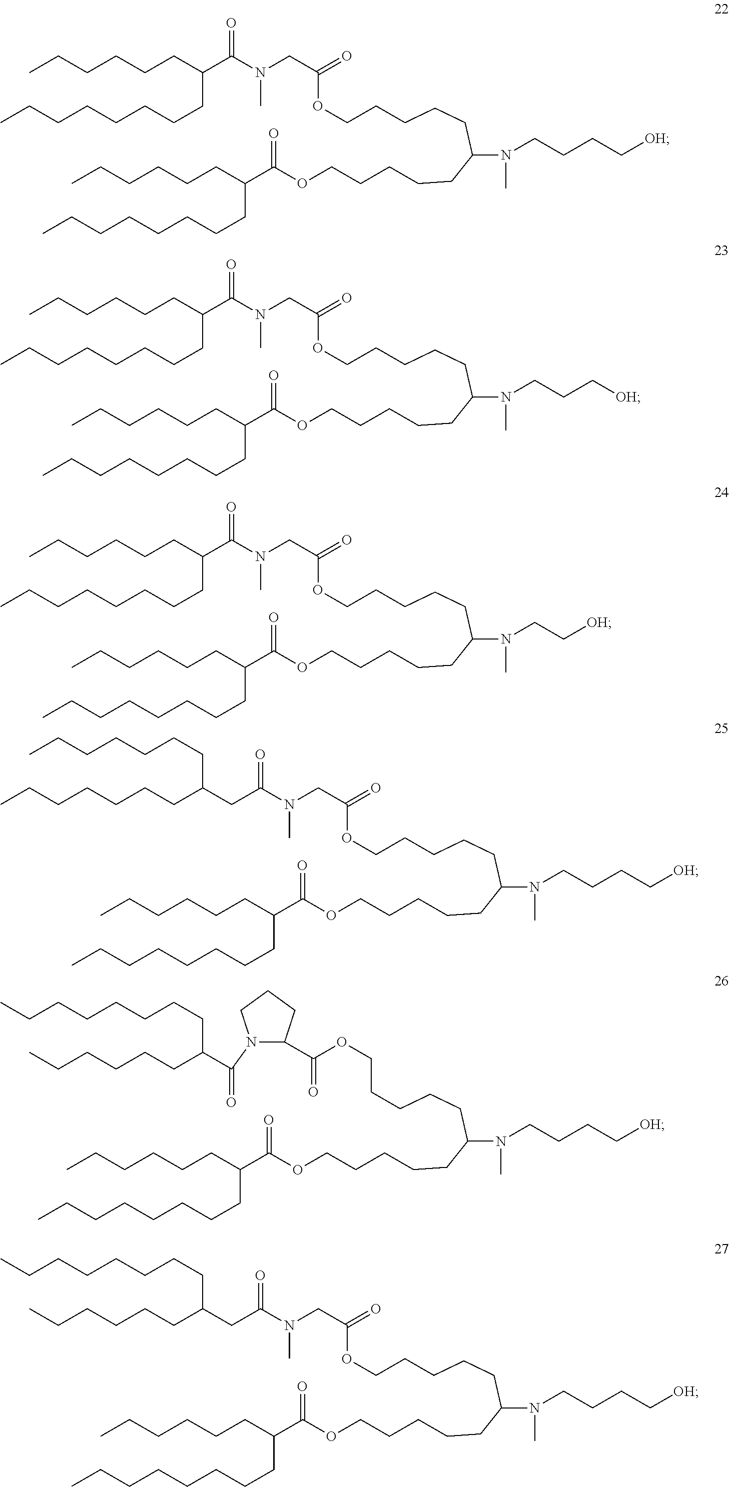

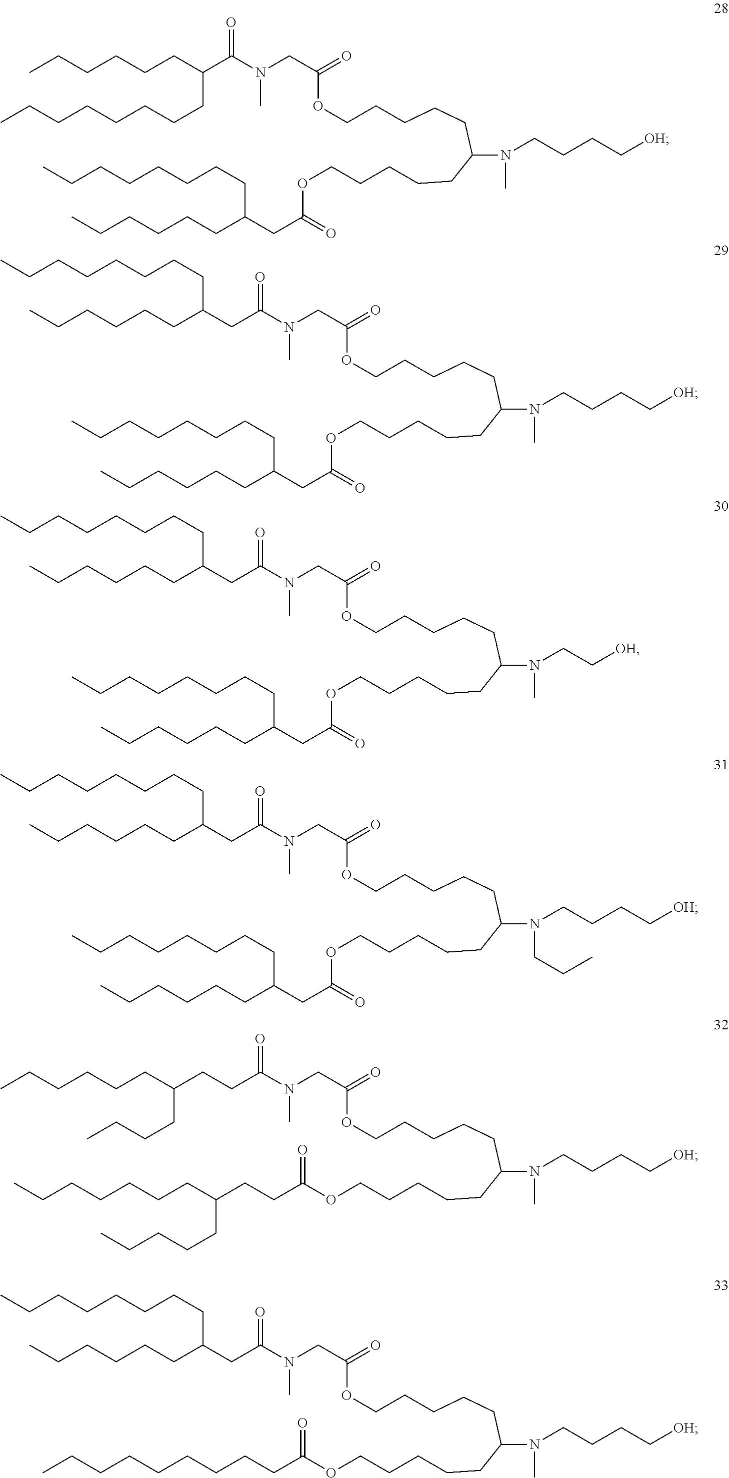

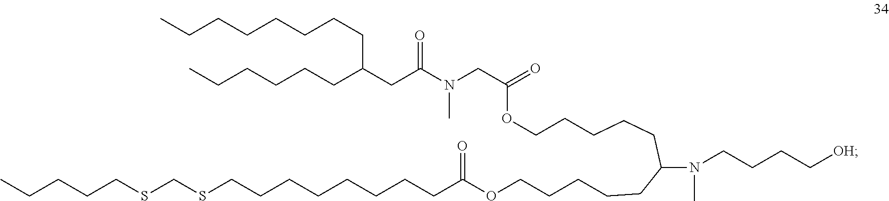 [SMILES: CCCCCCCCC(CCCCCC)CC(=O)N(C)CC(=O)OCCCCCC(CCCCCOC(=O)CCCCCCCCSCSCCCCC)N(C)CCCCO]

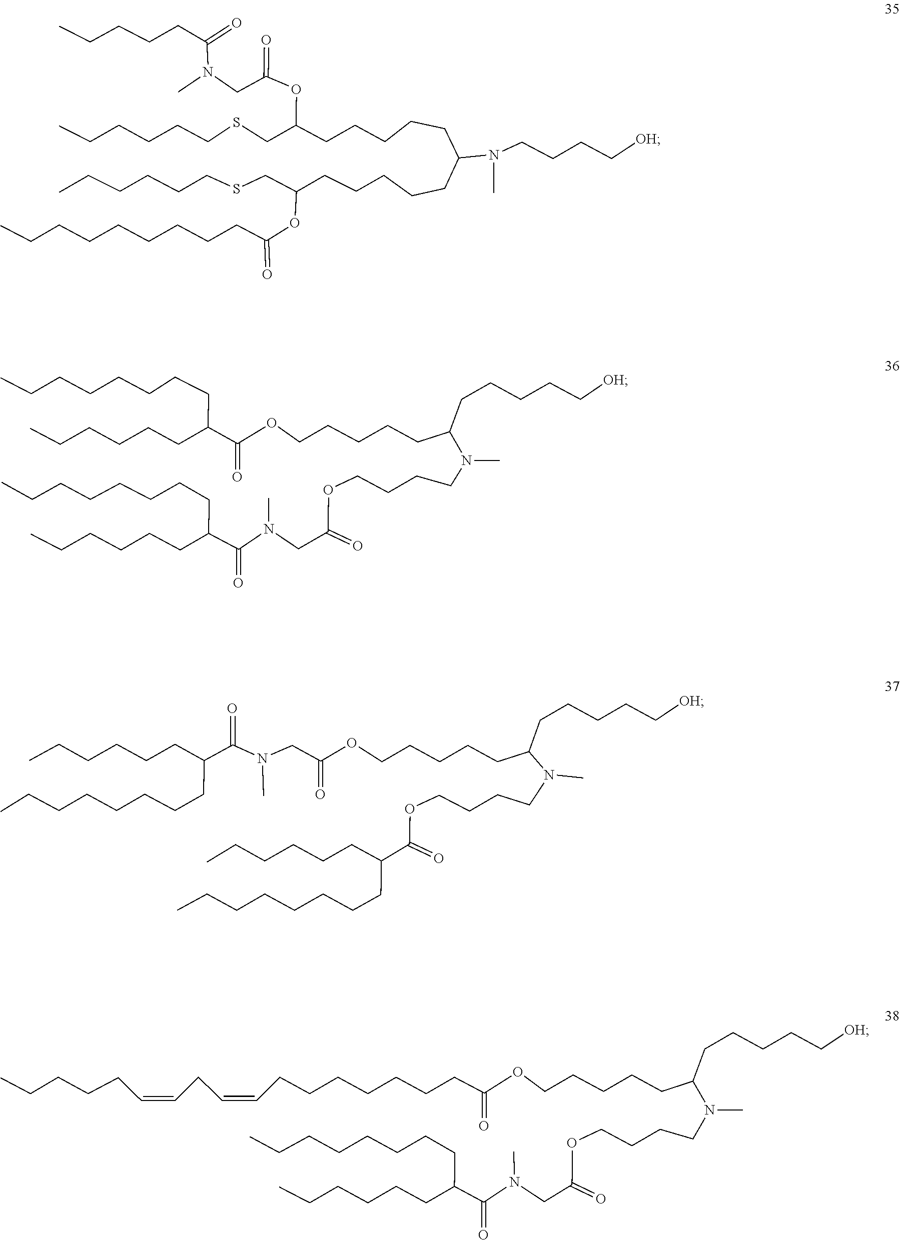 [SMILES: CCCCCCCCCC(=O)OC(CCCCCC(CCCCCC(CSCCCCCC)OC(=O)CN(C)C(=O)CCCCC)N(C)CCCCO)CSCCCCCC ; CCCCCCCCC(CCCCCC)C(=O)OCCCCCC(CCCCCO)N(C)CCCCOC(=O)CN(C)C(=O)C(CCCCCC)CCCCCCCC ; CCCCCCCCC(CCCCCC)C(=O)OCCCCN(C)C(CCCCCO)CCCCCOC(=O)CN(C)C(=O)C(CCCCCC)CCCCCCCC ; CCCCC/C=C\C/C=C\CCCCCCCC(=O)OCCCCCC(CCCCCO)N(C)CCCCOC(=O)CN(C)C(=O)C(CCCCCC)CCCCCCCC]

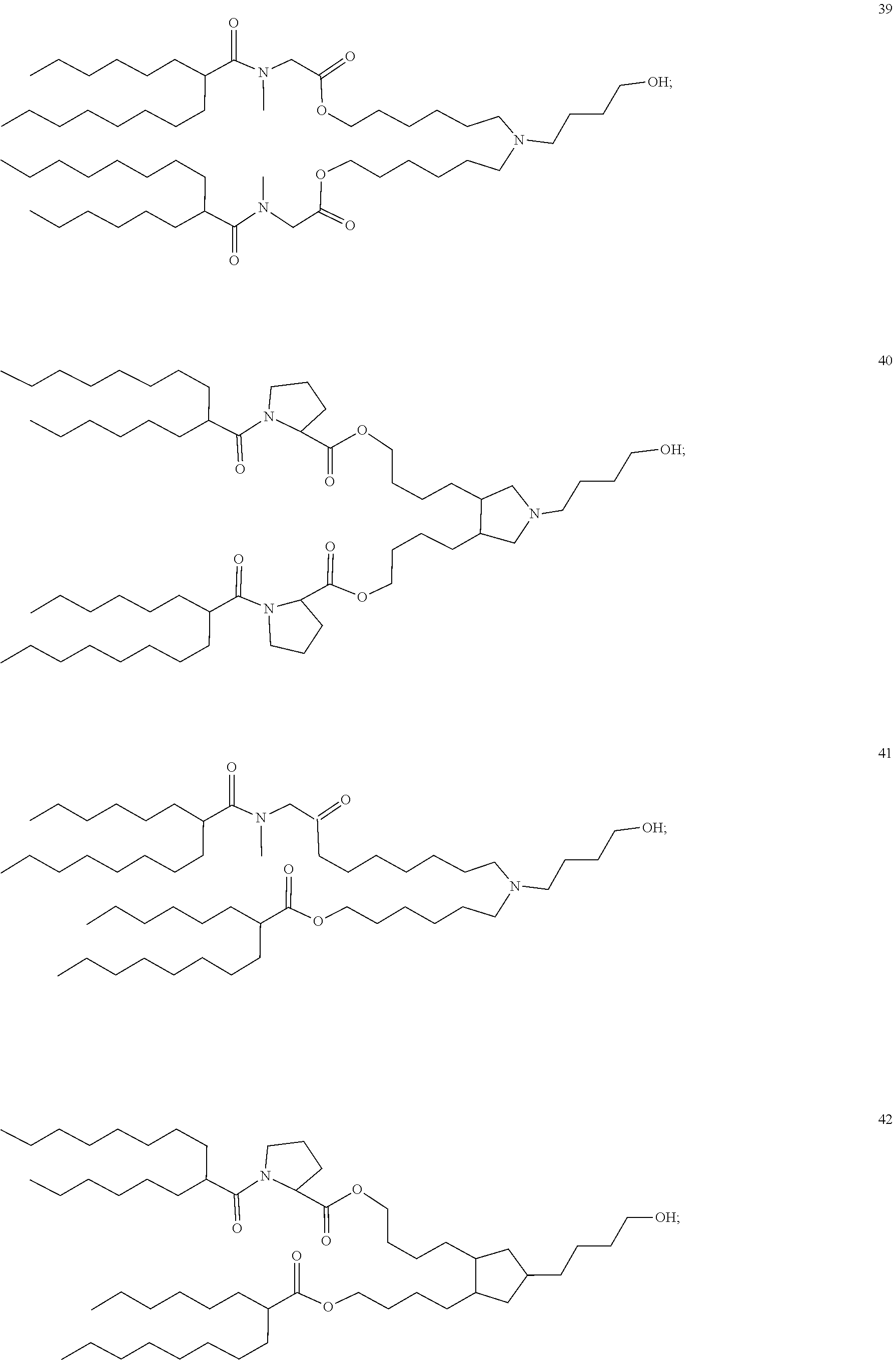

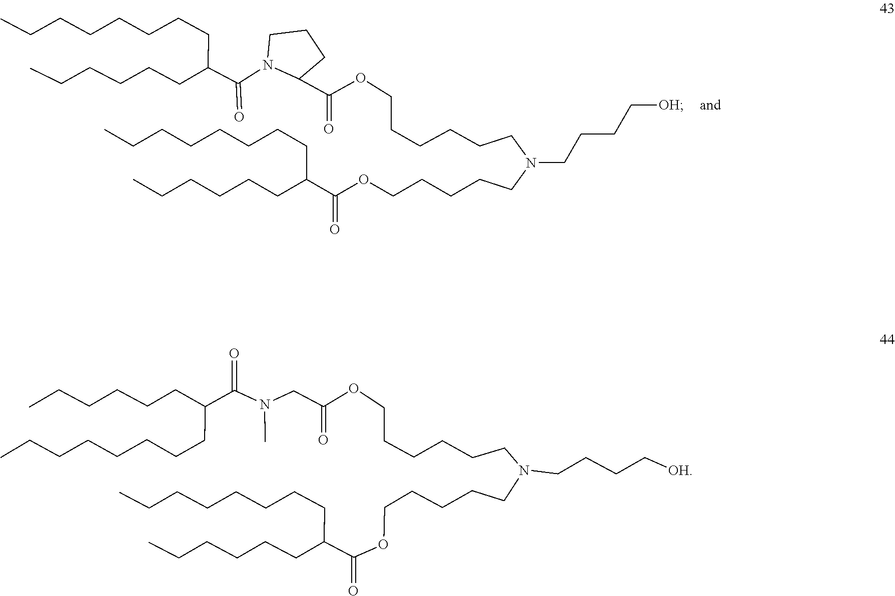 [SMILES: CCCCCCCCC(CCCCCC)C(=O)OCCCCCN(CCCCO)CCCCCCOC(=O)C1CCCN1C(=O)C(CCCCCC)CCCCCCCC ; "and" ; CCCCCCCCC(CCCCCC)C(=O)OCCCCCN(CCCCO)CCCCCCOC(=O)CN(C)C(=O)C(CCCCCC)CCCCCCCC]

9. A lipid or a pharmaceutically acceptable salt thereof comprising:
a protonatable amino head group;
two lipophilic chains, wherein the amino head group has a central nitrogen atom or carbon atom to which each of the two lipophilic chains are directly bound;
at least one of the lipophilic chains has the formula: 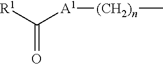
n is 4 to 8;
A¹ is a bond or O;
if A¹ is O, then
R¹ is an amino acid-derived moiety as depicted in Formula B, 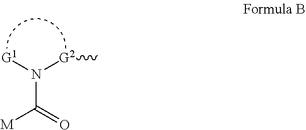
wherein the wavy line depicts a bond between the C═O group of the lipophilic chain and G² of Formula B,
the dashed semicircle between G¹ and G² is present or absent,
wherein when the dashed semicircle is absent:
G¹ is a C₁-C₆ alkyl or cycloalkyl, optionally substituted with one or more heteroatoms selected from N, O and S;
G² is (CR^{a}R^{b})ₚ, wherein R^{a} and R^{b} are, independently, H or optionally substituted C₁-C₆ alkyl or cycloalkyl, and p is 1 to 5;
wherein when the dashed semicircle is present:
an optionally substituted ring structure is present that includes the N atom, G¹ and G²;
M is a linear or branched optionally substituted C₅ to C₂₀ alkyl group, optionally incorporating from 1 to 3 C═C double bonds of E or Z geometry;
if A¹ is a bond, then:
R¹ is an amino acid-derived moiety as depicted in Formula D, 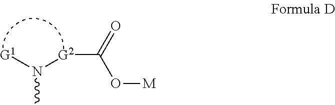
wherein the wavy line denotes a bond between the N atom of Formula D and the C═O group of the lipophilic chain;
wherein G¹, G² and M are defined above in relation to Formula B; and
the dashed semicircle between G¹ and G² denotes that a first atom that is part of G¹ is optionally bonded to a second atom that is part of G², so as to form a ring structure that includes the N atom;
each lipophilic chain has between 12 and 30 carbon atoms in total; and
wherein the lipid has (i) a pKₐ of between 6 and 8; and (ii) a log P of at least 11.

10. The lipid of any one of claim 1, wherein the lipid, when formulated in a lipid nanoparticle comprising an mRNA, results in an increase in biodistribution of the lipid nanoparticle of at least about 10% in the liver and/or one or more extrahepatic tissues relative to an otherwise identical lipid nanoparticle containing DLin-MC3-DMA, ALC-0315 or SM-102 as measured by luminescence of the mRNA in vivo in the liver and/or the one or more extrahepatic tissues.

11. A lipid nanoparticle comprising the lipid of claim 1 and a nucleic acid.

12. The lipid nanoparticle of claim 11, comprising a helper lipid and optionally a hydrophilic polymer-lipid conjugate.

13. The lipid nanoparticle of claim 12, wherein the helper lipid is selected from cholesterol, a diacylglycerol, a glycerophospholipid-cholesterol conjugate and a sphingolipid.

14. A lipid nanoparticle comprising:
an ionizable lipid with two lipophilic chains, at least one of the chains comprising an amino acid-derived moiety selected from a sarcosine group and a proline group or derivatives thereof in one or both of the lipophilic chains;
one or more helper lipids;
optionally a hydrophilic polymer-lipid conjugate; and
a nucleic acid.

15. A method for administering a therapeutic or prophylactic nucleic acid to a subject in need thereof, the method comprising preparing or providing the lipid nanoparticle of claim 11 comprising the nucleic acid and administering the lipid nanoparticle to the subject.

16. A method for delivering a cargo molecule to a cell, the method comprising contacting the lipid nanoparticle of claim 11 with the cell in vivo or in vitro.

17. The method of claim 16, wherein the cargo molecule is a nucleic acid.

18. (canceled)

19. (canceled)

20. (canceled)
